(19) United States
(12) Patent Application Publication
(10) Pub. No.: US 2024/0026029 A1
(43) Pub. Date: Jan. 25, 2024

(54) MEANS AND METHODS FOR TREATING SUBJECTS WITH ERBB3 MUTATION POSITIVE CANCER

(71) Applicant: Merus N.V., Utrecht (NL)

(72) Inventor: Ernesto Isaac WASSERMAN, Utrecht (NL)

(21) Appl. No.: 18/251,832

(22) PCT Filed: Nov. 3, 2021
(86) PCT No.: PCT/NL2021/050674

§ 371 Date: May 4, 2023

(30) Foreign Application Priority Data
Nov. 4, 2020 (NL) 2026824

(51) Int. Cl. C07K 16/32; A61P 35/00

(52) U.S. Cl. CPC C07K 16/32; A61P 35/00; C07K 2317/31

(57) ABSTRACT

The invention relates to the field of therapeutic (human) antibodies for the treatment of a subject with an ERBB3 mutation positive cancer. More in particular it relates to treating cancers comprising an ERBB3 mutation that drives oncogenesis. The antibodies are bispecific antibodies that comprises an antigen binding site that can bind an extracellular part of ERBB2 and an antigen binding site that can bind an extracellular part of ERBB3.

### CROSS REFERENCE TO RELATED APPLICATIONS

This application is the national stage of International Application No. PCT/NL2021/050674, filed Nov. 3, 2021, which claims priority to NL Application No. 2026824, filed Nov. 4, 2020.

### REFERENCE TO SEQUENCE LISTING SUBMITTED ELECTRONICALLY

The content of the electronically submitted sequence listing (Name: “4096_0490001_SequenceListing_ST25.txt”; Size: 136,345 bytes; and Date of Creation: Apr. 28, 2023) is herein incorporated by reference in its entirety.

### BACKGROUND OF THE INVENTION

The invention relates to the field of antibodies. In particular it relates to the field of therapeutic (human) antibodies for the treatment of diseases involving aberrant cells. More in particular it relates to antibodies that bind ERBB2 and ERBB3 and their use in the treatment of cancer.

ERBB3 is a transmembrane receptor tyrosine kinase of the Erb family, which also includes Epidermal growth factor receptor (EGFR), ERBB2 and ERBB4. Similar to the other proteins of the family, ERBB3 contains an extracellular domain, transmembrane and intracellular tyrosine kinase domain. Upon interaction with its ligand Neuregulin (NRG), ERBB3 heterodimerizes with other Erb family members activating the intracellular tyrosine kinase domains, which undergo autophosphorylation, and in turn activate downstream signaling pathways including mitogen-activated protein kinase (MAPK) and the PI3K pathway. Upregulation of the ErbB pathway results in oncogenic proliferative signaling and occurs frequently in cancer cells.

ERBB3 gene mutations are understood to be oncogenic drivers with varying levels of prevalence found in a wide range of tumors. Several of these mutations are present in the ligand binding extracellular domain of ERBB3 but mutations are also observed in the intracellular tyrosine kinase domain. Interestingly, it is postulated that ERBB3 lacks an intrinsic tyrosine kinase activity. Nevertheless, mechanistically, these mutations in ERBB3 are believed to result in constitutive activation of the ERBB2 receptor signaling and oncogenesis either by ligand-independent heterodimerization with ERBB2 or induction of ERBB2 kinase domain. Other as of yet unknown functional mechanisms of action of ERBB3 mutations have not been ruled out.

Therapeutic importance of ERBB3 mutations has been explored using tyrosine kinase inhibitors (TKI), which may inhibit several ErbB family receptors. These include, but are not limited to afatinib, neratinib, lapatinib, erlotinib, gefitinib or osimertinib. In one study by Choudhury, Noura J et al. (Journal of clinical oncology, vol. 34, 18 (2016): 2165-71), treatment with afatinib, of patients with extracellular ERBB3 mutations, resulted in a clinical response. However, all patients in this study developed resistance after afatinib treatment. In another basket study, no clinical response was observed after treatment with Neratinib (a pan-ErbB family inhibitor) in patients harboring ERBB3 mutations (Hyman, David M et al. Nature, vol. 554, 7691 (2018): 189-194). The above studies indicate that response to TKI inhibitors is mixed and inconclusive in patients harboring ERBB3 mutations.

A few studies have also explored the therapeutic potential of monoclonal antibodies in ERBB3 mutation harboring patients generally showing limited efficacy. However, in a large retrospective study (Verlingue, Loic et al. European journal of cancer, vol. 92 (2018): 1-10) patients harboring mutations in ERBB3 extracellular domain received monoclonal antibody treatment with trastuzumab and/or lapatinib or afatinib and although a significant response to treatment was observed in certain patients which harbored mutations in the tyrosine kinase domain of ERBB3, patients with mutations in the extracellular domain, failed to respond to treatment.

Tyrosine kinase inhibition using pan-TKI inhibitors, such as afatinib, erlotinib, gefitinib, lapatinib, osimertinib, and neratinib, in ERBB3 mutated patients have also demonstrated limited efficacy. Further, it is also well known that treatment in patients with TKI inhibitors results in toxicity and resistance to therapy leading to recurrence or metastasis. Hence there is a need for more robust targeted treatments based on ERBB3 driver mutations with a potential to inhibit ErbB signaling axis. Currently however, there is no specific ErbB mutation directed therapy authorized anywhere in the world, and many patients with cancer harboring ErbB mutations are managed with non-specific cancer therapy.

Accordingly, there is a need in the field for treatment of cancers harboring ERBB3 mutations.

### SUMMARY OF THE INVENTION

In the present disclosure, the inventors have identified that use of an anti-ERBB2 anti-ERBB3 targeting molecule successfully treats cancer cells that are ERBB2 and ERBB3 positive and harbor an ERBB3 mutation, preferably where the targeting molecule targets domain 1 of ERBB2 and domain 3 of ERBB3 thereby sterically preventing heterodimerization, which can be promoted by an ERBB3 mutation and/or occur in a ligand independent fashion.

The present disclosure provides a method for the treatment of a subject that has an ERBB2 and ERBB3 positive cell, the method comprising administering a bispecific antibody that comprises a first antigen-binding site that can bind an extracellular part of ERBB2, preferably at domain 1, and a second antigen-binding site that can bind an extracellular part of ERBB3, preferably at domain 3, the method characterized in the cell that comprises an ERBB3 mutation. Typically the mutation comprises the extracellular domain of the ERBB3 protein, or is one that is intracellular, which promotes and/or is correlated with ligand independent heterodimerization of ERBB2 and ERBB3, such that it promotes the PI3K pathway. Preferably, the ERBB3 mutation positive cell, lacks other oncogenic driver mutations and/or amplifications.

Preferably, a mutation in ERBB3 comprises one or more mutations compared to the non-mutated sequence according to SEQ ID No: 1. Thus, a mutation in ERBB3 is a mutation over said non-mutated sequence.

The subject is preferably a human subject.

The ERBB3 mutation of the present disclosure is preferably an ERBB3 driver mutation, that comprises a mutation which promotes and/or is correlated with ligand independent heterodimerization of ERBB2 and ERBB3 and/or activation of the ERBB2 kinase domain, such that it promotes the activation of the ERBB signaling axis and downstream mitogen-activated protein kinase (MAPK) and the PI3K pathway signaling.

The ERBB3 mutation preferably occurs in the extracellular domain of ERBB3, preferably in domains DI-DIV, where domain I comprises residues 56-166, domain II comprises residues 182-332, domain III comprises residues 353-472 and domain IV comprises residues 499-629 (FIG. 5). More preferably, the mutation is not at a residue critical for the binding of the therapeutic molecule, such that it may diminish affinity and activity of the therapeutic compound.

More preferably the mutation occurs at positions involved in active formation of ERBB3 receptor-ligand complex and/or homo/heterodimerization of ERBB3, comprising positions M60, M91, R103, A232, P262, G284, and D297.

In another aspect of the invention, the ERBB3 mutation comprises a mutation in the intracellular domain of ERBB3 comprising amino acid residues 710-964, preferably at positions Q809, S846, Q865 or E928.

In some aspect, the ERBB3 mutation comprises a mutation that is oncogenic driver, comprising amino acid residues M60, M91, R103, V104, R135, F219, H228, A232, P262, G284, D297, K329, E332, T355, R475, Q809, S846, Q865 or E928. More preferably the ERBB3 mutations are hotspot mutations comprising V104, A232, G284, D297, K329, T355, S846 or E928 (FIG. 5).

Specifically, ERBB3 mutation comprises any one of the following:
M60N, where N is any naturally occurring amino acid, preferably K;
M91N, where N is any naturally occurring amino acid, preferably I;
R103N, where N is any naturally occurring amino acid, preferably G;
V104N, where N is any naturally occurring amino acid, preferably L or M;
R135N, where N is any naturally occurring amino acid, preferably C;
F219N, where N is any naturally occurring amino acid, preferably L;
H228N, where N is any naturally occurring amino acid, preferably Q;
A232N, where N is any naturally occurring amino acid, preferably V;
P262N, where N is any naturally occurring amino acid, preferably H or L or S;
G284N, where N is any naturally occurring amino acid, preferably R;
D297N, where N is any naturally occurring amino acid, preferably Y or A or H or N or V;
K329N, where N is any naturally occurring amino acid, preferably E or I or T;
E332N, where N is any naturally occurring amino acid, preferably K;
T355N, where N is any naturally occurring amino acid, preferably A or I or P;
R475N, where N is any naturally occurring amino acid, preferably W;
Q809N, where N is any naturally occurring amino acid, preferably R;
S846N, where N is any naturally occurring amino acid, preferably I;
Q865N, where N is any naturally occurring amino acid, preferably H;
E928N, where N is any naturally occurring amino acid, preferably G.

Preferably, the mutation in ERRB3 is selected from a mutation in the extracellular domain of ERBB3, more preferably a mutation in domain I, domain II, domain III or domain IV.

Preferably, the mutation in ERRB3 is selected from a mutation in domain I (DI), more preferably a mutation at position M60, M91, R103, V104, R135.

Preferably, the mutation in ERRB3 is selected from a mutation in domain II (DII), more preferably a mutation at position F219, H228, A232, P262, G284, D297, K329 and E332.

Preferably, the mutation in ERRB3 is selected from a mutation in domain III (DID), more preferably a mutation at position T355.

Preferably, the mutation in ERRB3 is selected from a mutation in domain IV (DIV), more preferably mutation at position R475.

Preferably the mutation in ERRB3 is selected from a mutation in the intracellular domain of ERBB3, more preferably a mutation in the tyrosine kinase domain.

Preferably, the ERBB3 mutation comprises a mutation in the tyrosine kinase domain of ERBB3 comprising amino acid residues 710-964, preferably at positions Q809, 5846, Q865 or E928.

Preferably, the mutation in the intracellular domain is selected from a mutation selected from Q809, S846, Q865 and E928.

In an aspect of the disclosure, ERBB3 mutations at position R426 are excluded.

Preferably cancers harboring ERBB3 mutations lack another oncogenic driver, including, but not limited to KRAS, NRAS, PIK3CA, and/or BRAF or the cell shows absence of oncogenic mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, and PTEN.

Preferably cancers harboring ERBB3 mutations lack the following oncogenic amplifications c-MET amplification, c-MYC amplification, EGFR amplification, ERBB2 amplification, and/or MDM2 amplification.

Preferably cancers harboring ERBB3 mutations do not have PTEN loss.

The bispecific antibody that comprises a first antigen-binding site that binds an extracellular part of ERBB2 and a second antigen-binding site that binds an extracellular part of ERBB3 preferably has a first antigen-binding site that binds domain I of ERBB2 and a second antigen-binding site that binds domain III of ERBB3. In some embodiments the affinity of the first antigen-binding site for ERBB2 is lower than the affinity of the second antigen-binding site for ERBB3.

The bispecific antibody preferably comprises
i) at least the CDR1, CDR2 and CDR3 sequences of an ERBB2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898 or wherein said antibody comprises CDR sequences that differ in at most 3 amino acids, preferably in at most 2 amino acids, preferably in at most 1 amino acid from the CDR1, CDR2 and CDR3 sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) at least the CDR1, CDR2 and CDR3 sequences of an ERBB3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

In one aspect, the bispecific antibody comprises
i) an ERBB2 specific heavy chain variable region sequence selected from the group consisting of the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 and MF1898, or wherein said antibody comprises a heavy chain variable region sequence that differs in at most 15 amino acids from the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) an ERBB3 specific heavy chain variable region sequence selected from the group consisting of the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

A preferred bispecific antibody for use in the present invention is MF3958×MF3178, which comprises heavy chain variable regions MF3958 (anti-ERBB2) and MF3178 (anti-ERBB3). MF3958×MF3178 has been demonstrated to be well tolerated as a single agent, with low risk for immunogenicity in the treatment of over 100 patients, making it an excellent agent for combination therapy, providing an advantage over other anti-ERBB2 and/or anti-ERBB3 targeting agents. Without being bound to any theory, MF3958×MF3178's efficacy in the treatment of patients having cancer harboring an ERBB2 and ERBB3 positive cell having an ERBB3 mutation is thought to be based on the epitope specificity of MF3958×MF3178 and imbalance of affinity, permitting MF3958×MF3178 to dock onto domain 1 of ERBB2, and block ERBB3 at domain 3 from dimerizing with ERBB2 thereby disrupting activation of the PI3K pathway.

The variable domain that comprises said first antigen binding site and the variable domain that comprises said second antigen binding site of said bispecific antibody preferably comprise a light chain variable region comprising a CDR1 having the sequence (RASQSISSYLN), a CDR2 having the sequence (AASSLQS), and a CDR3 having the sequence (QQSYSTPPT), according to KABAT numbering or according to the IMGT numbering system, the CDRs are QSISSY, AAS and QQSYSTPPT, respectively.

The variable domain that comprises said first antigen binding site and the variable domain that comprises said second antigen binding site of said bispecific antibody preferably comprise a light chain variable region of FIG. 6a or FIG. 6b.

The invention further provides a method for the treatment of a subject that has an ERBB2 and ERBB3 positive cell, preferably a cancer cell, the cell comprising an ERBB3 mutation, with a bispecific antibody that comprises a first antigen-binding site that binds an extracellular part of ERBB2, and a second antigen-binding site that binds an extracellular part of ERBB3, as a first line therapy. The invention further provides a method of treatment of a subject having a cancer harboring a ERBB3 mutation, the method comprising administering a bispecific antibody that comprises an antigen binding site that can bind an extracellular part of ERBB2 and an antigen binding site that can bind an extracellular part of ERBB3, wherein said treatment is provided as a first line therapy. The method includes treatment with a bispecific antibody that comprises a first antigen-binding site that binds an extracellular part of ERBB2 and a second antigen-binding site wherein the patient's ERBB3 mutation harboring cancer has progressed after having received a prior treatment, preferably comprising chemotherapy, checkpoint inhibitor therapy (including anti-PD1 and anti-PD-L1 approved therapies and applicable therapies in clinical development), anti-ERBB2 or anti-ERBB3 or anti-VEGFR2 (vascular endothelial growth factor receptor 2), or a prior treatment with a tyrosine kinase inhibitor (TKI) of ERBB2 or of VEGFR2-TIE2, or with a combination thereof.

The chemotherapy according to the present invention preferably comprises gemcitabine, capecitabine, carboplatin, a taxane, such as docetaxel or paclitaxel, 5-fluorouracil (with or without radiotherapy), vinorelbine, carmustine, doxorubicin, epirubicin, mitoxantrone, vinblastine, cisplatin (or pemetrexed), oxaliplatin, carboplatin, ifosfamide, mytomycin C, vindesine, etoposide, Folfox (i.e. a combination of 5-fluorouracil, leucovorin, and oxaliplatin) or Folfiri (i.e. a combination of leucovorin, 5-fluorouracil and irinotecan), Folfirinox (a combination of leucovorin, 5-fluorouracil, irinotecan and oxaliplatin) or any combination thereof.

The checkpoint inhibitor therapy according to the present invention includes an anti-PD1, anti-PD-L1, and anti-CTLA4 therapeutic moiety and preferably comprises ipilimumab, nivolumab, pembrolizumab, atezolizumab, avelumab, durvalumab or cemiplimab, or any combination thereof.

ERBB2 targeted treatment is preferably with a monospecific bivalent antibody comprising antigen-binding sites that bind an extracellular part of ERBB2 is preferably trastuzumab, pertuzumab, or trastuzumab-emtansine. The ERBB2 TKI is preferably one or more of lapatinib, canertinib, neratinib, tucatinib (irbinitinib), CP-724714, tarloxitinib, mubritinib, afatinib, varlitinib, and dacomitinib, preferably afatinib. In one aspect the ERBB2 TKI is afatinib. An ERBB2 TKI may also affect ERBB1 signaling but is different from an ERBB1 TKI in that it has significant activity on ERBB2.

The ERBB3 targeted treatment is preferably with a monospecific bivalent antibody comprising antigen-binding sites that bind an extracellular part of ERBB3 preferably comprises patritumab, seribantumab, lumretuzumab, elgemtumab, GSK2849330, KTN3379 or AV-203.

The VEGFR2 targeted treatment is preferably with a monospecific bivalent antibody comprising antigen-binding sites that bind an extracellular part of VEGFR2, preferably comprises ramucirumab. The VEGFR2-TIE2 TKI is preferably regorafenib.

A target specific treatment may be combined with other treatments for the same target. Alternatively a treatment that targets one target may also be active on another of the mentioned targets.

The cancer is preferably a recurrent cancer or a metastasized cancer or a combination of the two. Recurrence typically refers to local recurrence and means that the cancer is in the same place as the original cancer or very close to it. A tumor is typically said to be a metastasized tumor when the tumor has migrated to lymph nodes or tissues near the original cancer or spread to more distant organs or tissues far from the original cancer.

The cancer harboring an ERBB3 mutation includes colorectal cancer, gastric, non-small-cell lung (NSCLC) adenocarcinoma (adeno), NSCLC (squamous), renal carcinoma, melanoma, ovarian, lung large cell, esophageal, small-cell lung cancer, hepatocellular (HCC), breast cancer, hormone-positive breast cancer, glioblastoma, and head and neck cancer.

The cancer harboring an ERBB3 mutation is preferably a gastric cancer such as a gastric adenocarcinoma or an esophago-gastric cancer, a pancreatic cancer, a pancreatic ductal adenocarcinoma, a sarcoma, a bladder cancer, a colorectal cancer, a gallbladder cancer, head and neck cancer, a prostate cancer, uterine/endometrial cancer, a breast cancer, an ovarian cancer, a liver cancer, a lung cancer such as non-small cell lung cancer, preferably a non-small cell lung cancer, including invasive mucinous adenocarcinoma.

### BRIEF DESCRIPTION OF THE DRAWINGS

FIG. 1 sets out prevalence of ERBB3 mutations in solid tumors comprising ERBB2, ERBB3 positive cells.

FIG. 2. sets out the inactive and activated form of ERBB3 and its heterodimerization with ERBB2 and sets out the mechanism of action whereby therapeutic molecules of the inventive method disclosed binds domain I of ERBB2 and domain III of the inactive form of ERBB3 thereby preventing heterodimerization, including in the presence of ERBB3 mutations in the extracellular and/or intracellular domain that promote heterodimerization in the presence of ligand or in a ligand independent manner.

FIG. 3 provides prevalence of ERBB3 alterations by cancer-type. The figure was generated from the MSK-IMPACT dataset using www.cbioportal.org.

FIG. 4 sets out modeling of the location of various ERBB3 mutations understood to be involved in ligand binding and heterodimerization: ERBB3 domains I-III were separately super-imposed on the active EGFR dimer structure (PDB ID: 1IVO). Mutation positions M91 and R426 align with ligand-binding residues on EGFR and are therefore likely involved in ERBB3 ligand binding. Residue P262 aligns to the dimerization loop of EGFR and may therefore be involved in the homo or heterodimerization of ERBB3 in its active form. D297 appears to interact with the dimerization loop and may also be involved in the dimerization process. Residues R103, V104, A232 and M60 appear to be involved in the DI-DII interactions in the active form. G284 is proximate to the DI-DIII interface and may interact with DI upon mutation to R.

FIG. 5 sets out ERBB3 mutations on clinical sequencing of over 10,000 patients. (Zehir, Ahmet et al. Nature medicine, vol. 23, 6 (2017): 703-713). The figure was generated from the MSK-IMPACT dataset using www.cbioportal.org.

FIG. 6 lists the amino acid sequence of a) a common light chain amino; b) common light chain variable region DNA sequence and its translation (IGKV1-39/jk1). c) Common light chain constant region DNA sequence and translation. d) IGKV1-39/jk5 common light chain variable region translation. e) V-region IGKV1-39A; f) CDR1, CDR2 and CDR3 of a common light chain.

FIG. 7 lists the IgG heavy chains sequences for the generation of bispecific molecules. a) CH1 region. b) hinge region. c) CH2 region. d) CH3 domain containing variations L351K and T366K (KK). e) CH3 domain containing variations L351D and L368E (DE).

FIG. 8 lists the nucleic acid and amino acid sequences of the heavy chain of variable regions

### DETAILED DESCRIPTION OF THE INVENTION

The ErbB family of tyrosine kinase transmembrane receptors are also referred to as the human epidermal growth factor (EGF) receptor family (HER). The family has four members: ERBB (Erythroblastoma)-1, ERBB2, ERBB3 and ERBB4. The receptors (reviewed in Yarden and Pines 2012) are widely expressed on epithelial cells. Upregulation of HER receptors or their ligands, such as, neuregulin (NRG) (also known heregulin (HRG)) or epidermal growth factor (EGF), is a frequent event in human cancer (Wilson, Timothy R et al. Nature, vol. 487, 7408 (2012): 505-9). Overexpression of ERBB1 and ERBB2 in particular occurs in epithelial tumors and is associated with tumor invasion, metastasis, resistance to chemotherapy, and poor prognosis (Zhang, Hongtao et al. The Journal of clinical investigation, vol. 117, 8 (2007): 2051-8). In the normal breast, ERBB3 has been shown to be important in the growth and differentiation of luminal epithelium. For instance, loss/inhibition of ERBB3 results in selective expansion of the basal over the luminal epithelium (Balko, Justin M et al. Proceedings of the National Academy of Sciences, vol. 109, 1 (2012): 221-6). Binding of ligand to the extracellular domain of the RTKs induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the prosurvival pathway Akt (reviewed in Yarden, Yosef, and Gur Pines. Nature reviews. Cancer, vol. 12, 8 553-63). No specific endogenous ligand has been identified for ERBB2, which is therefore assumed to normally signal through heterodimerization (Sergina, Natalia V et al. Nature, vol. 445, 7126 (2007): 437-41). ERBB3 can be activated by engagement of its ligands. These ligands include but are not limited to neuregulin (NRG) (also known as heregulin (HRG)).

ERBB1 is known under various synonyms, the most common of which is EGFR. EGFR has an extracellular domain (ECD) that is composed of four sub-domains, two of which are involved in ligand binding and two of which are involved in homodimerization and heterodimerization. EGFR integrates extracellular signals from a variety of ligands to yield diverse intracellular responses. The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This receptor tyrosine kinase (RTK) has been extensively used as target for cancer therapy. Both small-molecule inhibitors targeting the RTK and monoclonal antibodies (mAbs) (monospecific bivalent) directed to the extracellular ligand-binding domains have been developed and have shown hitherto several clinical successes. The database accession number for the human EGFR protein and the gene encoding it is (GenBank NM_005228.3). This accession number is primarily given to provide a further method of identification of EGFR protein as a target, the actual sequence of the EGFR protein bound by an antibody may vary, for instance because of a mutation in the encoding gene such as those occurring in some cancers or the like.

The term ‘ERBB2’ as used herein refers to the protein that in humans is encoded by the ERBB2 gene. Alternative names for the gene or protein include CD340; HER2; HER-2/neu; MLN 19; NEU; NGL; TKR1. The ERBB2 gene is frequently called HER2 (from human epidermal growth factor receptor 2). Where reference is made herein to ERBB2, the reference refers to human ERBB2. An antibody comprising an antigen-binding site that binds ERBB2, binds human ERBB2. The ERBB2 antigen-binding site may, due to sequence and tertiary structure similarity between human and other mammalian orthologs, also bind such an ortholog but not necessarily so. Database accession numbers for the human ERBB2 protein and the gene encoding it are (NP_001005862.1, NP_004439.2 NC 000017.10 NT_010783.15 NC_018928.2). The accession numbers are primarily given to provide a further method of identification of ERBB2 as a target, the actual sequence of the ERBB2 protein bound the antibody may vary, for instance because of a mutation in the encoding gene such as those occurring in some cancers or the like. The ERBB2 antigen binding site binds ERBB2 and a variety of variants thereof, such as those expressed by some ERBB2 positive tumor cells. The antigen-binding site that binds ERBB2 preferably binds domain I of ERBB2.

The term ‘ERBB3’ as used herein refers to the protein that in humans is encoded by the ERBB3 gene. Alternative names for the gene or protein are HER3; LCCS2; MDA-BF-1; c-ERBB3; c-ERBB3; ERBB3-S; p180-ERBB3; p45-sERBB3; and p85-sERBB3. Where reference is made herein to ERBB3, the reference refers to human ERBB3. An antibody comprising an antigen-binding site that binds ERBB3, binds human ERBB3. The ERBB3 antigen-binding site may, due to sequence and tertiary structure similarity between human and other mammalian orthologs, also bind such an ortholog but not necessarily so. Database accession numbers for the human ERBB3 protein of the present disclosure and the gene encoding it are NP_001973.2 and NC_000012.11 which contains the genomic location of the ERBB3 gene on chromosome 12 (56473892 to 56497289). The accession numbers are primarily given to provide a further method of identification of ERBB3 as a target, the actual sequence of the ERBB3 protein bound by an antibody may vary, for instance because of a mutation in the encoding gene such as those occurring in some cancers or the like. The ERBB3 antigen binding site binds ERBB3 and a variety of variants thereof, such as those expressed by some ERBB3 positive tumor cells. The antigen-binding site that binds ERBB3 preferably binds domain III of ERBB3. Preferably, the protein sequence of the non-mutated or naturally-occurring human ERBB3 is the sequence of NP_001973.2, which is:

(SEQ ID No: 1)
MRANDALQVLGLLFSLARGSEVGNSQAVCPGTLNGLSVTGDAENQYQTL
YKLYERCEVVMGNLEIVLTGHNADLSFLQWIREVTGYVLVAMNEFSTLP
LPNLRVVRGTQVYDGKFAIFVMLNYNTNSSHALRQLRLTQLTEILSGGV
YIEKNDKLCHMDTIDWRDIVRDRDAEIVVKDNGRSCPPCHEVCKGRCWG
PGSEDCQTLTKTICAPQCNGHCFGPNPNQCCHDECAGGCSGPQDTDCFA
CRHFNDSGACVPRCPQPLVYNKLTFQLEPNPHTKYQYGGVCVASCPHNF
VVDQTSCVRACPPDKMEVDKNGLKMCEPCGGLCPKACEGTGSGSRFQTV
DSSNIDGFVNCTKILGNLDFLITGLNGDPWHKIPALDPEKLNVFRTVRE
ITGYLNIQSWPPHMHNFSVFSNLTTIGGRSLYNRGFSLLIMKNLNVTSL
GFRSLKEISAGRIYISANRQLCYHHSLNWTKVLRGPTEERLDIKHNRPR
RDCVAEGKVCDPLCSSGGCWGPGPGQCLSCRNYSRGGVCVTHCNFLNGE
PREFAHEAECFSCHPECQPMEGTATCNGSGSDTCAQCAHFRDGPHCVSS
CPHGVLGAKGPIYKYPDVQNECRPCHENCTQGCKGPELQDCLGQTLVLI
GKTHLTMALTVIAGLVVIFMMLGGTFLYWRGRRIQNKRAMRRYLERGES
IEPLDPSEKANKVLARIFKETELRKLKVLGSGVFGTVHKGVWIPEGESI
KIPVCIKVIEDKSGRQSFQAVTDHMLAIGSLDHAHIVRLLGLCPGSSLQ
LVTQYLPLGSLLDHVRQHRGALGPQLLLNWGVQIAKGMYYLEEHGMVHR
NLAARNVLLKSPSQVQVADFGVADLLPPDDKQLLYSEAKTPIKWMALES
IHFGKYTHQSDVWSYGVTVWELMTFGAEPYAGLRLAEVPDLLEKGERLA
QPQICTIDVYMVMVKCWMIDENIRPTFKELANEFTRMARDPPRYLVIKR
ESGPGIAPGPEPHGLTNKKLEEVELEPELDLDLDLEAEEDNLATTTLGS
ALSLPVGTLNRPRGSQSLLSPSSGYMPMNQGNLGESCQESAVSGSSERC
PRPVSLHPMPRGCLASESSEGHVTGSEAELQEKVSMCRSRSRSRSPRPR
GDSAYHSQRHSLLTPVTPLSPPGLEEEDVNGYVMPDTHLKGTPSSREGT
LSSVGLSSVLGTEEEDEDEEYEYMNRRRRHSPPHPPRPSSLEELGYEYM
DVGSDLSASLGSTQSCPLHPVPIMPTAGTTPDEDYEYMNRQRDGGGPGG
DYAAMGACPASEQGYEEMRAFQGPGHQAPHVHYARLKTLRSLEATDSAF
DNPDYWHSRLFPKANAQRT.

Preferably, the present disclosure includes an antibody that comprises an antigen binding site that can bind an extracellular part of ERBB2 and an antigen binding site that can bind an extracellular part of ERBB3 for use in a method of treatment of a cancer harboring a ERBB3 mutation in a subject in need thereof, wherein the ERBB3 mutation preferably comprises one or more mutations compared to the non-mutated sequence according to SEQ ID No: 1.

When reference is made to ERBB1, ERBB2 or ERBB3 or an alternative name for the same, the reference is to human ERBB1, ERBB2 or ERBB3. Antibodies as referred to herein bind to ERBB1, ERBB2 or ERBB3 and many mutated ERBB1, ERBB2 or ERBB3 proteins as can be found in cancers.

Cancer cells are known to harbor mutations in ERBB3 gene, resulting in amino acid substitutions at specific positions in the ERBB3 protein. These mutations treated by the method disclosed here preferably promote and/or are correlated with ligand independent heterodimerization of ERBB2 and ERBB3 and/or result in activation of the ERBB2 kinase domain through ERBB3 interaction, such that it promotes the activation of the ErbB signaling axis and downstream mitogen-activated protein kinase (MAPK) and the PI3K pathway signaling. Previously, it has been reported that a class of bispecific antibodies binding domain 1 of ERBB2 and domain 3 of ERBB3 are capable of arresting oncogenesis by binding domain 1 of ERBB2 and blocking the ligand for ERBB3 through binding domain 3 of ERBB3 (Geuijen et al. Cancer Cell 33, 922-936 (2018)). This mechanism disrupts the ligand-dependent heterodimerization of ERBB2 and ERBB3, and disrupts activation of the PI3K, mTOR pathway. It has been reported that mutations in ERBB3 may promote heterodimerization with ERBB2, activating the PI3K, mTOR pathway in a ligand independent fashion. Without being bound to any theory, it is believed that the present disclosure provides for a method of treatment of ERBB3 mutated cancer by use of a class of bispecific antibodies binding domain 1 of ERBB2 and binding domain 3 of ERBB3, not through blocking ligand interaction with ERBB3, but by sterically obstructing heterodimerization of ERBB2 and ERBB3, and preventing activation of the tyrosine kinase receptor of ERBB2 and activation of the PI3K and mTOR pathways. Thus, it is understood that the mechanism of action for the class of bispecific antibodies binding domain 1 of ERBB2 and domain 3 of ERBB3, including preferably MF3958×MF3178 for inhibiting ligand independent ERBB3 oncogenesis differs from the mechanism of action for these bispecific antibodies ability to inhibit ligand correlated ERBB2, ERBB3 heterodimerization and oncogenesis.

Preferably, the mutations involved in ligand-independent heterodimerization involve the amino acids at position V104, A232, P262, G284, Q809, 5846 and E928. Preferably, the mutations are V104L or M, A232V, P262H, L or S, G284R, Q809R, S846I or E928G.

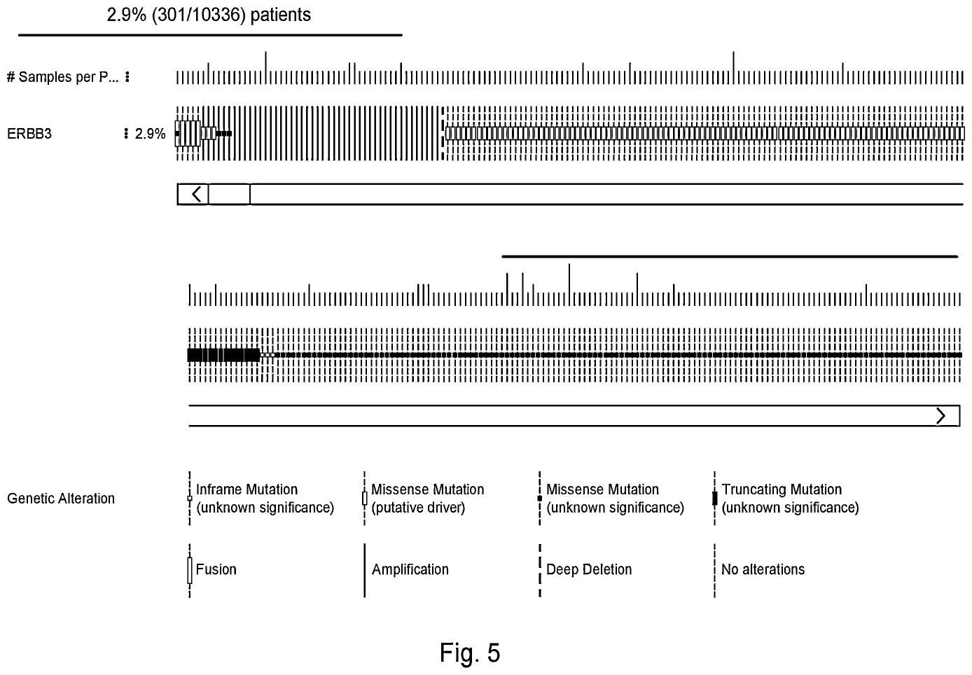

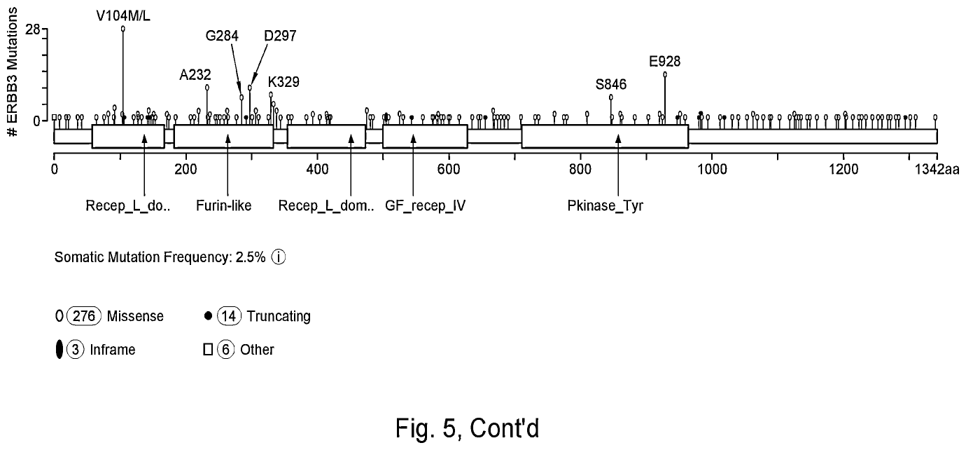

The ERBB3 mutation preferably occurs in the extracellular domain of ERBB3, preferably in domains DI-DIV, where domain I comprises residues 56-166, domain II comprises residues 182-332, domain III comprises residues 353-472 and domain IV comprises residues 499-629 (FIG. 5).

More preferably the mutation occurs at positions involved in active formation of ERBB3 receptor-ligand complex and/or homo/heterodimerization of ERBB3, comprising positions M60, M91, R103, A232, P262, G284, and D297.

Without being bound by theory, it is believed that prevention of heterodimerization via binding of the bispecific antibody of the present disclosure to domain 1 of ERRB2 and domain 3 of ERRB3, makes an ERRB3 oncogenic driver mutation present in the intracellular domain obstructed from contributing to activation of the tyrosine kinase domain of ERRB2, rendering a subject with such a ERRB3 mutation to be amenable for treatment.

In another aspect of the invention, the ERBB3 mutation comprises a mutation in the intracellular tyrosine kinase domain of ERBB3 comprising amino acid residues 710-964, preferably at positions Q809, S846, Q865 or E928. Several mutations occurring in the intracellular kinase domain, such as at position S846 and Q809, have been reported to cause stabilization of the ERBB2/ERBB3 heterodimer. The present disclosure provides for a method of treatment with a bispecific antibody binding to domain 1 of ERRB2 and domain 3 of ERRB3, whereby an oncogenic driver mutation present in the ERRB3 intracellular domain is obstructed from contributing to activation of the tyrosine kinase domain of ERRB2, rendering a subject with such a ERRB3 mutation to be amenable for treatment. In a preferred embodiment, the ERBB3 mutation is a mutation at positions Q809 or S846.

In some aspect, the ERBB3 mutation comprises a mutation that is oncogenic driver, comprising amino acid residues M60, M91, R103, V104, R135, F219, H228, A232, P262, G284, D297, K329, E332, T355, R475, Q809, S846, Q865 or E928. More preferably the ERBB3 mutations are hotspot mutations comprising V104, A232, G284, D297, K329, T355, S846 or E928.

Specifically, the ERBB3 mutation, or cancer comprising said mutation, comprises any one of the following:
M60N, where N is any naturally occurring amino acid, preferably K;
M91N, where N is any naturally occurring amino acid, preferably I;
R103N, where N is any naturally occurring amino acid, preferably G;
V104N, where N is any naturally occurring amino acid, preferably L or M;
R135N, where N is any naturally occurring amino acid, preferably C;
F219N, where N is any naturally occurring amino acid, preferably L;
H228N, where N is any naturally occurring amino acid, preferably Q;
A232N, where N is any naturally occurring amino acid, preferably V;
P262N, where N is any naturally occurring amino acid, preferably H or L or S;
G284N, where N is any naturally occurring amino acid, preferably R;
D297N, where N is any naturally occurring amino acid, preferably Y or A or H or N or V;
K329N, where N is any naturally occurring amino acid, preferably E or I or T;
E332N, where N is any naturally occurring amino acid, preferably K;
T355N, where N is any naturally occurring amino acid, preferably A or I or P;
R475N, where N is any naturally occurring amino acid, preferably W;
Q809N, where N is any naturally occurring amino acid, preferably R;
S846N, where N is any naturally occurring amino acid, preferably I;
Q865N, where N is any naturally occurring amino acid, preferably H;
E928N, where N is any naturally occurring amino acid, preferably G.

In these situations, N is an amino acid of the ERRB3 protein at the indicated position which is different from the naturally occurring, non-mutated amino acid residue of corresponding position. In an aspect of the disclosure, ERBB3 mutations at position R426 are excluded.

Preferably cancer cells harboring the ERBB3 mutation lack another oncogenic driver, including, but not limited to KRAS, NRAS, PIK3CA, BRAF or the cell shows absence of oncogenic mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, and PTEN.

Preferably cancer cells harboring the ERBB3 mutation lack the following oncogenic amplifications c-MET amplification, c-MYC amplification, EGFR amplification, ERBB2 amplification, and MDM2 amplification.

Preferably cancer cells harboring the ERBB3 mutation do not have PTEN loss. PTEN (HGNC identifier 9588) is a phosphatase which metabolizes PIP3, the lipid product of PI 3-Kinase, directly opposing the activation of the oncogenic PI3K/AKT/mTOR signaling network. Loss of function of the PTEN tumor suppressor is a common event observed in cancer types. With ‘PTEN loss’ is meant loss of function of the PTEN tumor suppressor.

Any of the ERBB3 mutations described above promote and/or are correlated with ligand independent heterodimerization of ERBB2 and ERBB3 and/or result in activation of the ERBB2 kinase domain through ERBB3 interaction, such that the mutation(s) promotes the activation of the ErbB signaling axis and downstream mitogen-activated protein kinase (MAPK) and the PI3K pathway signaling.

The invention further provides a bispecific antibody that comprises a first antigen-binding site that binds an extracellular part of ERBB2 and a second antigen-binding site that binds an extracellular part of ERBB3 for use in the treatment of a subject that is at risk of having an ERBB2 and ERBB3 positive cancer, cells of which cancer comprise an ERBB3 mutation, and which subject is a pretreatment cancer subject that has received a previous ERBB1; ERBB2 or ERBB3 targeted tumor treatment.

Further provided is a method of treating a subject that is at risk of having an ERBB2 and ERBB3 positive cancer, cells of which cancer comprise an ERBB3 mutation, and which subject is a pretreatment cancer subject that has received a previous treatment of chemotherapy or ERBB2 or ERBB3 targeted tumor treatment, the method comprising administering to the subject in need thereof a bispecific antibody that comprises a first antigen-binding site that binds an extracellular part of ERBB2 and a second antigen-binding site that binds an extracellular part of ERBB3.

The chemotherapy according to the present invention preferably comprises gemcitabine, capecitabine, carboplatin, a taxane, such as docetaxel or paclitaxel, 5-fluorouracil (with or without radiotherapy), vinorelbine, mitoxantrone, vinblastine, cisplatin (or pemetrexed), oxaliplatin, carboplatin, ifosfamide, mytomycine C, vindesine, etoposide, Folfox (i.e. a combination of 5-fluorouracil, leucovorin, and oxaliplatin) or Folfiri (i.e. a combination of leucovorin, 5-fluorouracil and irinotecan), Folfirinox (a combination of leucovorin, 5-fluorouracil, irinotecan and oxaliplatin) or any combination thereof.

The subject has preferably undergone chemotherapy or a therapy targeted towards ERBB2 inhibition. Inhibition of ERBB2 signaling also referred to as ERBB2 targeted tumor treatment preferably comprises administration of a monospecific bivalent antibody comprising antigen-binding sites that bind an extracellular part of ERBB2; or administration of an ERBB2 tyrosine kinase inhibitor (TKI) or a combination thereof, and wherein the monospecific bivalent antibody is preferably trastuzumab, pertuzumab, or trastuzumab-emtansine and wherein the ERBB2 TKI is preferably one or more of lapatinib, canertinib, neratinib, tucatinib (irbinitinib), CP-724714, tarloxitinib, mubritinib, afatinib, varlitinib, and dacomitinib, preferably lapatinib, canertinib, neratinib, tucatinib (irbinitinib), mubritinib, afatinib, varlitinib, or dacomitinib, more preferably afatinib.

Accordingly to the present disclosure the subject has alternatively be treated with an ERBB3 targeted tumor treatment preferably comprises administration of a monospecific bivalent antibody comprising antigen-binding sites that bind an extracellular part of ERBB3; or administration of an ERBB3 tyrosine kinase inhibitor (TKI) or a combination thereof, and wherein the monospecific bivalent antibody is preferably patritumab, seribantumab, lumretuzumab, elgemtumab, GSK2849330, KTN3379 or AV-203.

A method of treatment or bispecific antibody for use in the treatment as described in the present disclosure preferably comprises further determining whether the cell comprises an ERBB3 mutation or whether the tumor comprises cells with an ERBB3 mutation. This can for instance be done on cells of a biopsy. Various methods are available and many are known in the art. One way is by means of PCR-amplification with primers that span the region of the ERBB3 mutation. This can be implemented for ERBB3 mutations that are known to occur. New mutations can also be detected readily through techniques known to those of ordinary skill in the art, including by next-generation DNA or RNA sequencing.

Techniques available to identify putative ERBB3 mutations include Allele specific RNA based methodology including RT-PCR, Real-time PCR, Transcriptome analysis, Anchored multiplex PCR, nCounter, FISH, DNA-based methodologies including Hybrid capture-based next generation sequencing (NGS), Amplicon-based NGS, among other techniques available commercially.

The cancer harboring an ERBB3 mutation includes colorectal cancer, gastric, non-small-cell lung (NSCLC) adenocarcinoma (adeno), NSCLC (squamous), renal carcinoma, melanoma, ovarian, lung large cell, esophageal, small-cell lung cancer, hepatocellular (HCC), breast cancer, hormone-positive breast cancer, glioblastoma, and head and neck cancer.

The cancer harboring an ERBB3 mutation is preferably a gastric cancer such as a gastric adenocarcinoma or an esophago-gastric cancer, a pancreatic cancer, a pancreatic ductal adenocarcinoma, a sarcoma, a bladder cancer, a colorectal cancer, a gallbladder cancer, head and neck cancer, a prostate cancer, uterine/endometrial cancer, a breast cancer, an ovarian cancer, a liver cancer, a lung cancer such as non-small cell lung cancer, preferably a non-small cell lung cancer, more preferably invasive mucinous adenocarcinoma. More preferably, the cancer or tumor comprises mutation A232V in the ERBB3 protein. More preferably the cancer or tumor is bladder cancer comprising mutation A232V in the ERBB3 protein.

The cancer or tumor preferably comprises mutation V104M in the ERBB3 protein. More preferably, the cancer or tumor is ovarian cancer, such as clear cell carcinoma comprising mutation V104M in the ERBB3 protein.

Various methods are available to determine the level of ErbB receptors on a cell of a cancer. Examples are immunohistochemistry or fluorescence in situ hybridization. The HercepTest™ and/or HER2 FISH (pharm Dx™), marketed both by Dako Denmark A/S, and/or using a HERmark® assay, marketed by Monogram Biosciences are examples of suitable assays for determining ERBB2 or ERBB3 cell surface receptor density. Other methods for determining the ERBB2 receptor cell density are well-known to a skilled person. In vivo methods for determining ERBB2 are also known, see, e.g., Chernomoridik et al. Mol Imaging. 2010 August; 9(4): 192-200 and Ardeshirpour et al. Technol Cancer Res Treat. 2014 October; 13(5): 427-434. Preferably, the methods disclosed herein further comprise determining the ERBB2 cell-surface receptor density for said cell or tumor. Such methods are known to a skilled person (see, e.g., van der Woning and van Zoelen Biochem Biophys Res Commun. 2009 Jan. 9; 378(2):285-9). Preferably, the methods disclosed herein further comprise determining the ERBB1 cell-surface receptor density for said cell or tumor. Such methods are known to a skilled person (see, e.g., EGFR pharmDxTMKit (Dako)) and McDonagh et al. Mol Cancer Ther 2012; 11:582). Similar methods can be used to determine ERBB4 cell-surface receptor density.

In some embodiments, the ERBB1, ERBB2, ERBB3, and ERBB4 cell-surface receptor densities are determined by FACS analysis on biopsied tumor cells.

The amount of bispecific antibody to be administered to a subject is typically in the therapeutic window, meaning that a sufficient quantity is used for obtaining a therapeutic effect, while the amount does not exceed a threshold value leading to an unacceptable extent of side-effects. The selected dosage level will depend upon a variety of factors including the route of administration, the time of administration, the rate of excretion of the particular compound being employed, the duration of the treatment, other drugs, compounds and/or materials used in combination, the age, sex, weight, condition, general health and prior medical history of the subject being treated, and like factors well known in the medical arts. The dosage is in the range of 200-1000 mg, weekly, biweekly or tri-weekly. Preferably, dosing the therapeutic of the present disclosure, targeting ERBB2×ERBB3, follows a weekly, biweekly or tri-weekly administration regimen of 750 mg, preferably a bi-weekly or tri-weekly dose of 750 mg. The dosing is preferably in subjects with cancer having a solid tumor harboring an ERBB3 mutation, following, a dosing regimen is comprising a weekly flat dose administration of 400 mg, preferably commenced after a single administration of 800 mg. Following this alternative dosing regimen, the bispecific antibody of the invention is preferably administered in a weekly dose of 400 mg for 3 weeks followed by 1 week without dosing. Next, one or more cycles of a period of four weeks, consisting of three weekly flat dosages of 400 mg, followed by a week without administration is followed. This is preferably followed until a therapeutic effect is observed. A dosing regimen of the present disclosure comprises a bi-weekly cycle with a flat dose of 750 mg weekly commenced after an initial administration of a 750 mg infusion over a four-hour period, followed by a biweekly two-hour infusion of 750 mg in a four-week cycle. This is preferably followed until a therapeutic effect is observed.

Dosing preferably involves intravenous injections of two infusions of the bispecific antibody of the invention to arrive at the complete dose, preferably when dosing >360 mg antibody. Alternatively, a single infusion of the complete dose may be given for lower dosages, for instance when dosing ≤360 mg antibody. Pre-medication may be included in the dosing regimen to mitigate infusion-related reactions.

Preferably, treatment comprises stabilization of the tumor in terms of size or lesions or prevention of further tumor growth, including tumor reduction. Preferably, treatment or administration is with the bispecific antibody according to the invention on a weekly regimen and proceeds for a period of at least 1, 2, 4, 8 or at least 12 months. Preferably, a dosing regimen is followed comprising a weekly cycle with a flat dose of 400 mg weekly commenced after an initial administration of 800 mg. From week 3, the bispecific antibody of the invention is given at a weekly dose of 400 mg for 3 weeks followed by 1 week without dosing of the bispecific antibody of the invention. Alternatively, a dosing regimen is followed comprising a bi-weekly cycle with a flat dose of 750 mg weekly commenced after an initial administration of a 750 mg infusion over a four-hour period, followed by a bi-weekly two-hour infusion of 750 mg in a four-week cycle. A further alternative comprises a tri-weekly administration of a flat dose of 750 mg per subject.

Preferably, diagnosis involves molecular profiling using a targeted sequencing method, analysis of at last one biomarker, including fusions, insertion/deletions (indels), single nucleotide variants and/or copy number variations. Preferably, the tumor genome harboring ERBB3 mutations lack another oncogenic driver, including, but not limited to KRAS, NRAS, PIK3CA, BRAF or the tumor cell shows absence of oncogenic mutations in one or more genes selected from the group consisting of BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, and PTEN.

Preferably the tumor genome harboring ERBB3 mutations lack the following oncogenic amplifications c-MET amplification, c-MYC amplification, EGFR amplification, ERBB2 amplification, MDM2 amplification.

Preferably the tumor genome harboring ERBB3 mutations do not have PTEN loss.

Preferably, disease progression comprises a measuring of anti-tumor activity by using a CT-scan and assessment by RECIST v1.1 determining objective overall response rate (ORR), duration of response (DOR), progression-free survival (PFS) and survival. Preferably, a bispecific antibody that has a first antigen-binding site that binds an extracellular part of ERBB2 and a second antigen-binding site that binds an extracellular part of ERBB3, in particular MF3958×MF3178, stabilizes tumors in terms of size or lesions or the treatment prevents further tumor growth. Preferably, treatment is without drug related toxicity or has a good safety profile with limited occurrence of grade 3-5 adverse events that are actual or suspected to be drug related.

The bispecific antibodies can be formulated as a pharmaceutical composition comprising a pharmaceutically acceptable carrier, diluent, or excipient, and additional, optional, active agents. The antibodies and compositions comprising the antibodies can be administered by any route including parenteral, enteral, and topical administration. Parenteral administration is usually by injection, and includes, e.g., intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, intratumoral, and intrasternal injection and infusion.

The disclosure provides bispecific antibodies for use in the methods and treatments described herein. Suitable bispecific antibodies comprise a first antigen-binding site that binds ERBB2 and a second antigen-binding site that binds ERBB3. The bispecific antibody reduces or can reduce a ligand-induced receptor function of ERBB3 on an ERBB2 and ERBB3 positive cell and/or disrupt ERBB2 and ERBB3 heterodimerization. Preferred antibodies and their preparation are disclosed in WO 2015/130173, which is hereby incorporated by reference. The examples in WO 2015/130173 further describe a number of properties of the antibodies, such as ligand binding and epitope mapping.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence.

The therapeutic amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

A therapeutic amount may vary according to factors such as the disease state, age, sex, and weight of the individual to be treated, and the ability of the agent or combination of agents to elicit a desired response in the individual.

A therapeutic amount can be administered in one or more administrations.

A therapeutic amount also includes an amount that balances any toxic or detrimental effects of the agent or combination of agents and the therapeutically beneficial effects.”

As used herein, the term “antigen-binding site” refers to a site derived from and preferably as present on a bispecific antibody which is capable of binding to antigen. An antigen-binding site is typically formed by and present in the variable domain of the antibody. The variable domain contains said antigen-binding site. The antigen-binding site can bind to the antigen under normal physiological conditions. This is often also referred to as that the antigen binding site “binds” the antigen.

In one embodiment an antibody variable domain comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

As used herein, antigen-binding refers to the typical binding capacity of an antibody to its antigen. An antibody comprising an antigen-binding site that binds to ERBB2, binds to ERBB2 and, under otherwise identical conditions, at least 100-fold lower to the homologous receptors ERBB1 and ERBB4 of the same species. An antibody comprising an antigen-binding site that binds to ERBB3, binds to ERBB3 and, under otherwise identical conditions, not to the homologous receptors ERBB1 and ERBB4 of the same species.

Considering that the ErbB-family is a family of cell surface receptors, the binding is typically assessed on cells that express the receptor(s). Binding of an antibody to an antigen can be assessed in various ways. One way is to incubate the antibody with the antigen (preferably cells expressing the antigen), removing unbound antibody (preferably by a wash step) and detecting bound antibody by means of a labeled antibody that binds to the bound antibody.

Antigen binding by an antibody is typically mediated through the complementarity regions of the antibody and the specific three-dimensional structure of both the antigen and the variable domain allowing these two structures to bind together with precision (an interaction similar to a lock and key), as opposed to random, non-specific sticking of antibodies. As an antibody typically recognizes an epitope of an antigen, and as such epitope may be present in other compounds as well, antibodies according to the present invention that bind ERBB2 and/or ERBB3 may recognize other proteins as well, if such other compounds contain the same epitope. Hence, the term “binding” does not exclude binding of the antibodies to another protein or protein(s) that contain the same epitope. Such other protein(s) is preferably not a human protein. An ERBB2 antigen-binding site and an ERBB3 antigen-binding site as defined herein typically do not bind to other proteins on the membrane of cells in a post-natal, preferably adult human.

The term “interferes with binding” as used herein means that the antibody is directed to an epitope on ERBB3 and the antibody competes with ligand for binding to ERBB3. The antibody may diminish ligand binding, displace ligand when this is already bound to ERBB3 or it may, for instance through steric hindrance, at least partially prevent that ligand can bind to ERBB3.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Antibodies such the bispecific antibodies of the present invention comprise the constant domains (Fc part) of a natural antibody. An antibody of the invention is typically a bispecific full length antibody, preferably of the human IgG subclass. Preferably, an antibody as disclosed herein is of the human IgG1 subclass. Such antibodies have good ADCC properties, have favorable half-life upon in vivo administration to humans and CH3 engineering technology exists that can provide for modified heavy chains that preferentially form heterodimers over homodimers upon co-expression in clonal cells.

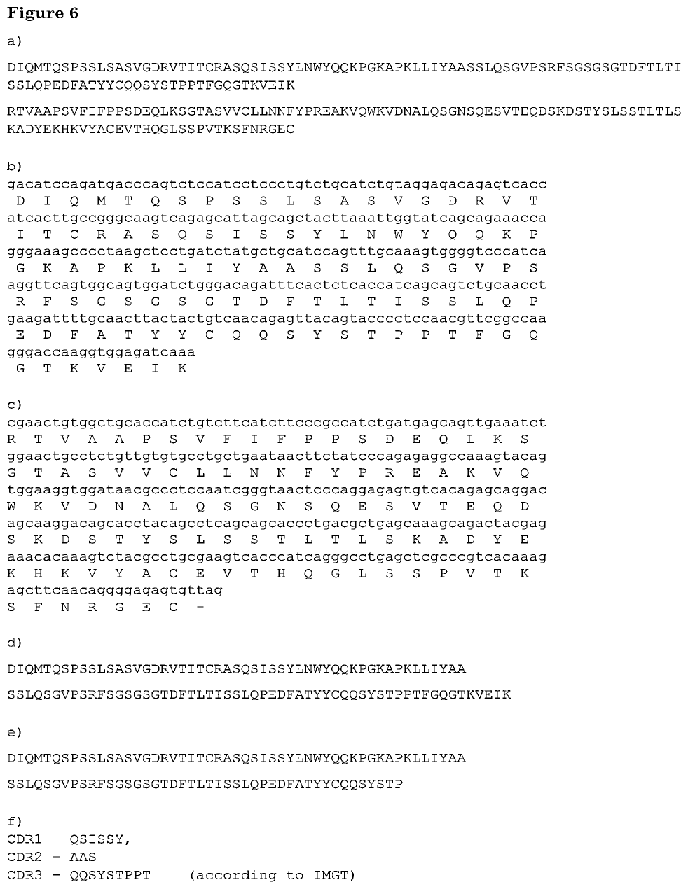

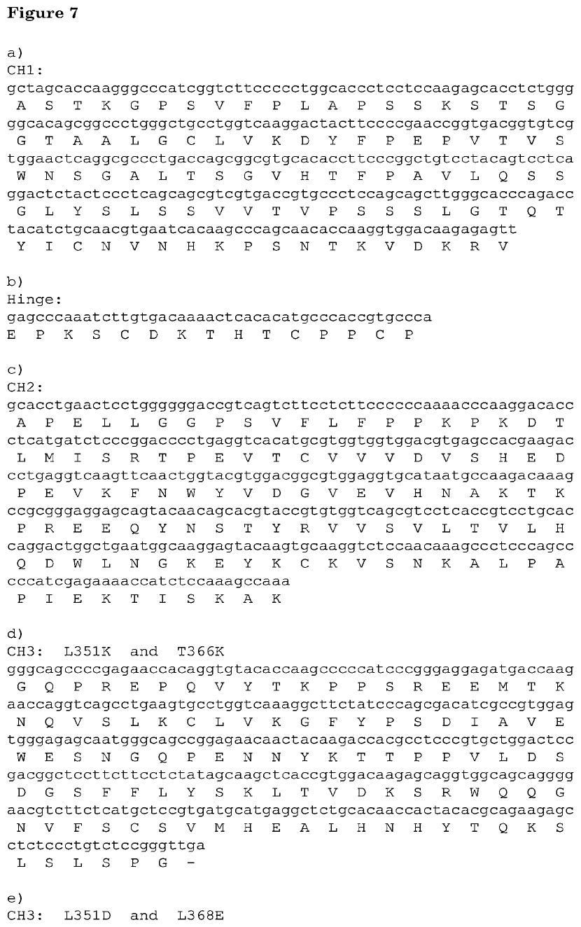

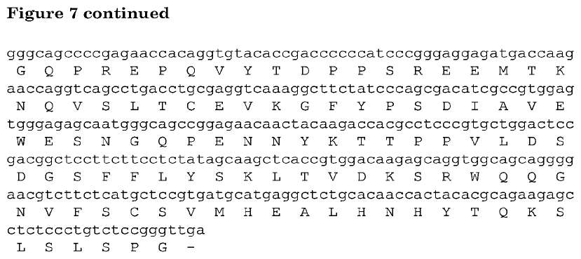

An antibody as disclosed herein is preferably a “full length” antibody. The term ‘full length’ is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL (suitable amino acid sequences for the respective domains are depicted in FIG. 6 and FIG. 7. An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489).

Full length IgG antibodies are preferred because of their favorable half-life and the need to stay as close to fully autologous (human) molecules for reasons of immunogenicity. An antibody as disclosed herein is preferably a bispecific IgG antibody, preferably a bispecific full length IgG1 antibody. IgG1 is favored based on its long circulatory half-life in man. In order to prevent any immunogenicity in humans it is preferred that the bispecific IgG antibody is a human IgG1.

The term ‘bispecific’ (bs) means that one part of the antibody (as defined above) binds to one epitope on an antigen whereas a second part binds to a different epitope. The different epitope is typically present on a different antigen. The heavy chain variable regions of the bispecific antibody are typically different from each other, whereas the light chain variable regions are preferably the same. A bispecific antibody wherein the different heavy chain variable regions are associated with the same, or a common, light chain is also referred to as a bispecific antibody with a common light chain. A bispecific antibody as described herein typically comprises one variable domain that binds ERBB2 and another variable domain that binds ERBB3.

Preferred bispecific antibodies can be obtained by co-expression of two different heavy chains and a common light chain in a single cell. When wildtype CH3 domains are used, co-expression of two different heavy chains and a common light chain will result in three different species, AA, AB and BB. To increase the percentage of the desired bispecific product (AB) CH3 engineering can be employed, or in other words, one can use heavy chains with compatible heterodimerization domains, as defined hereunder. Suitable compatible CH3 heterodimerization domains are depicted in FIGS. 7d and 7e.

The term ‘compatible heterodimerization domains’ as used herein refers to protein domains that are engineered such that engineered domain A′ will preferentially form heterodimers with engineered domain B′ and vice versa, whereas homodimerization between A′-A′ and B′-B′ is diminished.

The term ‘common light chain’ refers to light chains which may be identical or have some amino acid sequence differences while the binding specificity of the full length antibody is not affected. It is for instance possible, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common VL’, ‘single light chain’, ‘single VL’, with or without the addition of the term ‘rearranged’ are all used herein interchangeably.

A common light chain (variable region) preferably has a germline sequence. A preferred germline sequence is a light chain variable region that is frequently used in the human repertoire and has good thermodynamic stability, yield and solubility. In a preferred embodiment the light chain comprises a light chain region comprising the amino acid sequence of an IgVκ1-39*01 gene segment as depicted FIG. 6, more-preferably common light chain IGKV1-39/jk1 with 0-10, preferably 0-5 amino acid insertions, deletions, substitutions, additions or a combination thereof. IgVκ1-39 is short for Immunoglobulin Variable Kappa 1-39 Gene. The gene is also known as Immunoglobulin Kappa Variable 1-39; IGKV139; or IGKV1-39. External Ids for the gene are HGNC: 5740; Entrez Gene: 28930; Ensembl: ENSG00000242371. The variable region of IGKV1-39 is listed in the FIG. 6. The V-region can be combined with one of five J-regions. FIG. 6 describes two preferred sequences for IgVκ1-39 in combination with a J-region. The joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org).

It is preferred that the IgVκ1-39*01 comprising light chain variable region is a germline sequence. It is further preferred that the IGJκ1*01 or/IGJκ5*01 comprising light chain variable region is a germline sequence. In a preferred embodiment, the IGKV1-39/jk1 or IGKV1-39/jk5 light chain variable regions are germline sequences.

In a preferred embodiment the light chain variable region comprises a germline IgVκ1-39*01. In a preferred embodiment the light chain variable region comprises the kappa light chain IgVκ1-39*01/IGJκ1*01 or IgVκ1-39*01/IGJκ5*01. In a preferred embodiment a IgVκ1-39*01/IGJκ1*01. The light chain variable region preferably comprises a germline kappa light chain IgVκ1-39*01/IGJκ1*01 or germline kappa light chain IgVκ1-39*01/IGJκ5*01, preferably a germline IgVκ1-39*01/IGJκ1*01.

Those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. The light chain can also be a light chain as specified herein above, having 1-5 amino acid insertions, deletions, substitutions or a combination thereof.

Preferably, both the first antigen binding site and said second antigen binding site comprise a light chain variable region comprising a CDR1 having the sequence (RASQSISSYLN) (SEQ ID NO:2), a CDR2 having the sequence (AASSLQS) (SEQ ID NO: 3), and a CDR3 having the sequence (QQSYSTPPT) (SEQ ID NO: 4).

Antibodies disclosed herein can reduce a ligand-induced receptor function of ERBB3 on an ERBB2 and ERBB3 positive cell. In the presence of excess ERBB2, ERBB2/ERBB3 heterodimers may provide a growth signal to the expressing cell in the absence of detectable ligand for the ERBB3 chain in the heterodimer. This ERBB3 receptor function is herein referred as a ligand-independent receptor function of ERBB3. The ERBB2/ERBB3 heterodimer also provide a growth signal to the expressing cell in the presence of an ERBB3 ligand. This ERBB3 receptor function is herein referred to as a ligand-induced receptor function of ERBB3.

The term “ERBB3 ligand” as used herein refers to polypeptides which bind and activate ERBB3. Examples of ERBB3 ligands include, but are not limited to neuregulin 1 (NRG) and neuregulin 2, betacellulin, heparin-binding epidermal growth factor, and epiregulin. The term includes biologically active fragments and/or variants of a naturally occurring polypeptide.

Preferably, the ligand-induced receptor function of ERBB3 is ERBB3 ligand-induced growth of an ERBB2 and ERBB3 positive cell. In a preferred embodiment said cell is an MCF-7 cell (ATCC® HTB-22™); an SKBR3 (ATCC® HTB-30™) cell; an NCI-87 (ATCC® CRL-5822™) cell; a BxPC-3-luc2 cell (Perkin Elmer 125058), a BT-474 cell (ATCC® HTB-20™) or a JIMT 1 cell (DSMZ no.: ACC 589).

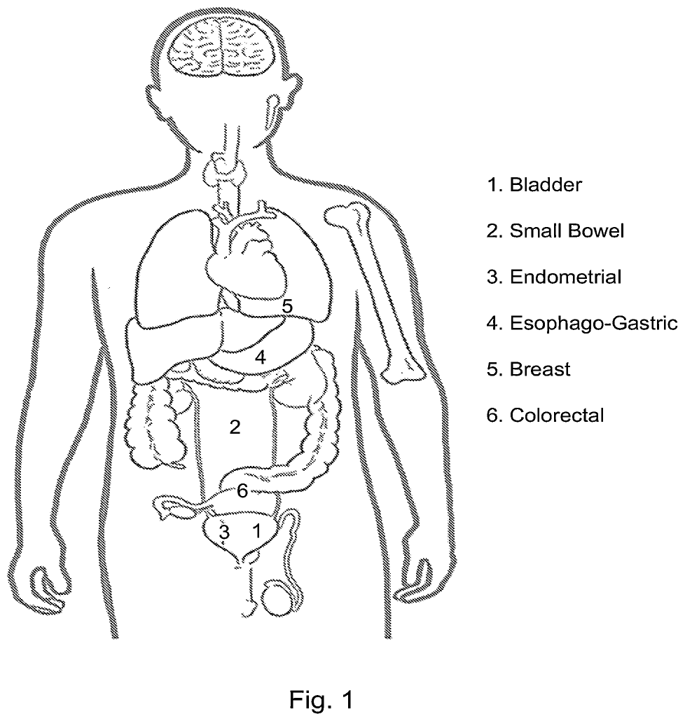

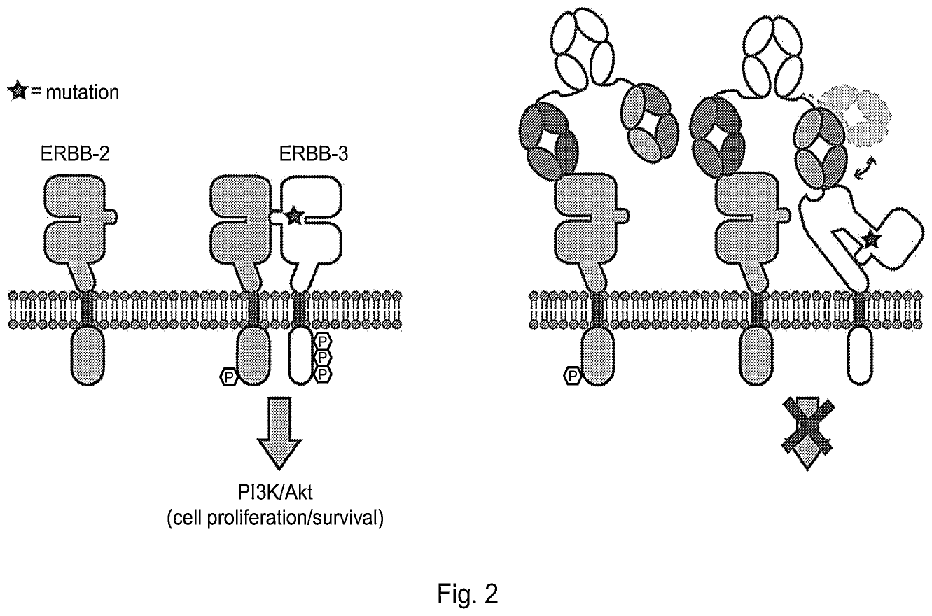

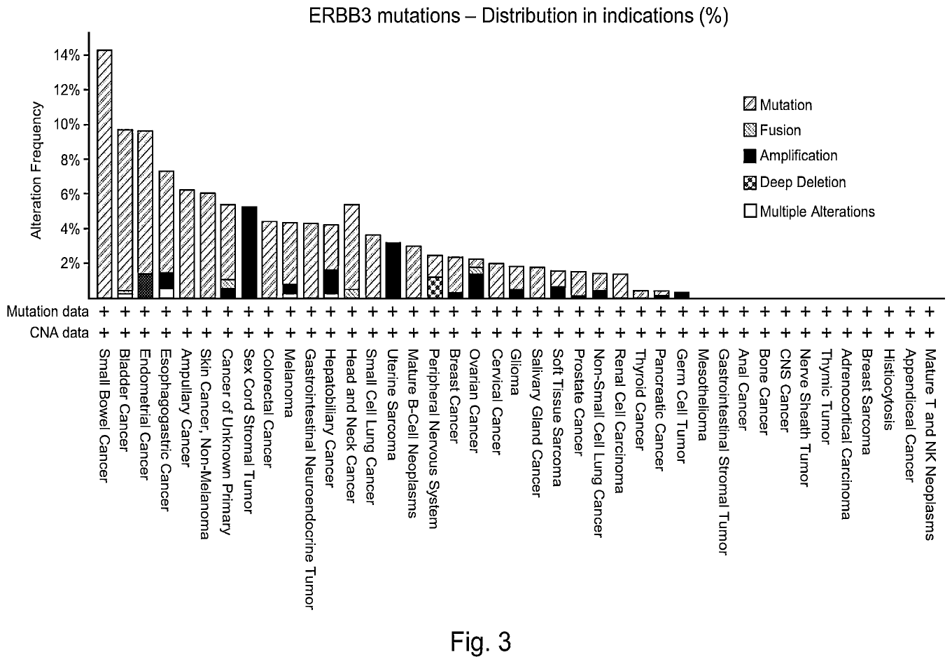

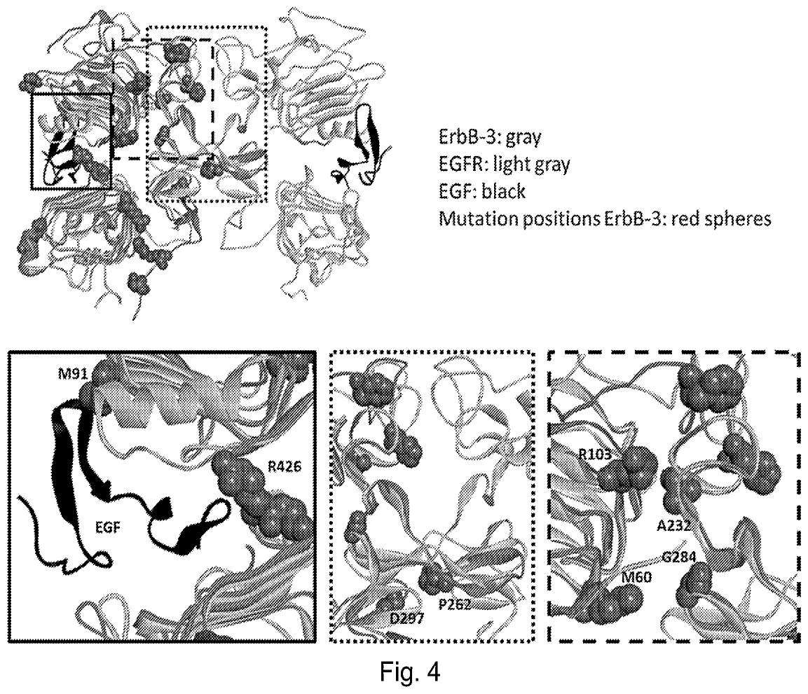

The ERBB2 protein contains several domains (see for reference FIG. 1 of Landgraf, R Breast Cancer Res. 2007; 9(1): 202-). The extracellular domains are referred to as domains I-IV. The place of binding to the respective domains of antigen-binding sites of antibodies described herein has been mapped. A bispecific antibody with an antigen-binding site (first antigen-binding site) that binds domain I or domain IV of ERBB2 (first antigen-binding site) comprises a heavy chain variable region that maintains significant binding specificity and affinity for ERBB2 when combined with various light chains. Bispecific antibodies with an antigen-binding site (first antigen-binding site) that binds domain I or domain IV of ERBB2 (first antigen-binding site) and an antigen-binding site for ERBB3 (second antigen-binding site) are more effective in reducing a ligand-induced receptor function of ERBB3 when compared to a bispecific antibody comprising an antigen-binding site (first antigen-binding site) that binds to another extra-cellular domain of ERBB2. A bispecific antibody comprising an antigen-binding site (first antigen-binding site) that binds ERBB2, wherein said antigen-binding site binds to domain I or domain IV of ERBB2 is preferred. Preferably said antigen-binding site binds to domain IV of ERBB2. Preferred antibodies comprises a first antigen-binding site that binds domain I of ERBB2 and a second antigen-binding site that binds domain III of ERBB3.

In one preferred embodiment, said antibody comprises an antigen-binding site that binds at least one amino acid of domain I of ERBB2 selected from the group consisting of T144, T164, R166, P172, G179, 5180 and R181, and surface-exposed amino acid residues that are located within about 5 amino acid positions from T144, T164, R166, P172, G179, S180 or R181.

In one preferred embodiment, said antibody preferably comprises an antigen-binding site that binds at least one amino acid of domain III of ERBB3 selected from the group comprising R426 and surface-exposed amino acid residues that are located within 11.2 Å from R426 in the native ERBB3 protein.

A bispecific antibody with an antigen-binding site (first antigen-binding site) that binds ERBB2, and that further comprises ADCC are more effective than other ERBB2 binding antibodies that did not have significant ADCC activity, particularly in vivo. A bispecific antibody which exhibits ADCC is therefore preferred. By engineering Fc regions (through introducing amino acid substitutions) that bind to activating receptors with greater selectivity, antibodies can be created that have greater capability to mediate cytotoxic activities desired by an anti-cancer Mab.

One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). Further provided is therefore a bispecific antibody as disclosed herein, which is afucosylated. Alternatively, or additionally, multiple other strategies can be used to achieve ADCC enhancement, for instance including glycoengineering and mutagenesis, all of which seek to improve Fc binding to low-affinity activating FcγRIIIa, and/or to reduce binding to the low affinity inhibitory FcγRIIb.

Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are typically co-incubated with the antibody-labeled target cells. Target cell lysis is subsequently typically measured by release of intracellular label, for instance by a scintillation counter or spectrophotometry.

In preferred bispecific antibodies, the affinity of said second antigen-binding site for an ERBB3 positive cell is equal to, or preferably higher than, the affinity of said first antigen-binding site for an ERBB2 positive cell. The affinity (KD) of said second antigen-binding site for an ERBB3 positive cell is preferably lower than or equal to 2.0 nM, more preferably lower than or equal to 1.5 nM, more preferably lower than or equal to 1.39 nM, more preferably lower than or equal to 0.99 nM. In one preferred embodiment, the affinity of said second antigen-binding site for ERBB3 on SK BR 3 cells is lower than or equal to 2.0 nM, more preferably lower than or equal to 1.5 nM, more preferably lower than or equal to 1.39 nM, preferably lower than or equal to 0.99 nM. In one embodiment, said affinity is within the range of 1.39-0.59 nM. In one preferred embodiment, the affinity of said second antigen-binding site for ERBB3 on BT 474 cells is lower than or equal to 2.0 nM, more preferably lower than or equal to 1.5 nM, more preferably lower than or equal to 1.0 nM, more preferably lower than 0.5 nM, more preferably lower than or equal to 0.31 nM, more preferably lower than or equal to 0.23 nM. In one embodiment, said affinity is within the range of 0.31-0.15 nM. The above-mentioned affinities are preferably as measured using steady state cell affinity measurements, wherein cells are incubated at 4° C. using radioactively labeled antibody, where after cell-bound radioactivity is measured, as described in the Examples of WO 2015/130173.

The affinity (KD) of said first antigen-binding site for an ERBB2 positive cell is preferably lower than or equal to 5.0 nM, more preferably lower than or equal to 4.5 nM, more preferably lower than or equal to 3.9 nM. In one preferred embodiment, the affinity of said first antigen-binding site for ERBB2 on SK BR 3 cells is lower than or equal to 5.0 nM, preferably lower than or equal to 4.5 nM, more preferably lower than or equal to 4.0 nM, more preferably lower than or equal to 3.5 nM, more preferably lower than or equal to 3.0 nM, more preferably lower than or equal to 2.3 nM. In one embodiment, said affinity is within the range of 3.0-1.6 nM. In one preferred embodiment, the affinity of said first antigen-binding site for ERBB2 on BT 474 cells is lower than or equal to 5.0 nM, preferably lower than or equal to 4.5 nM, more preferably lower than or equal to 3.9 nM. In one embodiment, said affinity is within the range of 4.5-3.3 nM. The above-mentioned affinities are preferably as measured using steady state cell affinity measurements, wherein cells are incubated at 4° C. using radioactively labeled antibody, where after cell-bound radioactivity is measured, as described in the Examples of WO 2015/130173.

Preferably, the bispecific antibodies used in the disclosed methods do not significantly affect the survival of cardiomyocytes. Cardiotoxicity is a known risk factor in ERBB2 targeting therapies and the frequency of complications is increased when trastuzumab is used in conjunction with anthracyclines thereby inducing cardiac stress.

The bispecific antibodies disclosed herein are preferably used in humans. thus, preferred antibodies are human or humanized antibodies. Tolerance of a human to a polypeptide is governed by many different aspects. Immunity, be it T-cell mediated, B-cell mediated or other is one of the variables that are encompassed in tolerance of the human for a polypeptide. The constant region of a bispecific antibody is preferably a human constant region. The constant region may contain one or more, preferably not more than 10, preferably not more than 5 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a human variable region when it has an amino acid sequence that is identical to an amino acid sequence of the variable region of a naturally occurring human antibody, but for the CDR region. The variable region of an ERBB2 binding VH, an ERBB3 binding VH, or a light chain in an antibody may contain one or more, preferably not more than 10, preferably not more than 5 amino-acid differences with the variable region of a naturally occurring human antibody, not counting possible differences in the amino acid sequence of the CDR regions. Such mutations occur also in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, preferably done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Such deimmunized murine heavy chain variable regions are less immunogenic in humans than the original murine heavy chain variable region. Preferably a variable region or domain is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

A bispecific antibody disclosed herein preferably comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. Preferably the constant region comprises an IgG constant region, more preferably an IgG1 constant region, preferably a mutated IgG1 constant region. Some variation in the constant region of IgG1 occurs in nature, such as for instance the allotypes G1m1, 17 and G1m3, and/or is allowed without changing the immunological properties of the resulting antibody. Typically between about 1-10 amino acid insertions, deletions, substitutions or a combination thereof are allowed in the constant region.

Preferred bispecific antibodies as disclosed herein comprise:
at least the CDR3 sequence, preferably at least the CDR1, CDR2 and CDR3 sequences, or at least the heavy chain variable region sequence, of an ERBB2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences; and/or
at least the CDR3 sequence, preferably at least the CDR1, CDR2 and CDR3 sequences, or at least the heavy chain variable region sequence, of an ERBB 3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or a heavy chain variable region sequence that differs in at most 15 amino acids, preferably in at most 1, 2, 3, 4, 5, 6, 7, 8, 9 or 10 amino acids, more preferably in at most 1, 2, 3, 4 or 5 amino acids, from the recited heavy chain variable region sequences.

CDR sequences are for instance varied for optimization purposes, preferably in order to improve binding efficacy or the stability of the antibody. Optimization is for instance performed by mutagenesis procedures where after the stability and/or binding affinity of the resulting antibodies are preferably tested and an improved ERBB2 or ERBB3-specific CDR sequence is preferably selected. A skilled person is well capable of generating antibody variants comprising at least one altered CDR sequence. For instance, conservative amino acid substitution is applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

Preferred antibodies comprise a variable domain that binds ERBB2, wherein the VH chain of said variable domain comprises the amino acid sequence of VH chain MF2926; MF2930; MF1849; MF2973; MF3004; MF3958 (is humanized MF2971); MF2971; MF3025; MF2916; MF3991 (is humanized MF3004); MF3031; MF2889; MF2913; MF1847; MF3001, MF3003 or MF1898; or comprises the amino acid sequence of VH chain MF2926; MF2930; MF1849; MF2973; MF3004; MF3958 (is humanized MF2971); MF2971; MF3025; MF2916; MF3991 (is humanized MF3004); MF3031; MF2889; MF2913; MF1847; MF3001, MF3003 or MF1898 as having at most 15, preferably 1, 2, 3, 4, 5, 6, 7, 8, 9 or 10 more preferably at most 1, 2, 3, 4 or 5, amino acid insertions, deletions, substitutions or a combination thereof with respect to the above mentioned VH chain sequence. The VH chain of the variable domain that binds ERBB2 preferably comprises the amino acid sequence of:
MF1849; or
MF2971 or a humanized version thereof, wherein said humanized version preferably comprises the amino acid sequence of MF3958; or
MF3004 or a humanized version thereof, wherein said humanized version preferably comprises the amino acid sequence of MF3991. In one embodiment, the VH chain of the variable domain that binds ERBB2 comprises the amino acid sequence of VH chain MF1849; or MF2971 or a humanized version thereof, wherein said humanized version preferably comprises the amino acid sequence of MF3958; or MF3004 or a humanized version thereof, wherein said humanized version preferably comprises the amino acid sequence of MF3991, wherein the recited VH sequences have at most 15, preferably 1, 2, 3, 4, 5, 6, 7, 8, 9 or 10, more preferably at most 1, 2, 3, 4 or 5, amino acid insertions, deletions, substitutions or a combination thereof with respect to the respective sequence. In a preferred embodiment the VH chain of the variable domain that binds ERBB2 comprises the amino acid sequence of MF3958; or comprises the amino acid sequence of MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence.

The VH chain of the variable domain that binds ERBB3 preferably comprises the amino acid sequence of VH chain MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074; or comprises the amino acid sequence of VH chain MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence. The VH chain of the variable domain that binds ERBB3 preferably comprises the amino acid sequence of MF3178, MF3176, MF3163, MF6058, MF6061 or MF6065; or comprises the amino acid sequence of MF3178, MF3176, MF3163, MF6058, MF6061 or MF6065 having at most 15, preferably 1, 2, 3, 4, 5, 6, 7, 8, 9 or 10, more preferably in at most 1, 2, 3, 4 or 5, amino acid insertions, deletions, substitutions or a combination thereof with respect to the respective VH chain sequence. In a preferred embodiment the VH chain of the variable domain that binds ERBB3 comprises the amino acid sequence of MF3178; or comprises the amino acid sequence of MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect to the VH chain sequence. Preferably, the above-mentioned amino acid insertions, deletions and substitutions are not present in the CDR3 region. The above-mentioned amino acid insertions, deletions and substitutions are also preferably not present in the CDR1 and CDR2 regions. The above-mentioned amino acid insertions, deletions and substitutions are also preferably not present in the FR4 region.

Preferably, the antibody comprises at least the CDR1, CDR2 and CDR3 sequences of MF1849, MF2971, MF3958, MF3004 or MF3991, most preferably at least the CDR1, CDR2 and CDR3 sequences of MF3958. Said antibody preferably comprises at least the CDR1, CDR2 and CDR3 sequences of MF3178, MF3176, MF3163, MF6058, MF6061 or MF6065, most preferably at least the CDR1, CDR2 and CDR3 sequence of MF3178.

Preferably, the ERBB2 specific heavy chain variable region comprises the amino acid sequence of the VH chain MF3958 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). They are also preferably not present in the FR4 region. An amino acid substitution is preferably a conservative amino acid substitution.

Preferably, the ERBB3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

Preferably, the ERBB2 specific heavy chain variable region comprises the amino acid sequence of the VH chain MF3991 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH (preferably wherein said insertions, deletions, substitutions are not in CDR1, CDR2, or CDR3). They are also preferably not present in the FR4 region. An amino acid substitution is preferably a conservative amino acid substitution.

Preferably, the ERBB3 specific heavy chain variable region comprises the amino acid sequence of the VH chain MF3178 having at most 15, preferably 1, 2, 3, 4, 5, 6, 7, 8, 9 or 10, more preferably at most 1, 2, 3, 4 or 5, amino acid insertions, deletions, substitutions or a combination thereof with respect said VH. The one or more amino acid insertions, deletions, substitutions or a combination thereof are preferably not in the CDR1, CDR2 and CDR3 region of the VH chain. They are also preferably not present in the FR4 region. An amino acid substitution is preferably a conservative amino acid substitution.

Preferably, the first antigen-binding site of the antibody comprises at least the CDR1, CDR2 and CDR3 sequences of MF3958, or CDR1, CDR2 and CDR3 sequences that differ in at most three, preferably in at most two, preferably in at most one amino acid from the CDR1, CDR2 and CDR3 sequences of MF3958, and wherein said second antigen-binding site comprises at least the CDR1, CDR2 and CDR3 sequence of MF3178, or CDR1, CDR2 and CDR3 sequences that differ in at most three, preferably in at most two, preferably in at most one amino acid from the CDR1, CDR2 and CDR3 sequences of MF3178.

Preferably, the bispecific antibody comprises i) a first antigen binding site comprising an ERBB2 specific heavy chain variable region comprising the CDR1, CDR2, and CDR3 sequence of MF3958 and a light chain variable region and ii) a second antigen binding site comprising an ERBB3 specific heavy chain variable region comprising the CDR1, CDR2, and CDR3 sequence of MF3178 and a light chain variable region.

Preferably, the ERBB2 specific heavy chain variable region has the MF3958 sequence and the ERBB3 specific heavy chain variable region has the MF3178 sequence. This combination is also referred to as the PB4188 antibody. Preferably, the PB4188 antibody is afucosylated.

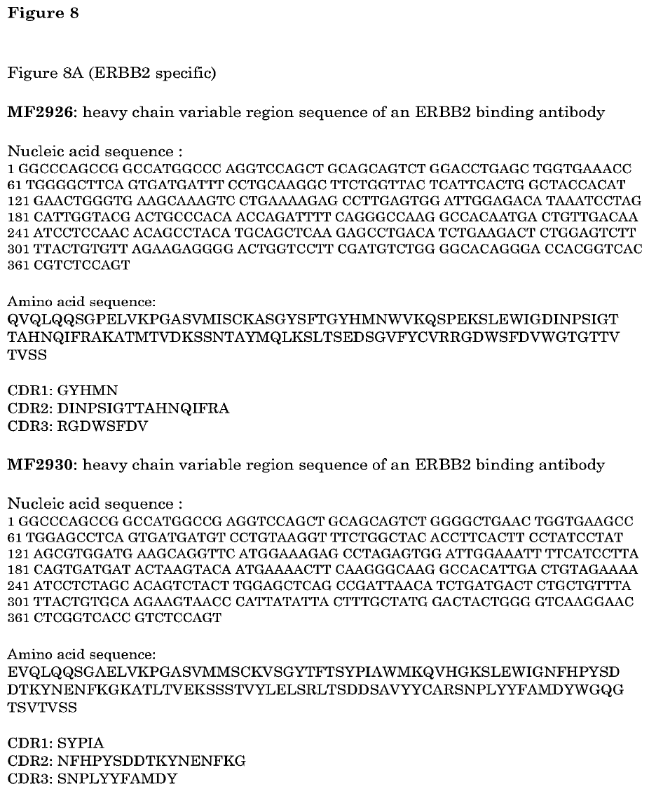

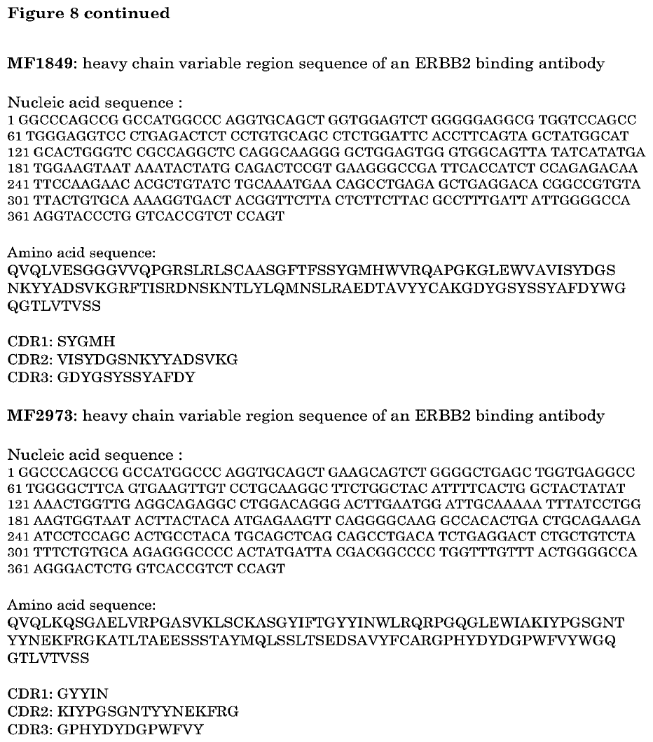

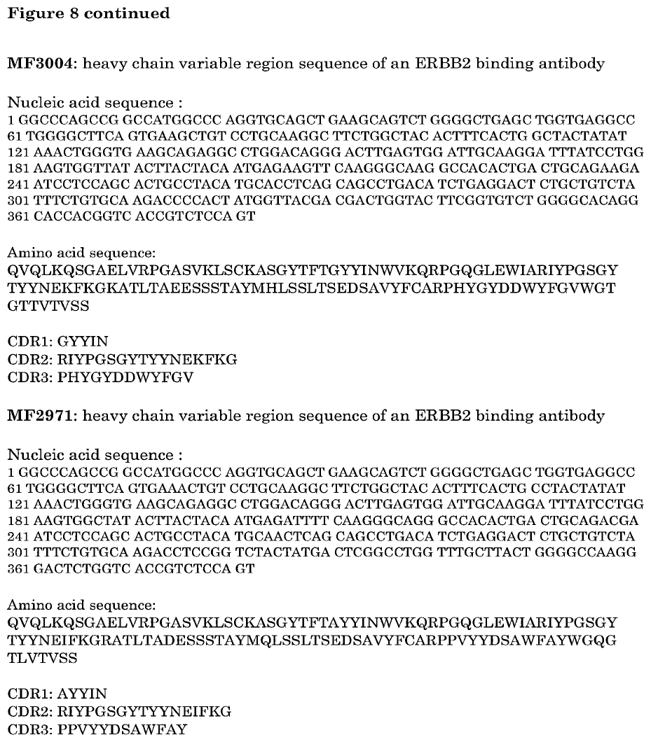

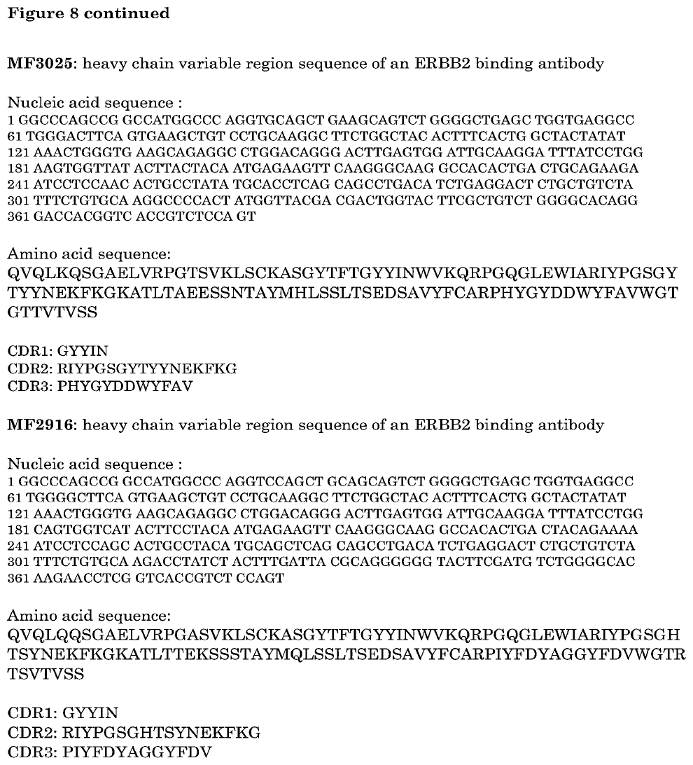

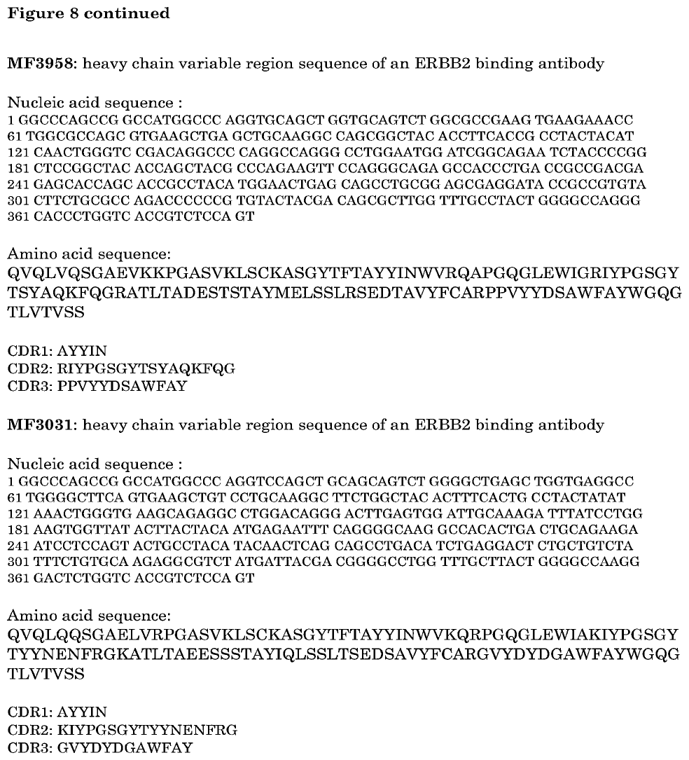

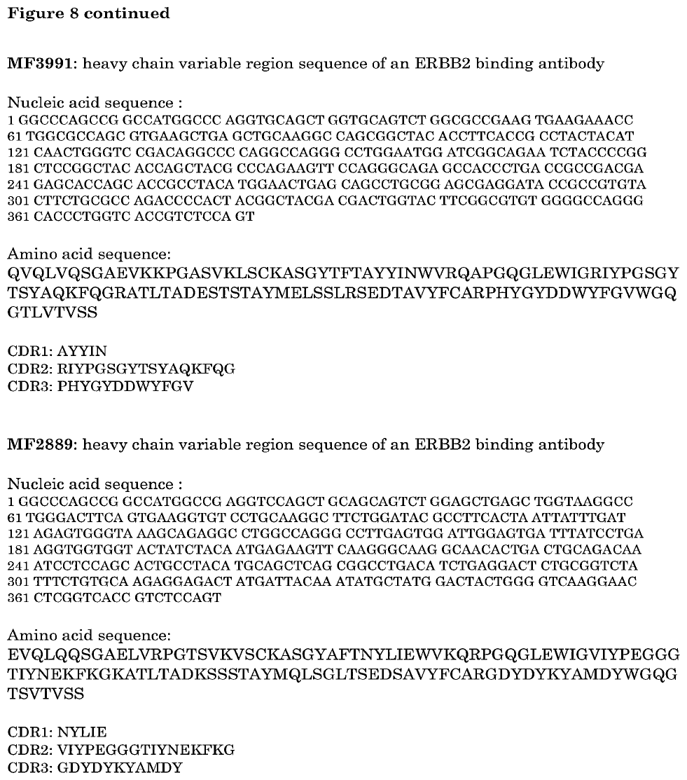

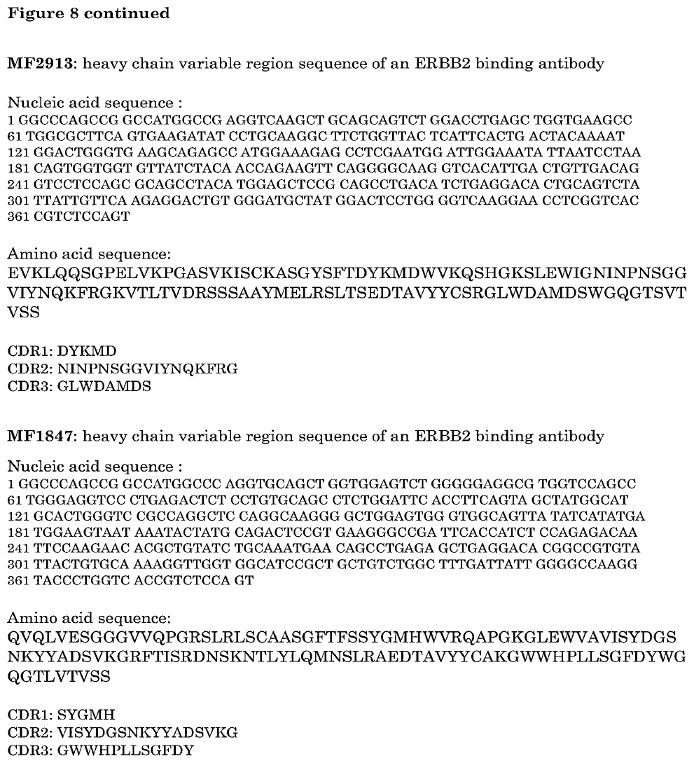

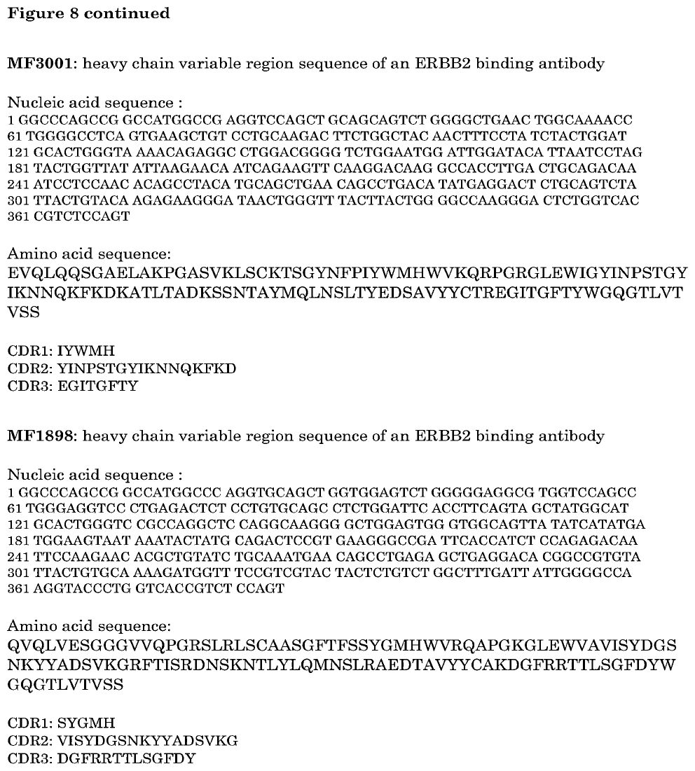

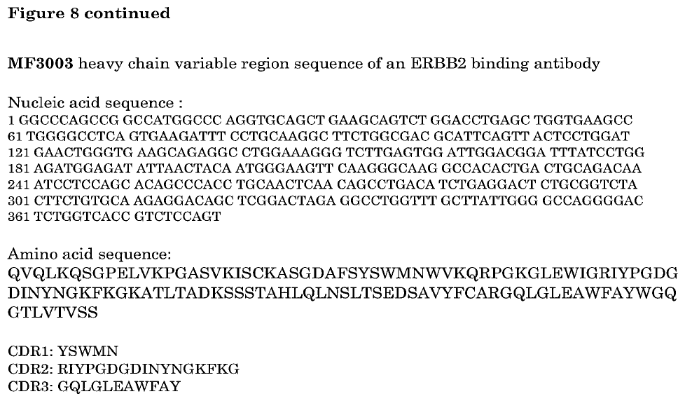

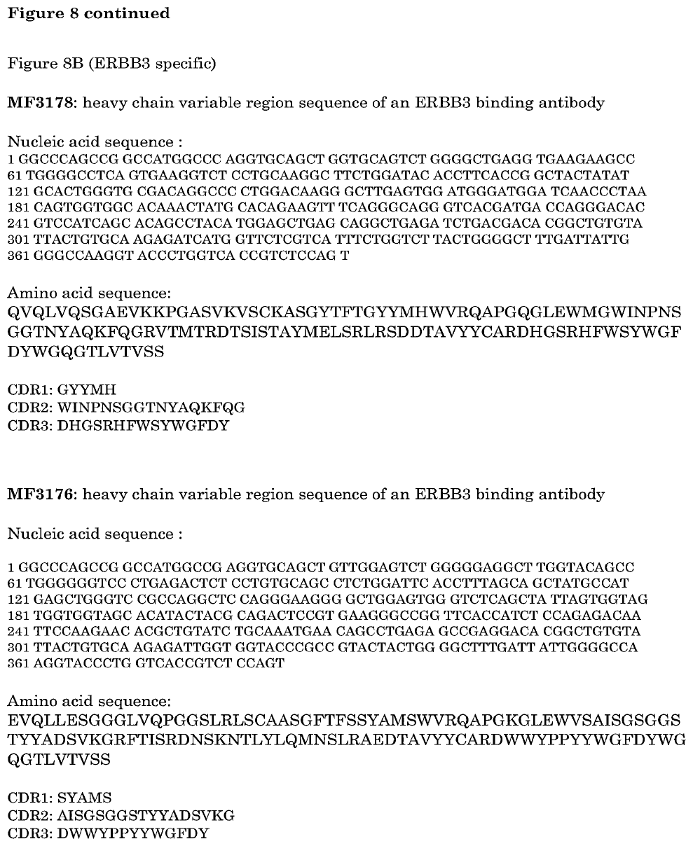

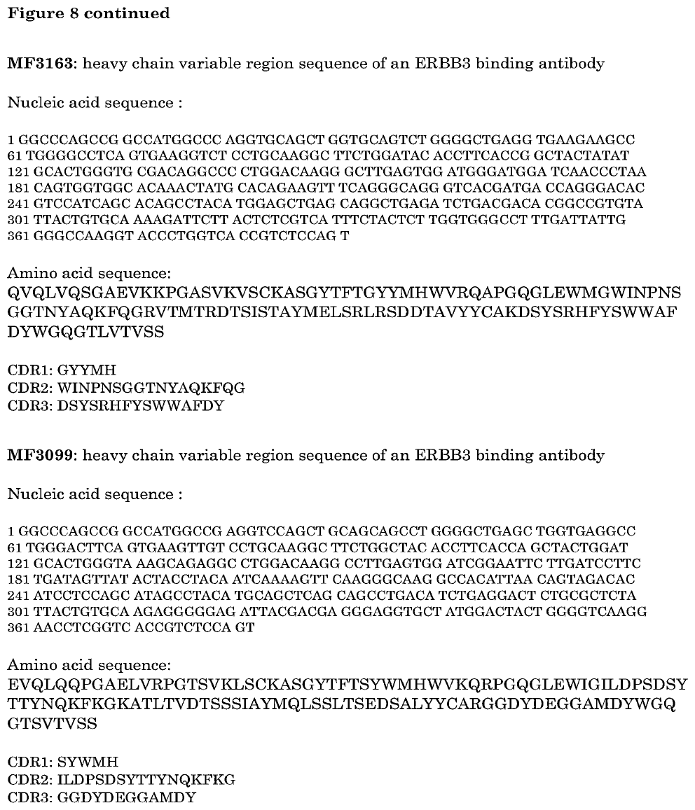

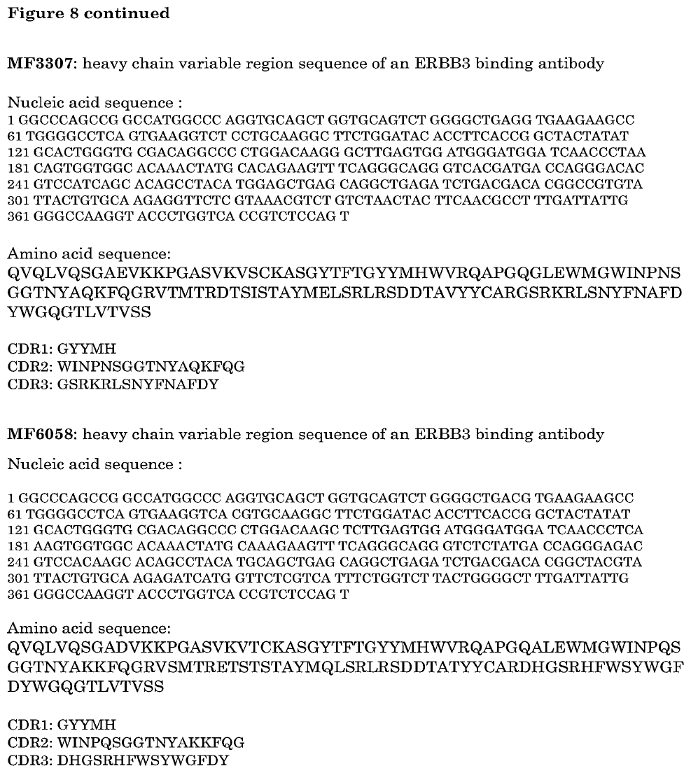

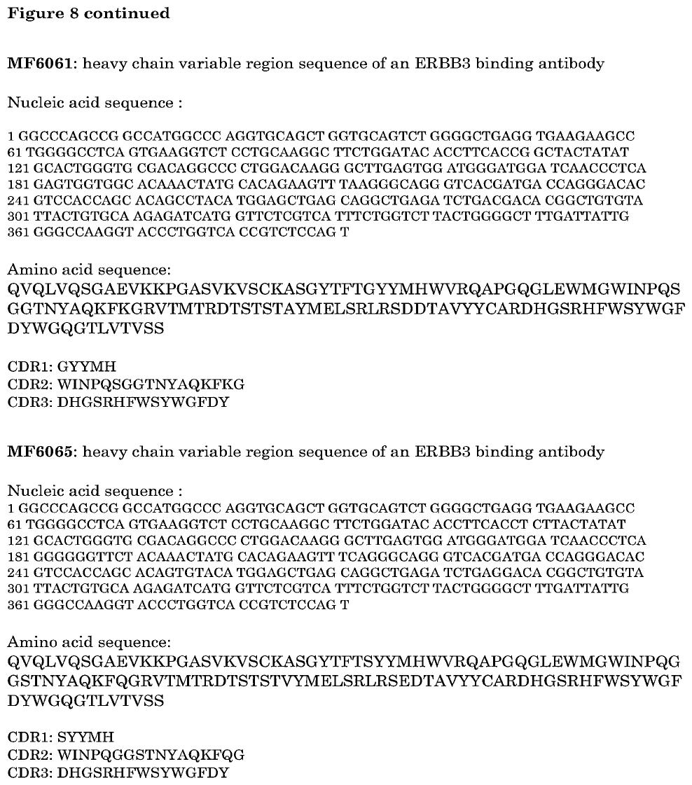

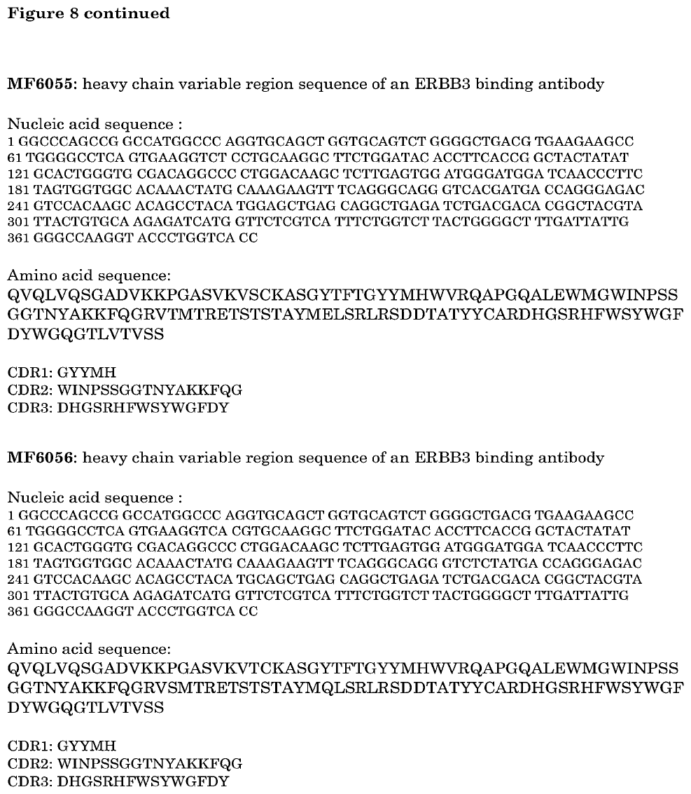

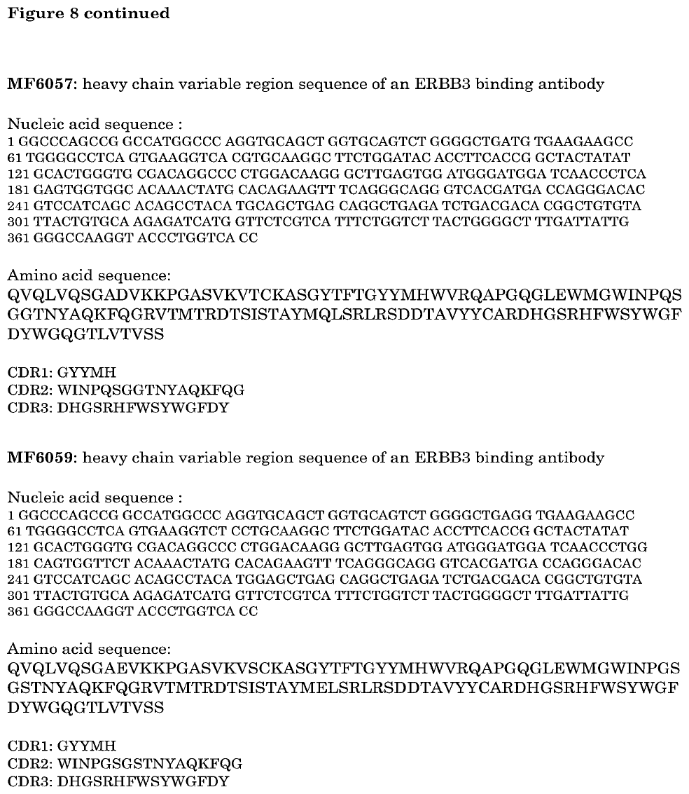

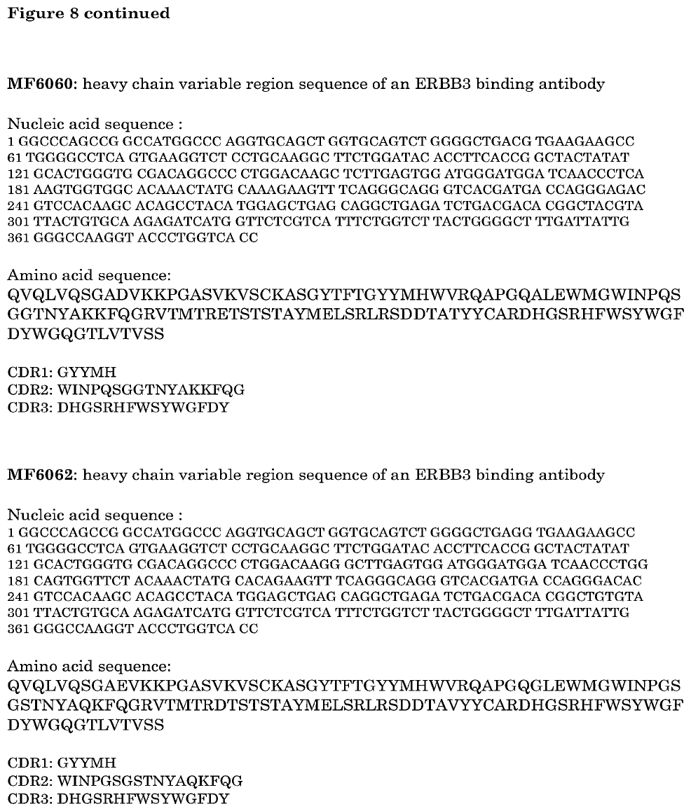

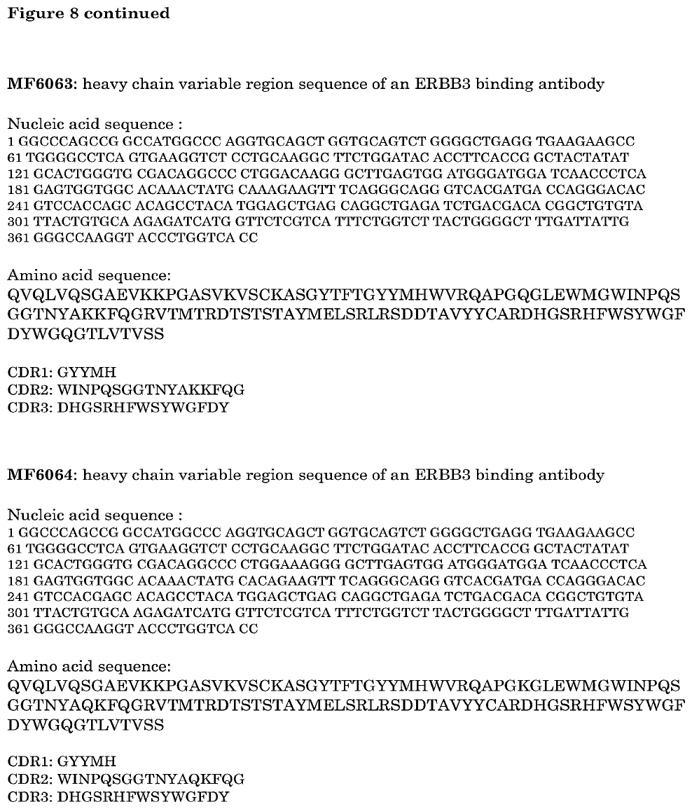

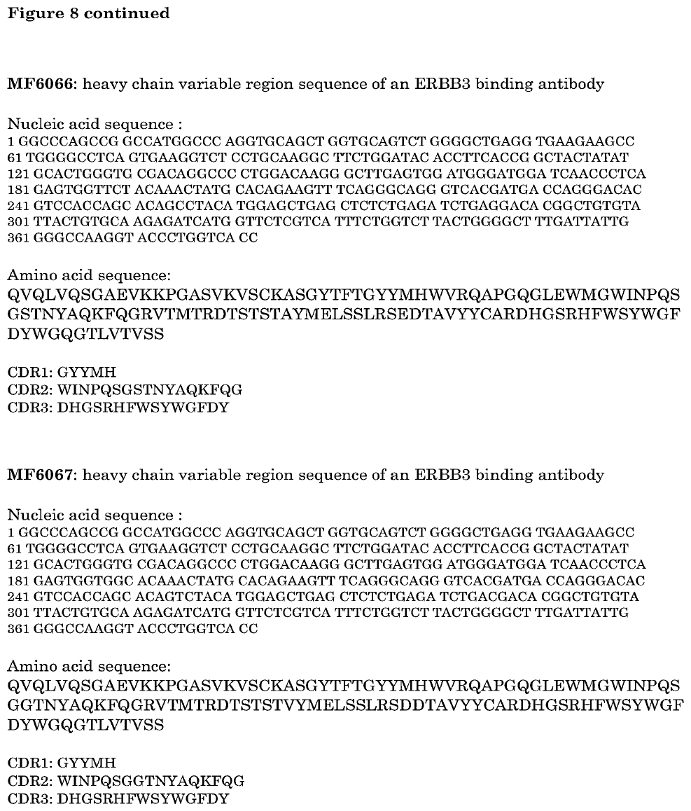

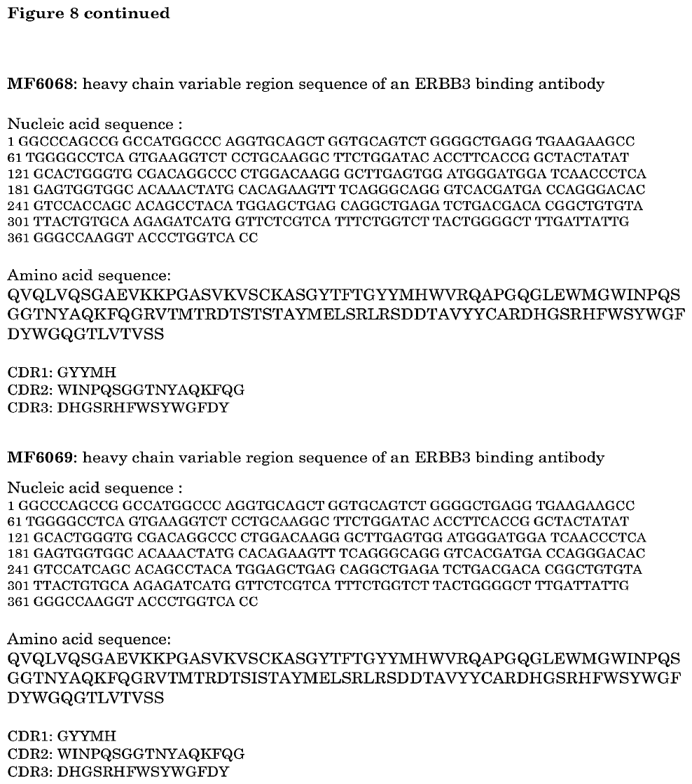

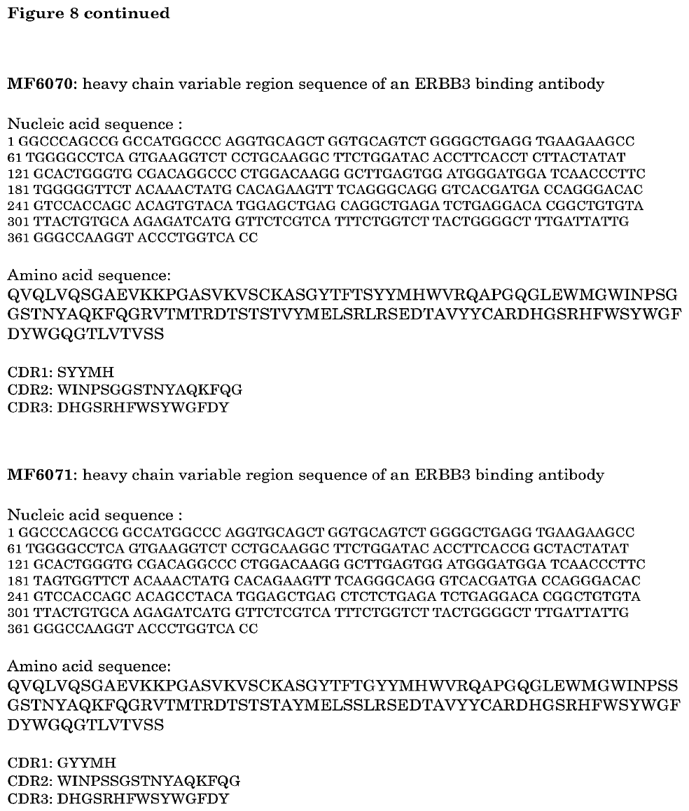

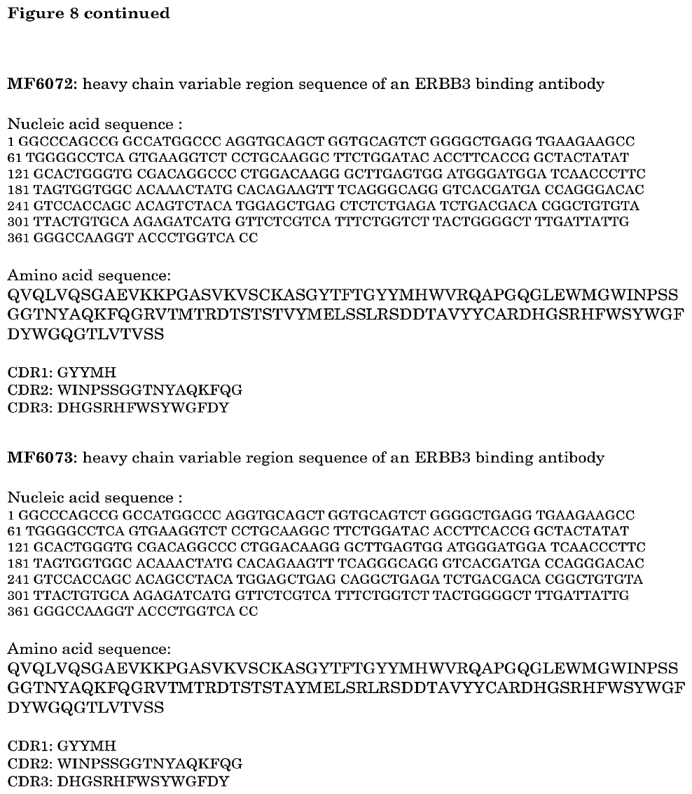

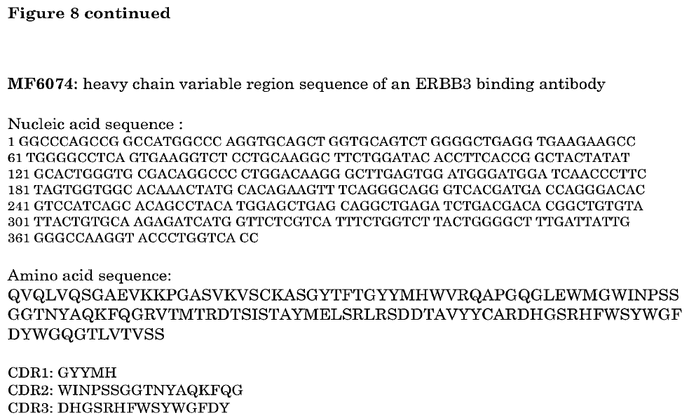

Preferably, the bispecific antibody comprises the “heavy chain for ERBB2 binding” as depicted in FIG. 8a and the “heavy chain for ERBB3 binding” as depicted in FIG. 8b.

Preferably, the antigen binding sites of the bispecific antibody comprise a common light chain as defined herein, preferably a germline common light chain, preferably the rearranged germline human kappa light chain IgVκ1-39*01/IGJκ1*01 or a fragment or a functional derivative thereof (nomenclature according to the IMGT database worldwide web at imgt.org). The terms rearranged germline human kappa light chain IgVκ1-39*01/IGJκ1*01, IGKV1-39/IGKJ1, huVκ1-39 light chain or in short huVκ1-39 are used. The light chain can have 1, 2, 3, 4 or 5 amino acid insertions, deletions, substitutions or a combination thereof. The mentioned 1, 2, 3, 4 or 5 amino acid substitutions are preferably conservative amino acid substitutions, the insertions, deletions, substitutions or a combination thereof are preferably not in the CDR3 region of the VL chain, preferably not in the CDR1, CDR2 or CDR3 region or FR4 region of the VL chain. Preferably, the first antigen binding site and the second antigen binding site comprise the same light chain variable region, or rather, a common light chain. Preferably, the light chain variable region comprises a CDR1 having the sequence (RASQSISSYLN) (SEQ ID NO: 1), a CDR2 having the sequence (AASSLQS) (SEQ ID NO: 2), and a CDR3 having the sequence (QQSYSTPPT) (SEQ ID NO: 3). Preferably, the light chain variable region comprises the common light chain sequence depicted FIG. 6.

Various methods are available to produce bispecific antibodies and are discussed in WO 2015/130173. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection.

The ratio of bispecific to other antibodies that are produced by the cell can be increased in various ways. Preferably, the ratio is increased by expressing not two different light chains but two essentially identical light chains in the cell. This concept is in the art also referred to as the “common light chain” method. When the essentially identically light chains work together with the two different heavy chains allowing the formation of variable domains with different antigen-binding sites and concomitant different binding properties, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. The art describes various ways in which such heterodimerization of heavy chains can be achieved. A preferred method is described in PCT application No. PCT/NL2013/050294 (WO 2013/157954 A1), which are incorporated herein by reference. Methods and means are disclosed for producing bispecific antibodies from a single cell, whereby means are provided that favor the formation of bispecific antibodies over the formation of monospecific antibodies.

For the purpose of clarity and a concise description features are described herein as part of the same or separate embodiments, however, it will be appreciated that the scope of the invention may include embodiments having combinations of all or some of the features described.

### Clauses

1. A bispecific antibody that comprises an antigen binding site that can bind an extracellular part of ERBB2 and an antigen binding site that can bind an extracellular part of ERBB3 for use in a method of treatment of a cancer harboring a ERBB3 mutation in a subject in need thereof.

2. A method of treatment of a subject having a cancer harboring a ERBB3 mutation, the method comprising administering a bispecific antibody that comprises an antigen binding site that can bind an extracellular part of ERBB2 and an antigen binding site that can bind an extracellular part of ERBB3.

3. The method according to any one of the preceding clauses, wherein the bispecific antibody comprises a first antigen-binding site that binds domain I of ERBB2 and a second antigen-binding site that binds domain III of ERBB3.

4. The method according to any one of the preceding clauses, wherein the bispecific antibody comprises
i) at least the CDR1, CDR2 and CDR3 sequences of an ERBB2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898 or wherein said antibody comprises CDR sequences that differ in at most 3 amino acids, preferably in at most 2 amino acids, preferably in at most 1 amino acid from the CDR1, CDR2 and CDR3 sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) at least the CDR1, CDR2 and CDR3 sequences of an ERBB3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

5. The method according to any one of the preceding clauses, wherein the bispecific antibody comprises
i) an ERBB2 specific heavy chain variable region sequence selected from the group consisting of the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 and MF1898, or wherein said antibody comprises a heavy chain variable region sequence that differs in at most 15 amino acids from the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) an ERBB3 specific heavy chain variable region sequence selected from the group consisting of the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

6. The method according to any one of the preceding clauses, wherein the bispecific antibody comprises heavy chain variable regions MF3958 and MF3178.

7. The method according to any one of the preceding clauses, wherein the bispecific antibody comprises a variable domain that comprises said first antigen binding site and the variable domain that comprises said second antigen binding site of said bispecific antibody preferably comprise a light chain variable region comprising a CDR1 having the sequence (RASQSISSYLN) (SEQ ID NO: 1), a CDR2 having the sequence (AASSLQS) (SEQ ID NO: 2), and a CDR3 having the sequence (QQSYSTPPT) (SEQ ID NO: 3), according to KABAT numbering or according to the IMGT numbering system, the CDRs of said light chain variable region are QSISSY (SEQ ID NO: 5), AAS and QQSYSTPPT (SEQ ID NO: 6), respectively.

8. The method according to any one of the preceding clauses, wherein the cancer harboring a ERBB3 mutation is colorectal cancer, gastric cancer, non-small-cell lung cancer (NSCLC) adenocarcinoma (adeno), NSCLC squamous cell carcinoma, renal carcinoma, melanoma, ovarian cancer, lung large cell carcinoma, esophageal cancer, small-cell lung cancer, hepatocellular cancer (HCC), breast cancer, hormone-positive breast cancer, glioblastoma, and head and neck cancer, gastric adenocarcinoma or an esophago-gastric cancer, a pancreatic cancer, a pancreatic ductal adenocarcinoma, a sarcoma, a bladder cancer, a gallbladder cancer, head and neck cancer, a prostate cancer, uterine/endometrial cancer, a lung cancer such as non-small cell lung cancer, including invasive mucinous adenocarcinoma.

9. The method according to any one of the preceding clauses, wherein the ERBB3 mutation comprises a mutation in domain I, II, III or IV of ERBB3.

10. The method according to any one of the preceding clauses, wherein the ERBB3 mutation comprises a mutation in the intracellular domain, preferably in the tyrosine kinase domain, preferably involving an amino acid residue in the range of 710-964, preferably at positions Q809, 5846, Q865 or E928.

11. The method according to any one of the preceding clauses, wherein the ERBB3 mutation comprises a mutation which causes ligand independent heterodimerization of ERBB2 and ERBB3.

12. The method of claim 11, wherein the mutation promotes the PI3K pathway.

13. The method according to any one of the preceding clauses, wherein the ERBB3 mutation comprises a mutation in the extracellular domain of ERBB3.

14. The method according to any one of the preceding clauses, wherein the ERBB3 mutation comprises a mutation in the intracellular domain of ERBB3.

15. The method according to any one of the preceding claims, wherein a mutation in ERBB3 is a mutation over the non-mutated sequence according to SEQ ID No: 1.

16. The method according to any one of the preceding clauses, wherein the ERBB3 mutation comprises one or more of the following mutations
M60N, where N is any naturally occurring amino acid, preferably K;
M91N, where N is any naturally occurring amino acid, preferably I;
R103N, where N is any naturally occurring amino acid, preferably G;
V104N, where N is any naturally occurring amino acid, preferably L or M;
R135N, where N is any naturally occurring amino acid, preferably C;
F219N, where N is any naturally occurring amino acid, preferably L;
H228N, where N is any naturally occurring amino acid, preferably Q;
A232N, where N is any naturally occurring amino acid, preferably V;
P262N, where N is any naturally occurring amino acid, preferably H or L or S;
G284N, where N is any naturally occurring amino acid, preferably R;
D297N, where N is any naturally occurring amino acid, preferably Y or A or H or N or V;
K329N, where N is any naturally occurring amino acid, preferably E or I or T;
E332N, where N is any naturally occurring amino acid, preferably K;
T355N, where N is any naturally occurring amino acid, preferably A or I or P;
R475N, where N is any naturally occurring amino acid, preferably W;
Q809N, where N is any naturally occurring amino acid, preferably R;
S846N, where N is any naturally occurring amino acid, preferably I;
Q865N, where N is any naturally occurring amino acid, preferably H;
E928N, where N is any naturally occurring amino acid, preferably G.

17. The method according to any one of the preceding clauses, wherein the ERBB3 does not have a mutation at R426.

18. The method according to any one of the preceding clauses, wherein the cancer lacks detectable mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, and PTEN.

19. The method according to any one of the preceding clauses, wherein the cancer lacks detectable amplifications in one or more genes selected from the group including c-MET, c-MYC, EGFR, ERBB2, and MDM2 amplification.

20. The method according to any one of the preceding clauses, wherein the cancer does not have a detectable PTEN loss.

21. The method according to any one of the preceding clauses, wherein the administration of the bispecific antibody comprises a first line therapy for treatment of a cancer harboring a ERBB3 mutation in said subject.

22. The method according to clauses 1 to 20, wherein the cancer has progressed after the subject having received a prior treatment.

23. The method of clause 22, wherein the prior treatment comprises chemotherapy, checkpoint inhibitor therapy (including anti-PD1 and anti-PD-L1 approved therapies and applicable therapies in clinical development), anti-ERBB2 or anti-ERBB3 or anti-VEGFR2 (vascular endothelial growth factor receptor 2), or a prior treatment with a tyrosine kinase inhibitor (TKI) of ERBB2 or of VEGFR2-TIE2, or with a combination of TKIs or any of the above.

24. The method of clause 23, wherein the chemotherapy comprises gemcitabine, capecitabine, carboplatin, a taxane, such as docetaxel or paclitaxel, 5-fluorouracil (with or without radiotherapy), vinorelbine, carmustine, doxorubicin, epirubicin, mitoxantrone, vinblastine, cisplatin (or pemetrexed), oxaliplatin, carboplatin, ifosfamide, mytomycin C, vindesine, etoposide, Folfox (i.e. a combination of 5-fluorouracil, leucovorin, and oxaliplatin) or Folfiri (i.e. a combination of leucovorin, 5-fluorouracil and irinotecan), Folfirinox (a combination of leucovorin, 5-fluorouracil, irinotecan and oxaliplatin) or any combination thereof.

25. The method of clause 23, wherein the he checkpoint inhibitor therapy according to the present invention includes an anti-PD1, anti-PD-L1, and anti-CTLA4 therapeutic moiety and preferably comprises ipilimumab, nivolumab, pembrolizumab, atezolizumab, avelumab, durvalumab or cemiplimab, or any combination thereof.

26. The method of clause 23, wherein the TKI is lapatinib, canertinib, neratinib, tucatinib (irbinitinib), CP-724714, tarloxitinib, mubritinib, afatinib, varlitinib, and dacomitinib, afatinib, or any combination thereof.

27. The method of clause 23, wherein the VEGFR2 targeted treatment is ramucirumab or regorafenib.

28. The method according to any one of the preceding clauses, wherein the cancer comprises mutation A232V in the ERBB3 protein.

29. The method according to any one of the preceding clauses, wherein the cancer is bladder cancer comprising mutation A232V in the ERBB3 protein.

30. The method according to any one of the preceding clauses, wherein the cancer comprises mutation V104M in the ERBB3 protein.

31. The method according to any one of the preceding clauses, wherein the cancer is ovarian cancer, such as clear cell carcinoma, comprising mutation V104M in the ERBB3 protein.

### EXAMPLES

As used herein “MFXXXX” wherein X is independently a numeral 0-9, refers to a Fab comprising a variable domain wherein the VH has the amino acid sequence identified by the 4 digits depicted in FIG. 8. Unless otherwise indicated the light chain variable region typically has a sequence of FIG. 6b. The light chain constant region in the examples has a sequence as depicted in FIG. 6c. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIG. 7d and FIG. 7e. Bispecific antibodies in the examples have an Fc tail with a KK/DE CH3 heterodimerization domain, a CH2 domain and a CH1 domain as indicated in FIG. 7, a common light chain as indicated in FIG. 6a and a VHs as specified by the MF numbers.

### Example 1

The capacity of ERBB3 mutations to induce survival of ERBB2-expressing BAF3 cells and the ability of ERBB2×ERBB3 bispecific antibodies, including MF3958×MF3178 to revert the mutant ERBB3-induced survival of ERBB2-BAF3 cells is evaluated and performed essentially as described in Jaiswal et al. Vol. 23, Issue 5, Cancer Cell (2013).

ERBB2: ERBB3 BAF3 and 19 point mutation cell lines are generated, with Heregulin (HRG) titration performed on BAF3-ERBB2 (WT)/ERBB3 (WT).

ERBB2×ERBB3 bispecific antibody titration, including with MF3958×MF3178 is performed on BAF3-ERBB2 (WT)/ERBB3 (WT) in the presence of a concentration of HRG that results in maximal survival stimulation (Effective Concentration, EC100).

Model systems are treated with ERBB2×ERBB3 bispecific antibodies, including MF3958×MF3178 (EC100 as determined in the previous experiment) with the WT and mutation cell lines.

The project consists of the following:

1. Heregulin titration curve for BAF3 cells expressing both ERBB2-wt and ERBB3-wt Heregulin titration start from 0.3 ng/ml, with 9 concentrations in total.

2. ERBB2×ERBB3 bispecific antibodies, including MF3958×MF3178 titration curves for BAF3 cells expressing both ERBB2-wt and ERBB3-wt in the presence of Heregulin at EC100 as determined in Step 1.

For MF3958×MF3178, starting concentration from 0.03 ng/ml is used, with 9 concentrations in total on BAF3-ERBB2 (WT)/ERBB3 (WT).

3. ERBB3-mutant constructs are generated and express ERBB3-mutant constructs in ERBB2-expressing BAF3. The expression level of both ERBB2 and ERBB3 in all cell lines are validated by FACS.

The following ERBB3 mutational cell lines are generated:
A232V
D297Y
E332K
E928G
F219L
G284R
H228Q
K329E
M60K
M91I
Q809R
Q865H
R103G
R135C
R475W
S846I
T355I
V104L
V104M

4. Model systems are treated with a ERBB2×ERBB3 bispecific antibody, including MF3958×MF3178 (EC100). All model systems generated are treated with MF3958×MF3178 (EC100) as determined in step 2 and with PBS (vehicle control). Treatment with the bispecific antibody will show inhibition of cell growth in the transformed cell lines with the Her3 mutants as indicated.

### Example 2

A Clinical Study with the Bispecific Antibody MF3958×MF3178, a Full Length IgG1 Bispecific Antibody Targeting ERBB2 and ERBB3, in Patients with Solid Tumors Harboring ERBB3 Mutations

Study Duration:

For the dose escalation part of the study (Part 1) 28 patients were recruited. Part 2 of the study is the dose expansion phase. The total duration of Part 2 is approximately 25-32 months; however, the actual duration is influenced by several variables, e.g., overall subject recruitment rate.

Number of Patients:

Twenty-eight (28) patients were enrolled in Part 1. For Part 2, at least 20 evaluable patients, and up to approximately 40, may be enrolled in the group with documented ERBB3 mutations, including ERBB3 mutation comprising any one of the following:
M60N, where N is any naturally occurring amino acid, preferably K;
M91N, where N is any naturally occurring amino acid, preferably I;
R103N, where N is any naturally occurring amino acid, preferably G;
V104N, where N is any naturally occurring amino acid, preferably L or M;
R135N, where N is any naturally occurring amino acid, preferably C;
F219N, where N is any naturally occurring amino acid, preferably L;
H228N, where N is any naturally occurring amino acid, preferably Q;
A232N, where N is any naturally occurring amino acid, preferably V;
P262N, where N is any naturally occurring amino acid, preferably H or L or S
G284N, where N is any naturally occurring amino acid, preferably R;
D297N, where N is any naturally occurring amino acid, preferably Y or A or H or N or V;
K329N, where N is any naturally occurring amino acid, preferably E or I or T;
E332N, where N is any naturally occurring amino acid, preferably K;
T355N, where N is any naturally occurring amino acid, preferably A or I or P;
R475N, where N is any naturally occurring amino acid, preferably W;
Q809N, where N is any naturally occurring amino acid, preferably R;
S846N, where N is any naturally occurring amino acid, preferably I;
Q865N, where N is any naturally occurring amino acid, preferably H;
E928N, where N is any naturally occurring amino acid, preferably G.

Preferably, patients' cancer harbor the following ERBB3 mutations in the extracellular domain, G284R, R013G, V104M, R135C, A232, D297, T355 or in the intracellular domain, E928G, S846I, Q865H or Q809R.

Preferably, patients' cancer cells harboring the ERBB3 mutation lack the following oncogenic drivers of KRAS, NRAS, PIK3CA, BRAF or the cell shows absence of oncogenic mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, PTEN.

Preferably cancer cells harboring the ERBB3 mutation lack the following oncogenic amplifications c-MET amplification, c-MYC amplification, EGFR amplification, ERBB2 amplification, MDM2 amplification.

Preferably cancer cells harboring the ERBB3 mutation do not have PTEN loss.

Patients who do not complete at least two cycles of study treatment due to other reasons than disease progression, are not evaluable for efficacy and are replaced in the respective group.

This Example describes Part 2 of the study.

Study Objectives:

Part 1

| Objective | |
|---|---|
| Primary: | |
| Determination | Evaluation of adverse events (AEs) and dose limiting |
| of the MTD | toxicities (DLT). |
| and/or MRD of | |
| MF3958 × | |
| MF3178. | |
| Secondary: | |
| To characterize | Frequency and nature of AEs/serious adverse events |
| the safety and | (SAEs). |
| tolerability of | |
| MF3958 × | |
| MF3178. | |
| PK profile of | Assessment of PK variables, including total exposure, |
| MF3958 × | maximum concentration (Cₘₐₓ) clearance, volume of |
| MF3178. | distribution (V), volume of distribution at steady state |
| | (Vₛₛ), half-life (t_{1/2}), AUC₀₋ₜ (area under the |
| | concentration versus time curve from time zero to |
| | time t), AUC_{0-∞} (area under the concentration versus |
| | time curve), tₘₐₓ (time to reach maximum |
| | concentration). |
| Immunogenicity | Incidence and serum titers of anti-drug antibodies |
| of MF3958 × | against MF3958 × MF3178. |
| MF3178. | |
| Evaluation of | Anti-tumor activity and clinical benefit assessed by |
| anti-tumor | RECIST v1.1 determining objective overall response |
| response and | rate (ORR), duration of response (DOR), progression- |
| clinical benefit | free survival (PFS) and survival; CBR is defined as |
| rate (CBR). | the proportion of patients in whom a complete |
| | response (CR) or partial response (PR) or stable |
| | disease (SD) is observed (where SD duration is a |
| | minimum of 12 weeks). |
| Exploratory | |
| (includes | |
| optional | |
| assessments): | |
| Presence of | Assessment of relevant tumor biomarkers and markers |
| biomarkers and | of MF3958 × MF3178 activity in archival and/or fresh |
| pharmaco- | tumor biopsy material and blood. The following |
| dynamic (PD) | candidate biomarkers are assessed: |
| responses to | HER2, HER3, pHER2, pHER3 & heregulin; |
| MF3958 × | KRAS, NRAS, PIK3CA, BRAF mutation status; |
| MF3178. | circulating tumor deoxyribonucleic acid (DNA) and |
| | mutations in genes associated with HER2/HER3 |
| | signaling; |
| | phosphorylated molecules in the MAPK and AKT |
| | signaling pathway. |

Part 2

| Objective | |
|---|---|
| Primary (safety): | |
| To characterize | Frequency and nature of AEs. |
| the safety and | |
| tolerability of | |
| MF3958 × | |
| MF3178. | |
| Primary (efficacy): | |
| To explore the | Overall response rate (ORR), DOR, CBR (defined |
| relationships | as the proportion of patients in whom a CR or PR |
| between the anti- | is observed, or SD of a minimum duration of 12 |
| tumor activity | weeks) per RECIST 1.1 as per local investigator's |
| of MF3958 × | assessment. The relationship between anti-tumor |
| MF3178 and | activity and biomarkers including expression of |
| disease-related | HER2, HER3, and heregulin are explored, and |
| biomarkers | serum biomarkers such as CA-125 (ovarian, |
| | endometrial) and CA-19-9 (gastric) |
| Secondary: | |
| PK profile of | Assessment of PK variables, including total |
| MF3958 × MF3178. | exposure, Cₘₐₓ, V, Vₛₛ, t_{1/2}, AUC₀₋ₜ, AUC_{0-∞}, tₘₐₓ. |
| | Population PK analysis |
| Immunogenicity of | Incidence and serum titers of anti-drug antibodies |
| MF3958 × MF3178. | against MF3958 × MF3178. |
| Evaluation of PFS | |
| and overall survival, | |
| duration of response | |
| Exploratory | |
| (includes optional | |
| assessments): | |
| Assessment of | The following candidate biomarkers are assessed if |
| other relevant | sufficient sample is available: |
| tumor biomarkers | Tumor sample |
| and markers of | pHER2, pHER3, HER2:HER3 dimerization; |
| MF3958 × MF3178 | Heregulin and (depending on availability) mutations |
| activity in | in cancer genes including those associated with |
| preferably fresh | HER2 and HER3 |
| tumor sample/ | Phosphorylated molecules in the MAPK and AKT |
| biopsy material or | signaling pathway. |
| archival and blood. | ERBB3 mutations, including mutation(s) comprising |
| | the extracellular domain of the ERBB3 protein, or is |
| | one that is intracellular, which promotes and/or is |
| | correlated with ligand independent |
| | heterodimerization of ERBB2 and ERBB3, such that |
| | it promotes the PI3K pathway and any one of the |
| | following: Specifically, ERBB3 mutation comprises |
| | any one of the following: M60N, where N is any |
| | naturally occurring amino acid, preferably K; M91N, |
| | where N is any naturally occurring amino acid, |
| | preferably I; R103N, where N is any naturally |
| | occurring amino acid, preferably G; V104N, where |
| | N is any naturally occurring amino acid, preferably |
| | L or M; R135N, where N is any naturally occurring |
| | amino acid, preferably C; F219N, where N is any |
| | naturally occurring amino acid, preferably L; |
| | H228N, where N is any naturally occurring amino |
| | acid, preferably Q; A232N, where N is any naturally |
| | occurring amino acid, preferably V; P262N, where |
| | N is any naturally occurring amino acid, preferably |
| | H or L or S; G284N, where N is any naturally |
| | occurring amino acid, preferably R; D297N, where |
| | N is any naturally occurring amino acid, preferably |
| | Y or A or H or N or V; K329N, where N is any |
| | naturally occurring amino acid, preferably E or I |
| | or T; E332N, where N is any naturally occurring |
| | amino acid, preferably K; T355N, where N is any |
| | naturally occurring amino acid ,referably A or I |
| | por P; R475N, where N is any naturally occurring |
| | amino acid, preferably W; Q809N, where N is any |
| | naturally occurring amino acid, preferably R; |
| | S846N, where N is any naturally occurring amino |
| | acid, preferably I; Q865N, where N is any |
| | naturally occurring amino acid, preferably H; |
| | E928N, where N is any naturally occurring amino |
| | acid, preferably G. |
| | Blood |
| | Fcgamma receptor polymorphism |
| | Circulating tumor DNA and mutation analysis in |
| | cancer genes including those associated with |
| | HER2/HER3 signaling; |
| | Circulating tumor cells and HER2 status |

Study Design:

This is a Phase I/II, open-label, multi-center, multi-national, dose escalation, single group assignment study to assess the safety, tolerability, PK, PD, immunogenicity and anti-tumor activity of MF3958×MF3178.

The study is designed in 2 parts:

Part 1

Part 1 of the study includes the investigation of nine dose levels: 40 mg, 80 mg, 160 mg in cohorts of 1 patient and 240 mg, 360 mg, 480 mg, 600 mg, 750 mg, and 900 mg in cohorts of 3 patients. MF3958×MF3178 was initially given over approximately 60 minutes on Day 1 of a 3-week treatment cycle. During Part 1 the infusion duration was extended to 2 hours with the option of increasing it up to 4 hours to mitigate infusion-related reactions (IRRs).

No dose limiting toxicities (DLTs) were experienced at any of the dose levels. Three additional patients were dosed in each of the 600 mg and 750 mg cohorts in order to have sufficient PK information.

As an MTD was not reached at the dose level of 900 mg, the Data Review Committee (DRC) for MF3958×MF3178-CL01 decided to assign the dose level of 750 mg as the RP2D of the study, based on the cumulative safety, available PK data and PK simulations.

Part 2

Part 2 includes a further characterization of the safety and tolerability of the selected dose level of MF3958×MF3178, as well as assessment of CBR, defined as the proportion of patients with a CR, PR or durable SD (SD for at least 12 weeks in duration), in expansion groups of selected patient populations.

A weekly dose regimen with a 4-week cycle is evaluated in newly recruited patients consisting of a flat dose of 400 mg weekly for the first 2 cycles, with an 800 mg loading dose for the initial administration. From cycle 3, MF3958×MF3178 is given at a dose of 400 mg weekly for 3 weeks followed by 1 week off. Mandatory pre-medication is administered to mitigate IRRs. However, corticosteroids are only mandatory prior to the loading dose of Day 1 of Cycle 1 and should only be used for subsequent infusions as per the investigator's discretion to manage IRRs.

Alternatively, a dosing regimen is followed comprising a weekly cycle with a flat dose of 400 mg weekly commenced after an initial administration of 800 mg. From week 3, the bispecific antibody of the invention is given at a weekly dose of 400 mg for 3 weeks followed by 1 week without dosing of the bispecific antibody of the invention.

Alternatively, a dosing regimen is followed comprising a bi-weekly cycle with a flat dose of 750 mg weekly commenced after an initial administration of a 750 mg infusion over a four-hour period, followed by a bi-weekly two-hour infusion of 750 mg in a four-week cycle. A further alternative comprises a tri-weekly administration of a flat dose of 750 mg per subject.

Safety of the weekly schedule is reviewed during a run-in period after the first 5 patients treated have completed at least 2 treatment cycles. The DRC reviews all safety data with a focus on incidence of grade 3-4 toxicities, incidence and severity of IRRs, and compliance. If the DRC review concludes that toxicity is unacceptable, the Sponsor continues patient enrolment with the 3-week cycle dose regimen until a sufficient number of patients have been enrolled per cohort.

No within-patient dose escalation is permitted in Part 2.

Patient populations of interest to be assessed in Part 2 of the study are:
Any solid tumor with documented ERBB3 mutations.

At least 20 and up to approximately 40 patients may be enrolled in each Group (C-F) including a minimum of 10 patients per cohort treated with the weekly recommended dose. Previously closed cohorts may be reopened.

Duration of Treatment

Patients in both Part 1 and 2 of the study may remain on treatment until disease progression, death, unacceptable toxicity or discontinuation for any other reason.

Data Review Committee (DRC):

All dose escalation decisions in Part 1 were made by a DRC who convened to review all available safety data and PK data. The DRC participants included the Principal Investigators (or their representatives), the Sponsor's Medical Director, the study Medical Monitor, study Pharmacovigilance Physician, study Project Manager, study Statistician, and invited experts as required (such as clinical pharmacology expert).

In Part 2, the DRC reviews the data following completion of the safety run-in period for the weekly dose before expanding the weekly dose regimen in all subsequent patients.

Study Assessments:

The study consists of a molecular pre-screening assessment up to a 4-week (28-day) screening period, followed by sequential treatment cycles until treatment withdrawal or termination for any reason. The treatment cycle duration is 3 weeks (21 days) for patients treated at the initial recommended dose in Part 2, and 4 weeks (28 days) for patients treated with the bi-weekly recommended dose in Part 2. All patients should attend an End of Treatment visit within 1 week after treatment cessation and a Final Study Visit 30 days after end of treatment or discontinuation from study.

Patients who have not progressed or withdrawn consent on completion of the Final Study Visit are followed up every 3 months for up to 2 years (approximately) to check their disease progression and/or survival status until the commencement of their next anti-cancer treatment.

Where ongoing evaluation of safety data and available PK, PD and anti-tumor activity data during the trial suggest that alternative dosing frequencies should be evaluated, or that other patient populations should be evaluated in Part 2, these modifications are clarified in a protocol amendment prior to commencing these evaluations.

Molecular Pre-Screening and Screening:

Molecular pre-screening is performed in local laboratories qualified to perform molecular screening for applicable ERBB3 mutations, absence of oncogenic mutational drivers, amplifications or PTEN loss, or through centralized testing having such capabilities.

Patients have applicable ERBB3 mutations lacking another oncogenic driver, including, but not limited to KRAS, NRAS, PIK3CA, BRAF or the cell shows absence of oncogenic mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, PTEN, lacking the following oncogenic amplifications c-MET amplification, c-MYC amplification, EGFR amplification, ERBB2 amplification, MDM2 amplification and that do not have PTEN loss are selected for treatment with the bispecific antibody comprising MF3958×MF3178.

Safety Assessments

Concurrent illnesses are captured at baseline; AEs and concomitant therapies are monitored throughout study participation. Safety assessments include reviewing Eastern Cooperative Oncology Group (ECOG) performance status, physical examination (including height and weight), vital signs and electrocardiograms (ECG). A cardiac function test of the Left Ventricular Ejection Fraction (LVEF) is also be carried out at Screening, end of Cycle 4 (or Cycle 5 Day 1), End of Study Visit, and at any time during the study if clinically indicated. Laboratory evaluations include clinical chemistry, hematology, coagulation tests, urinalysis and pregnancy testing. Note that a cytokine panel analysis was performed up until 1 Aug. 2017.

On all MF3958×MF3178 administration days, the patients must remain at the clinic for at least 60 minutes from the time of the end of infusion (longer where there are PK samples required) for observation and repeat vital signs prior to discharge from the clinic. Further additional safety assessments should be performed as clinically indicated and, if needed, duration of stay in clinic should be increased based on Investigator's judgment.

Immunogenicity Assessment

Serum titers of anti-MF3958×MF3178 antibodies are measured on Day 1 at pre-dose for each of Cycles 1, 2, 3, 4 and then every fourth cycle thereafter (Cycle 8, 12, 16 etc), and at the End of Treatment Visit and the Final Study Visit with a window of −3 days prior to the MF3958×MF3178 administration.

Pharmacokinetics Assessment

Part 1 and Part 2 initial recommended dose schedule: In Cycle 1, blood samples are collected for PK analysis on Day 1 at pre-dose, at end of infusion (EOI), and at 1, 2, 4, 8, 24 hours post EOI, then on Day 4 (or Day 3), Day 8 and Day 15. In Cycles 2-4, only a pre-dose and EOI blood sample is collected.

Part 2 weekly recommended dose schedule: In Cycle 1, blood samples are collected for PK analysis on Day 1 at pre-dose, EOI, 2, 4, 24 hours post EOI, then predose on Days 8 and 15, and predose and EOI on Day 22. In Cycles 2 and 3, a pre-dose and EOI blood sample is collected on Day 15. In Cycle 4 blood samples are collected pre-dose on Day 1, and pre-dose and EOI on Day 15. Every 2 cycles thereafter (Cycles 6, 8, 10 etc) a pre-dose blood sample is collected on Day 15.

Tumor Assessment

Tumor assessment is evaluated according to RECIST version 1.1 per local investigator. Imaging is obtained at Screening and at the end of every 2 cycles of treatment for patients receiving the 3-week cycle regimen and every 8 weeks for patients receiving the bi-weekly dosing regimen.

Biomarker and Pharmacodynamics Assessments

A range of biomarker and pharmacodynamic tests are performed on archived and/or fresh tumor sample material and/or blood (liquid biopsy), depending on availability of archived or existing tumor tissue, consent for further tumor samples, and consent for specific biomarker testing.

The following candidate biomarkers are assessed in case sufficient sample is available:
HER2, HER3, HER2:HER3 dimerization, phosphorylated HER2 (pHER2) and HER3 (pHER3) and heregulin;
Circulating plasma tumor DNA (ctDNA) and tumor sample DNA (depending on availability) are used to examine mutations in cancer genes including those associated with HER2 and HER3 signaling
Phosphorylated molecules in the MAPK and AKT signaling pathway;
Fcgamma receptor polymorphism;
Circulating tumor cells for HER2;
ERBB3 mutations

No germ line DNA assessment is included (except for Fcgamma receptor polymorphism).

At baseline the patient is requested to provide a mandatory tumor sample tissue, preferably a block, which could be from fresh or archival tissue. The sponsor indicates the preference for fresh tissue. Archival is acceptable and should have been taken within 2 years from screening other than for NSCLC which must be within 1 year. In addition the patient is requested optionally to provide a tumor sample/biopsy at the end of Cycle 4 and optionally at the End of Treatment Visit.

Blood samples are also taken at these time points for the purpose of liquid biopsy testing.

Eligibility Criteria:

The study enrolls patients with a ERBB3 mutation positive cancer, lack of oncogenic mutations, amplifications and PTEN loss.

General Inclusion Criteria for Part 2
1. Age 18 years or older;
2. At least one measurable lesion according to RECIST v1.1 or evaluable disease for a limited number of patients in Group H;
3. Performance status of ECOG 0 or 1;
4. Estimated life expectancy of at least 12 weeks;
5. Toxicities incurred as a result of previous anti-cancer therapy resolved to ≤Grade 1 (as defined by NCI CTCAE v4.03), except for alopecia, Grade 2 sensory neurotoxicity, or any other toxicity that in the opinion of the investigator does not affect the assessment of adverse events related to the study drug;
6. Treatment with anti-cancer medication or investigational drugs within the following intervals before the first dose of MF3958×MF3178:
   a. >14 days or >5 half-lives prior to study entry, whichever is shorter.
   b. >14 days for radiotherapy. Note: A less than 1-week wash-out period is permitted only for palliative radiation to non-CNS disease with Sponsor app
7. Patient has recovered from prior surgery or other procedure or complication to ≤Grade 2 or to baseline condition that in opinion of the investigator does not affect the assessment of adverse events related to the study drug;
8. Laboratory values at Screening:
   a. Absolute neutrophil count ≥1.5×10⁹/L without colony stimulating factor support;
   b. Platelets ≥100×10⁹/L;
   c. Hemoglobin ≥8 g/dL or ≥2.2 mmol/L (not transfusion dependent);
   d. Alanine aminotransferase (ALT), aspartate aminotransferase (AST)≤3×upper limit of normal (ULN) and total bilirubin ≤1.5×ULN; in cases of metastatic liver involvement, ALT/AST ≤5×ULN and total bilirubin ≤2×ULN will be allowed; in cases of antecedents of Gilbert's syndrome when total bilirubin ≤3.0×ULN or direct bilirubin ≤1.5×ULN will be allowed;
   e. Estimated glomerular filtration rate (GFR) of >30 mL/min based on the CockroftGault formula;
9. Able to provide at baseline a mandatory tumor biopsy sample (FFPE), preferably a block. If safe/feasible, a fresh FFPE biopsy sample is preferred; archival tissue is acceptable (preferably not more than 2 years old); NOTE: For patients who received afatinib or other HER-targeting agents, a biopsy collected after the last line of treatment is strongly preferred to assess for mechanisms of acquired resistance.
10. Negative pregnancy test results available as defined by urine or blood human chorionic gonadotropin (hCG) test during Screening and within 7 days of Cycle 1, Day 1 in women of childbearing potential (defined as women ≤50 years of age or history of amenorrhea for ≤12 months prior to study entry);
11. Sexually active male and female patients of childbearing potential must agree to use an effective method of birth control (e.g., barrier methods with spermicides, oral or parenteral contraceptives and/or intrauterine devices) during the entire duration of the study and for 6 months after final administration of MF3958×MF3178. Note that sterility in female patients must be confirmed in the patients' medical records and be defined as any of the following: surgical hysterectomy with bilateral oophorectomy, bilateral tubular ligation, natural menopause with last menses >1 year ago; radiation induced oophorectomy with last menses >1 year ago; chemotherapy induced menopause with 1 year interval since last menses;
12. Ability to give written, informed consent prior to any study-specific Screening procedures, with the understanding that the consent may be withdrawn by the patient at any time without prejudice;
13. Capable of understanding the mandated and optional protocol requirements, is willing and able to comply with the study protocol procedures and has signed the main informed consent document. For any optional biopsy sampling (tissue and/or blood) and long-term sample storage, additional consent is required;

Exclusion Criteria
1. Pregnant or lactating;
2. Presence of an active uncontrolled infection or an unexplained fever greater than 38.5° C. during Screening up to the first scheduled day of dosing. At the discretion of the Investigator, patients with tumor fever or a clinically insignificant minor infection may be enrolled (i.e. mild upper respiratory infection);
3. Known hypersensitivity to any of the components of MF3958×MF3178 or history of severe hypersensitivity reactions to human or humanized monoclonal antibodies, including therapeutic antibodies;
4. Known HIV, active Hepatitis B or Hepatitis C; patients who have previously been treated for Hepatitis C and have undetectable viral loads are eligible;
5. Known symptomatic or unstable brain metastases. Patients with asymptomatic brain metastases are eligible to participate if the metastases have been radiographically and clinically stable for at least one month. If on steroids for this indication, the patient must be on a stable dose for at least one month.
6. Patients with leptomeningeal metastases;
7. Previous or concurrent malignancy, excluding non-basal cell carcinoma of skin or carcinoma in situ of the uterine cervix unless the tumor was treated with curative or palliative intent and in the opinion of the investigator, with sponsor agreement, previous or concurrent malignancy condition doesn't affect the assessment of safety and efficacy of the study drug;
8. Presence of NYHA Class III or IV congestive heart failure or LVEF <50% or history of significant cardiac disease, unstable angina, congestive heart failure, myocardial infarction, or ventricular arrhythmia requiring medication;
9. Presence of any other medical or psychological condition deemed by the Investigator to be likely to interfere with a patient's ability to sign informed consent, cooperate or participate in the study, or interfere with the interpretation of the results.

Statistical Analysis:

Part 1 and Part 2

Anti-tumor and clinical benefit variables are summarized descriptively for each group in Part 2. Where appropriate, variables are presented in terms of absolute and relative change from baseline. Categorical data is presented as percentages and frequency tabulations.

Where appropriate, data from those patients who receive what becomes identified as the MTD or the MRD during Part 1, and those receiving the same dose in Part 2, may be combined and summarized, as well as being summarized independently.

The frequency and nature of serious and non-serious AEs is assessed in absolute and relative frequencies and coded according to MedDRA medical dictionary.

Part 1

Data evaluation is descriptive in nature. Patient demographics, disease characteristics and pharmacokinetic and pharmacodynamic variables are summarized at each dose level. The frequency and nature of DLTs are also summarized at each dose level.

### Example 3

A patient having a solid tumor comprising ERBB2, ERBB3 positive expression and an applicable ERBB3 mutation was treated with MF3958×MF3178. This study shows that MF3958×MF3178 stabilizes the tumor in terms of size or lesions. The treatment prevents further tumor growth.

Tumor Molecular Profiling:

Analysis of tumor tissue shows an applicable ERBB3 mutation. In addition the tumor genome showed absence of mutations of KRAS, NRAS, PIK3CA, BRAF or the cell shows absence of mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, PTEN.

The tumor tissue having an ERBB3 mutation lacks the following oncogenic amplifications c-MET amplification, c-MYC amplification, EGFR amplification, ERBB2 amplification, MDM2 amplification.

The tumor tissue having an ERBB3 mutation does not have PTEN loss.

Treatment with the Bispecific Antibody MF3958×MF3178:

Treatment was given with bispecific antibody MF3958×MF3178 on a weekly regimen. The first dose was administered with premedication. In total 8 cycles were administered, during a study period of 8 months.

A weekly dose regimen with a 4-week cycle consists of a flat dose of 400 mg weekly for the first 2 cycles, with an 800 mg loading dose for the initial administration. From cycle 3, MF3958×MF3178 is given at a dose of 400 mg weekly for 3 weeks followed by 1 week off. Mandatory pre-medication is administered to mitigate IRRs. However, corticosteroids are only mandatory prior to the loading dose of Day 1 of Cycle 1 and should only be used for subsequent infusions as per the investigator's discretion to manage IRRs.

Efficacy:

The disease was assessed with measurable disease by using PET/CT-scan if possible or CT-scan. Anti-tumor activity and clinical benefit assessed by RECIST v1.1 determining objective overall response rate (ORR), duration of response (DOR), progression-free survival (PFS) and survival. Four tumor assessments reported stable disease (RECIST v1.1).

Alternatively to the dosing regimen of Example 3, medication can be provided in a bi-weekly dosing schedule as follows:

Bi-weekly schedule consisting of 750 mg of MF3958×MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

Alternatively to the dosing regimen of Example 3, medication can be provided in a tri-weekly dosing schedule as follows:

Tri-weekly schedule consisting of 750 mg of MF3958×MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

### Example 4

A 71-year-old male patient diagnosed with bladder cancer that carried mutation A232V in the ERRB3 protein was enrolled in the clinical trial study of Example 2 and 3. Treatment comprised a bi-weekly schedule consisting of 750 mg of MF3958×MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

### Example 5

A female patient diagnosed with ovarian clear cell carcinoma that carried mutation V104M in ERBB3 was enrolled in the clinical trial study of Example 2 and 3. Treatment comprised a bi-weekly schedule consisting of 750 mg of a bispecific antibody comprising binding arms MF3958 and MF3178 over four hours for the first infusion and then over two hours for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

Prior to starting administration of said bispecific antibody, the patient was admitted in the intensive care unit. Next to evaluating primary responses, secondary characteristics of clinically relevant activity were evaluated as part of the clinical trial study.

Clinical Activity:

The patient presented malignancy-related ascites due to ovarian cancer with extensive peritoneal metastases. Before initiating the treatment with said bispecific antibody, the patient needed large-volume abdominal paracentesis twice every week with new formation of ascites already observed the following day. After initiation treatment with said bispecific antibody, the patient reduced the requirements of therapeutic paracentesis to once a week with concomitant symptomatic improvement manifested by a better general performance status (from 3 to 2).

The following volumes of punctures were recorded at the indicated time intervals.

Three days after the first administration of the bispecific antibody, six liters of fluid were removed. After another four days, seven liters of fluid were removed and only after another six days, six liters were again removed.

The clinical and paraclinical observations are evident of therapeutic impact of administration of the bispecific antibody of the present invention.

1. A bispecific antibody that comprises an antigen binding site that can bind an extracellular part of ERBB2 and an antigen binding site that can bind an extracellular part of ERBB3 for use in a method of treatment of a cancer harboring a ERBB3 mutation in a subject in need thereof.

2. A method of treatment of a subject having a cancer harboring a ERBB3 mutation, the method comprising administering a bispecific antibody that comprises an antigen binding site that can bind an extracellular part of ERBB2 and an antigen binding site that can bind an extracellular part of ERBB3.

3. The method according to any one of the preceding claims, wherein the bispecific antibody comprises a first antigen-binding site that binds domain I of ERBB2 and a second antigen-binding site that binds domain III of ERBB3.

4. The method according to any one of the preceding claims, wherein the bispecific antibody comprises
i) at least the CDR1, CDR2 and CDR3 sequences of an ERBB2 specific heavy chain variable region selected from the group consisting of MF2926, MF2930, MF1849; MF2973, MF3004, MF3958, MF2971, MF3025, MF2916, MF3991, MF3031, MF2889, MF2913, MF1847, MF3001, MF3003 and MF1898 or wherein said antibody comprises CDR sequences that differ in at most 3 amino acids, preferably in at most 2 amino acids, preferably in at most 1 amino acid from the CDR1, CDR2 and CDR3 sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) at least the CDR1, CDR2 and CDR3 sequences of an ERBB3 specific heavy chain variable region selected from the group consisting of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises CDR sequences that differ in at most 3 amino acids, preferably in at most 2 amino acids, preferably in at most 1 amino acid from the CDR1, CDR2 and CDR3 sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

5. The method according to any one of the preceding claims, wherein the bispecific antibody comprises
i) an ERBB2 specific heavy chain variable region sequence selected from the group consisting of the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 and MF1898, or wherein said antibody comprises a heavy chain variable region sequence that differs in at most 15 amino acids from the heavy chain variable region sequences of MF2926, MF2930, MF1849; MF2973, MF3004, MF3958, MF2971, MF3025, MF2916, MF3991, MF3031, MF2889, MF2913, MF1847, MF3001, MF3003 or MF1898; and/or
ii) an ERBB3 specific heavy chain variable region sequence selected from the group consisting of the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 and MF6074, or wherein said antibody comprises a heavy chain variable region sequence that differs in at most 15 amino acids from the heavy chain variable region sequences of MF3178; MF3176; MF3163; MF3099; MF3307; MF6055; MF6056; MF6057; MF6058; MF6059; MF6060; MF6061; MF6062; MF6063; MF6064; MF 6065; MF6066; MF6067; MF6068; MF6069; MF6070; MF6071; MF6072; MF6073 or MF6074.

6. The method according to any one of the preceding claims, wherein the bispecific antibody comprises heavy chain variable regions MF3958 and MF3178.

7. The method according to any one of the preceding claims, wherein the bispecific antibody comprises a variable domain that comprises said first antigen binding site and the variable domain that comprises said second antigen binding site of said bispecific antibody preferably comprise a light chain variable region comprising a CDR1 having the sequence (RASQSISSYLN), a CDR2 having the sequence (AASSLQS), and a CDR3 having the sequence (QQSYSTPPT), according to KABAT numbering or according to the IMGT numbering system, the CDRs of said light chain variable region are QSISSY, AAS and QQSYSTPPT, respectively.

8. The method according to any one of the preceding claims, wherein the cancer harboring a ERBB3 mutation is colorectal cancer, gastric cancer, non-small-cell lung cancer (NSCLC) adenocarcinoma (adeno), NSCLC squamous cell carcinoma, renal carcinoma, melanoma, ovarian cancer, lung large cell carcinoma, esophageal cancer, small-cell lung cancer, hepatocellular cancer (HCC), breast cancer, hormone-positive breast cancer, glioblastoma, and head and neck cancer, gastric adenocarcinoma or an esophago-gastric cancer, a pancreatic cancer, a pancreatic ductal adenocarcinoma, a sarcoma, a bladder cancer, a gallbladder cancer, head and neck cancer, a prostate cancer, uterine/endometrial cancer, a lung cancer such as non-small cell lung cancer, including invasive mucinous adenocarcinoma.

9. The method according to any one of the preceding claims, wherein the ERBB3 mutation comprises a mutation in domain I, II, III or IV of ERBB3.

10. The method according to any one of the preceding claims, wherein the ERBB3 mutation comprises a mutation in the intracellular domain, preferably in the tyrosine kinase domain. preferably involving an amino acid residue in the range of 710-964, preferably at positions Q809, S846, Q865 or E928.

11. The method according to any one of the preceding claims, wherein the ERBB3 mutation comprises a mutation which causes ligand independent heterodimerization of ERBB2 and ERBB3.

12. The method according to claim 11, wherein the ERBB3 mutation promotes the PI3K pathway.

13. The method according to any one of the preceding claims, wherein the ERBB3 mutation comprises a mutation in the extracellular domain of ERBB3.

14. The method according to any one of the preceding claims, wherein the ERBB3 mutation comprises a mutation in the intracellular domain of ERBB3.

15. The method according to any one of the preceding claims, wherein a mutation in ERBB3 is a mutation over the non-mutated sequence according to SEQ ID No: 1.

16. The method according to any one of the preceding claims, wherein the ERBB3 mutation comprises one or more of the following mutations
M60N, where N is any naturally occurring amino acid, preferably K;
M91N, where N is any naturally occurring amino acid, preferably I;
R103N, where N is any naturally occurring amino acid, preferably G;
V104N, where N is any naturally occurring amino acid, preferably L or M;
R135N, where N is any naturally occurring amino acid, preferably C;
F219N, where N is any naturally occurring amino acid, preferably L;
H228N, where N is any naturally occurring amino acid, preferably Q;
A232N, where N is any naturally occurring amino acid, preferably V;
P262N, where N is any naturally occurring amino acid, preferably H or L or S;
G284N, where N is any naturally occurring amino acid, preferably R;
D297N, where N is any naturally occurring amino acid, preferably Y or A or H or N or V;
K329N, where N is any naturally occurring amino acid, preferably E or I or T;
E332N, where N is any naturally occurring amino acid, preferably K;
T355N, where N is any naturally occurring amino acid, preferably A or I or P;
R475N, where N is any naturally occurring amino acid, preferably W;
Q809N, where N is any naturally occurring amino acid, preferably R;
S846N, where N is any naturally occurring amino acid, preferably I;
Q865N, where N is any naturally occurring amino acid, preferably H;
E928N, where N is any naturally occurring amino acid, preferably G.

17. The method according to any one of the preceding claims, wherein the ERBB3 does not have a mutation at R426.

18. The method according to any one of the preceding claims, wherein the cancer lacks detectable mutations in one or more genes selected from the group including BRAF, EGFR, KRAS, cKIT-BRCA1-2, MET, ROS, RET, ALK, AKT1, ERBB4, NFE2L2, PTPN11, FBXW7, NRAS, RHOA, CTNNB1, HRAS, SF3B1, DICER1, KIT, PIK3CA, PIK3R1, SMAD4, PPP2R1A, VHL, ERBB2, MTOR, and PTEN.

19. The method according to any one of the preceding claims, wherein the cancer lacks detectable amplifications in one or more genes selected from the group including c-MET, c-MYC, EGFR, ERBB2, and MDM2 amplification.

20. The method according to any one of the preceding claims, wherein the cancer does not have a detectable PTEN loss.

21. The method according to any one of the preceding claims, wherein the administration of the bispecific antibody comprises a first line therapy for treatment of a cancer harboring a ERBB3 mutation in said subject.

22. The method according to claims 1 to 20, wherein the cancer has progressed after the subject having received a prior treatment.

23. The method of claim 22, wherein the prior treatment comprises chemotherapy, checkpoint inhibitor therapy (including anti-PD1 and anti-PD-L1 approved therapies and applicable therapies in clinical development), anti-ERBB2 or anti-ERBB3 or anti-VEGFR2 (vascular endothelial growth factor receptor 2), or a prior treatment with a tyrosine kinase inhibitor (TKI) of ERBB2 or of VEGFR2-TIE2, or with a combination of TKIs or any of the above.

24. The method of claim 23, wherein the chemotherapy comprises gemcitabine, capecitabine, carboplatin, a taxane, such as docetaxel or paclitaxel, 5-fluorouracil (with or without radiotherapy), vinorelbine, carmustine, doxorubicin, epirubicin, mitoxantrone, vinblastine, cisplatin (or pemetrexed), oxaliplatin, carboplatin, ifosfamide, mytomycin C, vindesine, etoposide, Folfox (i.e. a combination of 5-fluorouracil, leucovorin, and oxaliplatin) or Folfiri (i.e. a combination of leucovorin, 5-fluorouracil and irinotecan), Folfirinox (a combination of leucovorin, 5-fluorouracil, irinotecan and oxaliplatin) or any combination thereof.

25. The method of claim 23, wherein the checkpoint inhibitor therapy according to the present invention includes an anti-PD1, anti-PD-L1, and anti-CTLA4 therapeutic moiety and preferably comprises ipilimumab, nivolumab, pembrolizumab, atezolizumab, avelumab, durvalumab or cemiplimab, or any combination thereof.

26. The method of claim 23, wherein the TKI is lapatinib, canertinib, neratinib, tucatinib (irbinitinib), CP-724714, tarloxitinib, mubritinib, afatinib, varlitinib, and dacomitinib, afatinib, or any combination thereof.

27. The method of claim 23, wherein the VEGFR2 targeted treatment is ramucirumab or regorafenib.

28. The method according to any one of the preceding claims, wherein the cancer comprises mutation A232V in the ERBB3 protein.

29. The method according to any one of the preceding claims, wherein the cancer is bladder cancer comprising mutation A232V in the ERBB3 protein.

30. The method according to any one of the preceding claims, wherein the cancer comprises mutation V104M in the ERBB3 protein.

31. The method according to any one of the preceding claims, wherein the cancer is ovarian cancer, such as clear cell carcinoma, comprising mutation V104M in the ERBB3 protein.


SEQUENCE LISTING (233 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 11 aa, Artificial Sequence
  VL CDR1
RASQSISSYLN

SEQ ID NO: 3 - protein, 7 aa, Artificial Sequence
  VL CDR2
AASSLQS

SEQ ID NO: 4 - protein, 9 aa, Artificial Sequence
  VL CDR3
QQSYSTPPT

SEQ ID NO: 5 - protein, 6 aa, Artificial Sequence
  IMGT VL CDR1
QSISSY

SEQ ID NO: 6 - protein, 9 aa, Artificial Sequence
  IMGT VL CDR3
QQSYSTPPT

SEQ ID NO: 7 - protein, 214 aa, Artificial Sequence
  common light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 321 bp, Artificial Sequence
  common light chain variable region
  CDS (1)..(321)
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 9 - protein, 107 aa, Artificial Sequence
  Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 10 - DNA, 324 bp, Artificial Sequence
  common light chain constant region
  CDS (1)..(324)
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - protein, 107 aa, Artificial Sequence [sequence omitted]

SEQ ID NO: 12 - protein, 107 aa, Artificial Sequence; identical to SEQ ID NO: 9
  IGKV1-39/jk5 common light chain variable region

SEQ ID NO: 13 - protein, 95 aa, Artificial Sequence
  V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 14 - DNA, 294 bp, Artificial Sequence
  IgG heavy chain CH1 region
  CDS (1)..(294)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 15 - protein, 98 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 16 - DNA, 45 bp, Artificial Sequence
  IgG heavy chain hinge region
  CDS (1)..(45)
gagcccaaatcttgtgacaaaactcacacatgcccaccgtgccca

SEQ ID NO: 17 - protein, 15 aa, Artificial Sequence
  Synthetic Construct
EPKSCDKTHTCPPCP

SEQ ID NO: 18 - DNA, 330 bp, Artificial Sequence
  IgG heavy chain CH2 region
  CDS (1)..(330)
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 19 - protein, 110 aa, Artificial Sequence
  Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 20 - DNA, 321 bp, Artificial Sequence
  IgG heavy chain CH3 domain containing variations L351K and T366K
  CDS (1)..(321)
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 21 - protein, 106 aa, Artificial Sequence
  Synthetic Construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - DNA, 321 bp, Artificial Sequence
  IgG heavy chain CH3 domain containing variations L351D and L368E
  CDS (1)..(321)
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 23 - protein, 106 aa, Artificial Sequence
  Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 24 - DNA, 370 bp, Artificial Sequence
  MF2926
ggcccagccggccatggcccaggtccagctgcagcagtctggacctgagctggtgaaacctggggcttcagtgatgatttcctgcaaggcttctggttactcattcactggctaccacatgaactgggtgaagcaaagtcctgaaaagagccttgagtggattggagacataaatcctagcattggtacgactgcccacaaccagattttcagggccaaggccacaatgactgttgacaaatcctccaacacagcctacatgcagctcaagagcctgacatctgaagactctggagtcttttactgtgttagaagaggggactggtccttcgatgtctggggcacagggaccacggtcaccgtctccagt

SEQ ID NO: 25 - protein, 117 aa, Artificial Sequence
  MF2926
QVQLQQSGPELVKPGASVMISCKASGYSFTGYHMNWVKQSPEKSLEWIGDINPSIGTTAHNQIFRAKATMTVDKSSNTAYMQLKSLTSEDSGVFYCVRRGDWSFDVWGTGTTVTVSS

SEQ ID NO: 26 - protein, 5 aa, Artificial Sequence
  MF2926 CDR1
GYHMN

SEQ ID NO: 27 - protein, 17 aa, Artificial Sequence
  MF2926 CDR2
DINPSIGTTAHNQIFRA

SEQ ID NO: 28 - protein, 8 aa, Artificial Sequence
  MF2926 CDR3
RGDWSFDV

SEQ ID NO: 29 - DNA, 379 bp, Artificial Sequence
  MF2930
ggcccagccggccatggccgaggtccagctgcagcagtctggggctgaactggtgaagcctggagcctcagtgatgatgtcctgtaaggtttctggctacaccttcacttcctatcctatagcgtggatgaagcaggttcatggaaagagcctagagtggattggaaattttcatccttacagtgatgatactaagtacaatgaaaacttcaagggcaaggccacattgactgtagaaaaatcctctagcacagtctacttggagctcagccgattaacatctgatgactctgctgtttattactgtgcaagaagtaacccattatattactttgctatggactactggggtcaaggaacctcggtcaccgtctccagt

SEQ ID NO: 30 - protein, 120 aa, Artificial Sequence
  MF2930
EVQLQQSGAELVKPGASVMMSCKVSGYTFTSYPIAWMKQVHGKSLEWIGNFHPYSDDTKYNENFKGKATLTVEKSSSTVYLELSRLTSDDSAVYYCARSNPLYYFAMDYWGQGTSVTVSS

SEQ ID NO: 31 - protein, 5 aa, Artificial Sequence
  MF2930 CDR1
SYPIA

SEQ ID NO: 32 - protein, 17 aa, Artificial Sequence
  MF2930 CDR2
NFHPYSDDTKYNENFKG

SEQ ID NO: 33 - protein, 11 aa, Artificial Sequence
  MF2930 CDR3
SNPLYYFAMDY

SEQ ID NO: 34 - DNA, 385 bp, Artificial Sequence
  MF1849
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaaggtgactacggttcttactcttcttacgcctttgattattggggccaaggtaccctggtcaccgtctccagt

SEQ ID NO: 35 - protein, 122 aa, Artificial Sequence
  MF1849
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGDYGSYSSYAFDYWGQGTLVTVSS

SEQ ID NO: 36 - protein, 5 aa, Artificial Sequence
  MF1849 CDR1
SYGMH

SEQ ID NO: 37 - protein, 17 aa, Artificial Sequence
  MF1849 CDR2
VISYDGSNKYYADSVKG

SEQ ID NO: 38 - protein, 13 aa, Artificial Sequence
  MF1849 CDR3
GDYGSYSSYAFDY

SEQ ID NO: 39 - DNA, 385 bp, Artificial Sequence
  MF2973
ggcccagccggccatggcccaggtgcagctgaagcagtctggggctgagctggtgaggcctggggcttcagtgaagttgtcctgcaaggcttctggctacattttcactggctactatataaactggttgaggcagaggcctggacagggacttgaatggattgcaaaaatttatcctggaagtggtaatacttactacaatgagaagttcaggggcaaggccacactgactgcagaagaatcctccagcactgcctacatgcagctcagcagcctgacatctgaggactctgctgtctatttctgtgcaagagggccccactatgattacgacggcccctggtttgtttactggggccaagggactctggtcaccgtctccagt

SEQ ID NO: 40 - protein, 122 aa, Artificial Sequence
  MF2973
QVQLKQSGAELVRPGASVKLSCKASGYIFTGYYINWLRQRPGQGLEWIAKIYPGSGNTYYNEKFRGKATLTAEESSSTAYMQLSSLTSEDSAVYFCARGPHYDYDGPWFVYWGQGTLVTVSS

SEQ ID NO: 41 - protein, 5 aa, Artificial Sequence
  MF2973 CDR1
GYYIN

SEQ ID NO: 42 - protein, 17 aa, Artificial Sequence
  MF2973 CDR2
KIYPGSGNTYYNEKFRG

SEQ ID NO: 43 - protein, 13 aa, Artificial Sequence
  MF2973 CDR3
GPHYDYDGPWFVY

SEQ ID NO: 44 - DNA, 382 bp, Artificial Sequence
  MF3004
ggcccagccggccatggcccaggtgcagctgaagcagtctggggctgagctggtgaggcctggggcttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccagcactgcctacatgcacctcagcagcctgacatctgaggactctgctgtctatttctgtgcaagaccccactatggttacgacgactggtacttcggtgtctggggcacaggcaccacggtcaccgtctccagt

SEQ ID NO: 45 - protein, 121 aa, Artificial Sequence
  MF3004
QVQLKQSGAELVRPGASVKLSCKASGYTFTGYYINWVKQRPGQGLEWIARIYPGSGYTYYNEKFKGKATLTAEESSSTAYMHLSSLTSEDSAVYFCARPHYGYDDWYFGVWGTGTTVTVSS

SEQ ID NO: 46 - protein, 5 aa, Artificial Sequence
  MF3004 CDR1
GYYIN

SEQ ID NO: 47 - protein, 17 aa, Artificial Sequence
  MF3004 CDR2
RIYPGSGYTYYNEKFKG

SEQ ID NO: 48 - protein, 12 aa, Artificial Sequence
  MF3004 CDR3
PHYGYDDWYFGV

SEQ ID NO: 49 - DNA, 382 bp, Artificial Sequence
  MF2971
ggcccagccggccatggcccaggtgcagctgaagcagtctggggctgagctggtgaggcctggggcttcagtgaaactgtcctgcaaggcttctggctacactttcactgcctactatataaactgggtgaagcagaggcctggacagggacttgagtggattgcaaggatttatcctggaagtggctatacttactacaatgagattttcaagggcagggccacactgactgcagacgaatcctccagcactgcctacatgcaactcagcagcctgacatctgaggactctgctgtctatttctgtgcaagacctccggtctactatgactcggcctggtttgcttactggggccaagggactctggtcaccgtctccagt

SEQ ID NO: 50 - protein, 121 aa, Artificial Sequence
  MF2971
QVQLKQSGAELVRPGASVKLSCKASGYTFTAYYINWVKQRPGQGLEWIARIYPGSGYTYYNEIFKGRATLTADESSSTAYMQLSSLTSEDSAVYFCARPPVYYDSAWFAYWGQGTLVTVSS

SEQ ID NO: 51 - protein, 5 aa, Artificial Sequence
  MF2971 CDR1
AYYIN

SEQ ID NO: 52 - protein, 17 aa, Artificial Sequence
  MF2971 CDR2
RIYPGSGYTYYNEIFKG

SEQ ID NO: 53 - protein, 12 aa, Artificial Sequence
  MF2971 CDR3
PPVYYDSAWFAY

SEQ ID NO: 54 - DNA, 382 bp, Artificial Sequence
  MF3025
ggcccagccggccatggcccaggtgcagctgaagcagtctggggctgagctggtgaggcctgggacttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccaacactgcctatatgcacctcagcagcctgacatctgaggactctgctgtctatttctgtgcaaggccccactatggttacgacgactggtacttcgctgtctggggcacagggaccacggtcaccgtctccagt

SEQ ID NO: 55 - protein, 121 aa, Artificial Sequence
  MF3025
QVQLKQSGAELVRPGTSVKLSCKASGYTFTGYYINWVKQRPGQGLEWIARIYPGSGYTYYNEKFKGKATLTAEESSNTAYMHLSSLTSEDSAVYFCARPHYGYDDWYFAVWGTGTTVTVSS

SEQ ID NO: 56 - protein, 5 aa, Artificial Sequence
  MF3025 CDR1
GYYIN

SEQ ID NO: 57 - protein, 17 aa, Artificial Sequence
  MF3025 CDR2
RIYPGSGYTYYNEKFKG

SEQ ID NO: 58 - protein, 12 aa, Artificial Sequence
  MF3025 CDR3
PHYGYDDWYFAV

SEQ ID NO: 59 - DNA, 385 bp, Artificial Sequence
  MF2916
ggcccagccggccatggcccaggtccagctgcagcagtctggggctgagctggtgaggcctggggcttcagtgaagctgtcctgcaaggcttctggctacactttcactggctactatataaactgggtgaagcagaggcctggacagggacttgagtggattgcaaggatttatcctggcagtggtcatacttcctacaatgagaagttcaagggcaaggccacactgactacagaaaaatcctccagcactgcctacatgcagctcagcagcctgacatctgaggactctgctgtctatttctgtgcaagacctatctactttgattacgcaggggggtacttcgatgtctggggcacaagaacctcggtcaccgtctccagt

SEQ ID NO: 60 - protein, 122 aa, Artificial Sequence
  MF2916
QVQLQQSGAELVRPGASVKLSCKASGYTFTGYYINWVKQRPGQGLEWIARIYPGSGHTSYNEKFKGKATLTTEKSSSTAYMQLSSLTSEDSAVYFCARPIYFDYAGGYFDVWGTRTSVTVSS

SEQ ID NO: 61 - protein, 5 aa, Artificial Sequence
  MF2916 CDR1
GYYIN

SEQ ID NO: 62 - protein, 17 aa, Artificial Sequence
  MF2916 CDR2
RIYPGSGHTSYNEKFKG

SEQ ID NO: 63 - protein, 13 aa, Artificial Sequence
  MF2916 CDR3
PIYFDYAGGYFDV

SEQ ID NO: 64 - DNA, 382 bp, Artificial Sequence
  MF3958
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccccgtgtactacgacagcgcttggtttgcctactggggccagggcaccctggtcaccgtctccagt

SEQ ID NO: 65 - protein, 121 aa, Artificial Sequence
  MF3958
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSS

SEQ ID NO: 66 - protein, 5 aa, Artificial Sequence
  MF3958 CDR1
AYYIN

SEQ ID NO: 67 - protein, 17 aa, Artificial Sequence
  MF3958 CDR2
RIYPGSGYTSYAQKFQG

SEQ ID NO: 68 - protein, 12 aa, Artificial Sequence
  MF3958 CDR3
PPVYYDSAWFAY

SEQ ID NO: 69 - DNA, 382 bp, Artificial Sequence
  MF3031
ggcccagccggccatggcccaggtccagctgcagcagtctggggctgagctggtgaggcctggggcttcagtgaagctgtcctgcaaggcttctggctacactttcactgcctactatataaactgggtgaagcagaggcctggacagggacttgagtggattgcaaagatttatcctggaagtggttatacttactacaatgagaatttcaggggcaaggccacactgactgcagaagaatcctccagtactgcctacatacaactcagcagcctgacatctgaggactctgctgtctatttctgtgcaagaggcgtctatgattacgacggggcctggtttgcttactggggccaagggactctggtcaccgtctccagt

SEQ ID NO: 70 - protein, 121 aa, Artificial Sequence
  MF3031
QVQLQQSGAELVRPGASVKLSCKASGYTFTAYYINWVKQRPGQGLEWIAKIYPGSGYTYYNENFRGKATLTAEESSSTAYIQLSSLTSEDSAVYFCARGVYDYDGAWFAYWGQGTLVTVSS

SEQ ID NO: 71 - protein, 5 aa, Artificial Sequence
  MF3031 CDR1
AYYIN

SEQ ID NO: 72 - protein, 17 aa, Artificial Sequence
  MF3031 CDR2
KIYPGSGYTYYNENFRG

SEQ ID NO: 73 - protein, 12 aa, Artificial Sequence
  MF3031 CDR3
GVYDYDGAWFAY

SEQ ID NO: 74 - DNA, 382 bp, Artificial Sequence
  MF3991
ggcccagccggccatggcccaggtgcagctggtgcagtctggcgccgaagtgaagaaacctggcgccagcgtgaagctgagctgcaaggccagcggctacaccttcaccgcctactacatcaactgggtccgacaggccccaggccagggcctggaatggatcggcagaatctaccccggctccggctacaccagctacgcccagaagttccagggcagagccaccctgaccgccgacgagagcaccagcaccgcctacatggaactgagcagcctgcggagcgaggataccgccgtgtacttctgcgccagaccccactacggctacgacgactggtacttcggcgtgtggggccagggcaccctggtcaccgtctccagt

SEQ ID NO: 75 - protein, 121 aa, Artificial Sequence
  MF3991
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPHYGYDDWYFGVWGQGTLVTVSS

SEQ ID NO: 76 - protein, 5 aa, Artificial Sequence
  MF3991 CDR1
AYYIN

SEQ ID NO: 77 - protein, 17 aa, Artificial Sequence
  MF3991 CDR2
RIYPGSGYTSYAQKFQG

SEQ ID NO: 78 - protein, 12 aa, Artificial Sequence
  MF3991 CDR3
PHYGYDDWYFGV

SEQ ID NO: 79 - DNA, 379 bp, Artificial Sequence
  MF2889
ggcccagccggccatggccgaggtccagctgcagcagtctggagctgagctggtaaggcctgggacttcagtgaaggtgtcctgcaaggcttctggatacgccttcactaattatttgatagagtgggtaaagcagaggcctggccagggccttgagtggattggagtgatttatcctgaaggtggtggtactatctacaatgagaagttcaagggcaaggcaacactgactgcagacaaatcctccagcactgcctacatgcagctcagcggcctgacatctgaggactctgcggtctatttctgtgcaagaggagactatgattacaaatatgctatggactactggggtcaaggaacctcggtcaccgtctccagt

SEQ ID NO: 80 - protein, 120 aa, Artificial Sequence
  MF2889
EVQLQQSGAELVRPGTSVKVSCKASGYAFTNYLIEWVKQRPGQGLEWIGVIYPEGGGTIYNEKFKGKATLTADKSSSTAYMQLSGLTSEDSAVYFCARGDYDYKYAMDYWGQGTSVTVSS

SEQ ID NO: 81 - protein, 5 aa, Artificial Sequence
  MF2889 CDR1
NYLIE

SEQ ID NO: 82 - protein, 17 aa, Artificial Sequence
  MF2889 CDR2
VIYPEGGGTIYNEKFKG

SEQ ID NO: 83 - protein, 11 aa, Artificial Sequence
  MF2889 CDR3
GDYDYKYAMDY

SEQ ID NO: 84 - DNA, 370 bp, Artificial Sequence
  MF2913
ggcccagccggccatggccgaggtcaagctgcagcagtctggacctgagctggtgaagcctggcgcttcagtgaagatatcctgcaaggcttctggttactcattcactgactacaaaatggactgggtgaagcagagccatggaaagagcctcgaatggattggaaatattaatcctaacagtggtggtgttatctacaaccagaagttcaggggcaaggtcacattgactgttgacaggtcctccagcgcagcctacatggagctccgcagcctgacatctgaggacactgcagtctattattgttcaagaggactgtgggatgctatggactcctggggtcaaggaacctcggtcaccgtctccagt

SEQ ID NO: 85 - protein, 117 aa, Artificial Sequence
  MF2913
EVKLQQSGPELVKPGASVKISCKASGYSFTDYKMDWVKQSHGKSLEWIGNINPNSGGVIYNQKFRGKVTLTVDRSSSAAYMELRSLTSEDTAVYYCSRGLWDAMDSWGQGTSVTVSS

SEQ ID NO: 86 - protein, 5 aa, Artificial Sequence
  MF2913 CDR1
DYKMD

SEQ ID NO: 87 - protein, 17 aa, Artificial Sequence
  MF2913 CDR2
NINPNSGGVIYNQKFRG

SEQ ID NO: 88 - protein, 8 aa, Artificial Sequence
  MF2913 CDR3
GLWDAMDS

SEQ ID NO: 89 - DNA, 382 bp, Artificial Sequence
  MF1847
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaaggttggtggcatccgctgctgtctggctttgattattggggccaaggtaccctggtcaccgtctccagt

SEQ ID NO: 90 - protein, 121 aa, Artificial Sequence
  MF1847
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGWWHPLLSGFDYWGQGTLVTVSS

SEQ ID NO: 91 - protein, 5 aa, Artificial Sequence
  MF1847 CDR1
SYGMH

SEQ ID NO: 92 - protein, 17 aa, Artificial Sequence
  MF1847 CDR2
VISYDGSNKYYADSVKG

SEQ ID NO: 93 - protein, 12 aa, Artificial Sequence
  MF1847 CDR3
GWWHPLLSGFDY

SEQ ID NO: 94 - DNA, 370 bp, Artificial Sequence
  MF3001
ggcccagccggccatggccgaggtccagctgcagcagtctggggctgaactggcaaaacctggggcctcagtgaagctgtcctgcaagacttctggctacaactttcctatctactggatgcactgggtaaaacagaggcctggacggggtctggaatggattggatacattaatcctagtactggttatattaagaacaatcagaagttcaaggacaaggccaccttgactgcagacaaatcctccaacacagcctacatgcagctgaacagcctgacatatgaggactctgcagtctattactgtacaagagaagggataactgggtttacttactggggccaagggactctggtcaccgtctccagt

SEQ ID NO: 95 - protein, 117 aa, Artificial Sequence
  MF3001
EVQLQQSGAELAKPGASVKLSCKTSGYNFPIYWMHWVKQRPGRGLEWIGYINPSTGYIKNNQKFKDKATLTADKSSNTAYMQLNSLTYEDSAVYYCTREGITGFTYWGQGTLVTVSS

SEQ ID NO: 96 - protein, 5 aa, Artificial Sequence
  MF3001 CDR1
IYWMH

SEQ ID NO: 97 - protein, 17 aa, Artificial Sequence
  MF3001 CDR2
YINPSTGYIKNNQKFKD

SEQ ID NO: 98 - protein, 8 aa, Artificial Sequence
  MF3001 CDR3
EGITGFTY

SEQ ID NO: 99 - DNA, 385 bp, Artificial Sequence
  MF1898
ggcccagccggccatggcccaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaagatggtttccgtcgtactactctgtctggctttgattattggggccaaggtaccctggtcaccgtctccagt

SEQ ID NO: 100 - protein, 122 aa, Artificial Sequence
  MF1898
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDGFRRTTLSGFDYWGQGTLVTVSS

SEQ ID NO: 101 - protein, 5 aa, Artificial Sequence
  MF1898 CDR1
SYGMH

SEQ ID NO: 102 - protein, 17 aa, Artificial Sequence
  MF1898 CDR2
VISYDGSNKYYADSVKG

SEQ ID NO: 103 - protein, 13 aa, Artificial Sequence
  MF1898 CDR3
DGFRRTTLSGFDY

SEQ ID NO: 104 - DNA, 379 bp, Artificial Sequence
  MF3003
ggcccagccggccatggcccaggtgcagctgaagcagtctggacctgagctggtgaagcctggggcctcagtgaagatttcctgcaaggcttctggcgacgcattcagttactcctggatgaactgggtgaagcagaggcctggaaagggtcttgagtggattggacggatttatcctggagatggagatattaactacaatgggaagttcaagggcaaggccacactgactgcagacaaatcctccagcacagcccacctgcaactcaacagcctgacatctgaggactctgcggtctacttctgtgcaagaggacagctcggactagaggcctggtttgcttattggggccaggggactctggtcaccgtctccagt

SEQ ID NO: 105 - protein, 120 aa, Artificial Sequence
  MF3003
QVQLKQSGPELVKPGASVKISCKASGDAFSYSWMNWVKQRPGKGLEWIGRIYPGDGDINYNGKFKGKATLTADKSSSTAHLQLNSLTSEDSAVYFCARGQLGLEAWFAYWGQGTLVTVSS

SEQ ID NO: 106 - protein, 5 aa, Artificial Sequence
  MF3003 CDR1
YSWMN

SEQ ID NO: 107 - protein, 17 aa, Artificial Sequence
  MF3003 CDR2
RIYPGDGDINYNGKFKG

SEQ ID NO: 108 - protein, 11 aa, Artificial Sequence
  MF3003 CDR3
GQLGLEAWFAY

SEQ ID NO: 109 - DNA, 391 bp, Artificial Sequence
  MF3178
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 110 - protein, 124 aa, Artificial Sequence
  MF3178
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 111 - protein, 5 aa, Artificial Sequence
  MF3178 CDR1
GYYMH

SEQ ID NO: 112 - protein, 17 aa, Artificial Sequence
  MF3178 CDR2
WINPNSGGTNYAQKFQG

SEQ ID NO: 113 - protein, 15 aa, Artificial Sequence
  MF3178 CDR3
DHGSRHFWSYWGFDY

SEQ ID NO: 114 - DNA, 385 bp, Artificial Sequence
  MF3176
ggcccagccggccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggctgtgtattactgtgcaagagattggtggtacccgccgtactactggggctttgattattggggccaaggtaccctggtcaccgtctccagt

SEQ ID NO: 115 - protein, 122 aa, Artificial Sequence
  MF3176
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWWYPPYYWGFDYWGQGTLVTVSS

SEQ ID NO: 116 - protein, 5 aa, Artificial Sequence
  MF3176 CDR1
SYAMS

SEQ ID NO: 117 - protein, 17 aa, Artificial Sequence
  MF3176 CDR2
AISGSGGSTYYADSVKG

SEQ ID NO: 118 - protein, 13 aa, Artificial Sequence
  MF3176 CDR3
DWWYPPYYWGFDY

SEQ ID NO: 119 - DNA, 391 bp, Artificial Sequence
  MF3163
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaaaagattcttactctcgtcatttctactcttggtgggcctttgattattggggccaaggtaccctggtcaccgtctccagt

SEQ ID NO: 120 - protein, 124 aa, Artificial Sequence
  MF3163
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAKDSYSRHFYSWWAFDYWGQGTLVTVSS

SEQ ID NO: 121 - protein, 5 aa, Artificial Sequence
  MF3163 CDR1
GYYMH

SEQ ID NO: 122 - protein, 17 aa, Artificial Sequence
  MF3163 CDR2
WINPNSGGTNYAQKFQG

SEQ ID NO: 123 - protein, 15 aa, Artificial Sequence
  MF3163 CDR3
DSYSRHFYSWWAFDY

SEQ ID NO: 124 - DNA, 382 bp, Artificial Sequence
  MF3099
ggcccagccggccatggccgaggtccagctgcagcagcctggggctgagctggtgaggcctgggacttcagtgaagttgtcctgcaaggcttctggctacaccttcaccagctactggatgcactgggtaaagcagaggcctggacaaggccttgagtggatcggaattcttgatccttctgatagttatactacctacaatcaaaagttcaagggcaaggccacattaacagtagacacatcctccagcatagcctacatgcagctcagcagcctgacatctgaggactctgcgctctattactgtgcaagagggggagattacgacgagggaggtgctatggactactggggtcaaggaacctcggtcaccgtctccagt

SEQ ID NO: 125 - protein, 121 aa, Artificial Sequence
  MF3099
EVQLQQPGAELVRPGTSVKLSCKASGYTFTSYWMHWVKQRPGQGLEWIGILDPSDSYTTYNQKFKGKATLTVDTSSSIAYMQLSSLTSEDSALYYCARGGDYDEGGAMDYWGQGTSVTVSS

SEQ ID NO: 126 - protein, 5 aa, Artificial Sequence
  MF3099 CDR1
SYWMH

SEQ ID NO: 127 - protein, 17 aa, Artificial Sequence
  MF3099 CDR2
ILDPSDSYTTYNQKFKG

SEQ ID NO: 128 - protein, 12 aa, Artificial Sequence
  MF3099 CDR3
GGDYDEGGAMDY

SEQ ID NO: 129 - DNA, 391 bp, Artificial Sequence
  MF3307
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaagaggttctcgtaaacgtctgtctaactacttcaacgcctttgattattggggccaaggtaccctggtcaccgtctccagt

SEQ ID NO: 130 - protein, 124 aa, Artificial Sequence
  MF3307
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSRKRLSNYFNAFDYWGQGTLVTVSS

SEQ ID NO: 131 - protein, 5 aa, Artificial Sequence
  MF3307 CDR1
GYYMH

SEQ ID NO: 132 - protein, 17 aa, Artificial Sequence
  MF3307 CDR2
WINPNSGGTNYAQKFQG

SEQ ID NO: 133 - protein, 15 aa, Artificial Sequence
  MF3307 CDR3
GSRKRLSNYFNAFDY

SEQ ID NO: 134 - DNA, 391 bp, Artificial Sequence
  MF6058
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaaccctcaaagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcaccgtctccagt

SEQ ID NO: 135 - protein, 124 aa, Artificial Sequence
  MF6058
QVQLVQSGADVKKPGASVKVTCKASGYTFTGYYMHWVRQAPGQALEWMGWINPQSGGTNYAKKFQGRVSMTRETSTSTAYMQLSRLRSDDTATYYCARDHGSRHFWSYWGFDYWGQGTLVTVSS

SEQ ID NO: 136 - protein, 5 aa, Artificial Sequence
  MF6058 CDR1
GYYMH

SEQ ID NO: 137 - protein, 17 aa, Artificial Sequence
  MF6058 CDR2
WINPQSGGTNYAKKFQG

SEQ ID NO: 138 - protein, 15 aa, Artificial Sequence
  MF6058 CDR3
DHGSRHFWSYWGFDY

SEQ ID NO: 139 - DNA, 391 bp, Artificial Sequence
  MF6061
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttaagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 140 - protein, 124 aa, Artificial Sequence
  MF6061
QVQLVQSGAEVKKPGASVKVSCKASGYTFTGYYMHWVRQAPGQGLEWMGWINPQSGGTNYAQKFKGRVTMTRDTSTSTAYMELSRLRSDDTAVYYCARDHGSRHFWSYWGFDYWGQGTLVTVSS

SEQ ID NO: 141 - protein, 5 aa, Artificial Sequence
  MF6061 CDR1
GYYMH

SEQ ID NO: 142 - protein, 17 aa, Artificial Sequence
  MF6061 CDR2
WINPQSGGTNYAQKFKG

SEQ ID NO: 143 - protein, 15 aa, Artificial Sequence
  MF6061 CDR3
DHGSRHFWSYWGFDY

SEQ ID NO: 144 - DNA, 391 bp, Artificial Sequence
  MF6065
ggcccagccggccatggcccaggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaaccctcaggggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcaccgtctccagt

SEQ ID NO: 145 - protein, 124 aa, Artificial Sequence
  MF6065
QVQLVQSGAEVKKPGASVKVSCKASGYTFTSYYMHWVRQAPGQGLEWMGWINPQGGSTNYAQKFQGRVTMTRDTSTSTVYMELSRLRSEDTAVYYCARDHGSRHFWSYWGFDYWGQGTLVTVSS

SEQ ID NO: 146 - protein, 5 aa, Artificial Sequence
  MF6065 CDR1
SYYMH

SEQ ID NO: 147 - protein, 17 aa, Artificial Sequence
  MF6065 CDR2
WINPQGGSTNYAQKFQG

SEQ ID NO: 148 - protein, 15 aa, Artificial Sequence
  MF6065 CDR3
DHGSRHFWSYWGFDY

SEQ ID NO: 149 - DNA, 382 bp, Artificial Sequence
  MF6055
ggcccagccggccatggcccaggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaacccttctagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcacc

SEQ ID NO: 150 - protein, 124 aa, Artificial Sequence
  MF6055
QVQLVQSGADVKKPGASVKVSCKASGYTFTGYYMHWVRQAPGQALEWMGWINPSSGGTNYAKKFQGRVTMTRETSTSTAYMELSRLRSDDTATYYCARDHGSRHFWSYWGFDYWGQGTLVTVSS

SEQ ID NO: 151 - protein, 5 aa, Artificial Sequence
  MF6055 CDR1
GYYMH

SEQ ID NO: 152 - protein, 17 aa, Artificial Sequence
  MF6055 CDR2
WINPSSGGTNYAKKFQG

SEQ ID NO: 153 - protein, 15 aa, Artificial Sequence
  MF6055 CDR3
DHGSRHFWSYWGFDY

SEQ ID NO: 154 - DNA, 382 bp, Artificial Sequence
  MF6056
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaacccttctagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcacc

SEQ ID NO: 155 - protein, 124 aa, Artificial Sequence
  MF6056
QVQLVQSGADVKKPGASVKVTCKASGYTFTGYYMHWVRQAPGQALEWMGWINPSSGGTNYAKKFQGRVSMTRETSTSTAYMQLSRLRSDDTATYYCARDHGSRHFWSYWGFDYWGQGTLVTVSS

SEQ ID NO: 156 - protein, 5 aa, Artificial Sequence
  MF6056 CDR1
GYYMH

SEQ ID NO: 157 - protein, 17 aa, Artificial Sequence
  MF6056 CDR2
WINPSSGGTNYAKKFQG

SEQ ID NO: 158 - protein, 15 aa, Artificial Sequence
  MF6056 CDR3
DHGSRHFWSYWGFDY

SEQ ID NO: 159 - DNA, 382 bp, Artificial Sequence
  MF6057
ggcccagccggccatggcccaggtgcagctggtgcagtctggggctgatgtgaagaagcctggggcctcagtgaaggtcacgtgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatgcagctgagcaggctgagatctgacgacacggctgtgtattactgtgcaagagatcatggttctcgtcatttctggtcttactggggctttgattattggggccaaggtaccctggtcacc

SEQ ID NO: 160 - protein, 124 aa, Artificial Sequence
  MF6057
QVQLVQSGADVKKPGASVKVTCKASGYTFTGYYMHWVRQAPGQGLEWMGWINPQSGGTNYAQKFQGRVTMTRDTSISTAYMQLSRLRSDDTAVYYCARDHGSRHFWSYWGFDYWGQGTLVTVSS

SEQ ID NO: 161 - protein, 5 aa, Artificial Sequence
  MF6057 CDR1
GYYMH

SEQ ID NO: 162 - protein, 17 aa, Artificial Sequence
  MF6057 CDR2
WINPQSGGTNYAQKFQG

SEQ ID NO: 163 - protein, 15 aa, Artificial Sequence
  MF6057 CDR3
DHGSRHFWSYWGFDY

SEQ ID NO: 164 - DNA, 382 bp, Artificial Sequence
  MF6059
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 165 - protein, 124 aa, Artificial Sequence
  MF6059
QVQLVQSGAEVKKPGASVKVSCKASGYTFTGYYMHWVRQAPGQGLEWMGWINPGSGSTNYAQKFQGRVTMTRDTSISTAYMELSRLRSDDTAVYYCARDHGSRHFWSYWGFDYWGQGTLVTVSS

SEQ ID NO: 166 - protein, 5 aa, Artificial Sequence
  MF6059 CDR1
GYYMH

SEQ ID NO: 167 - protein, 17 aa, Artificial Sequence
  MF6059 CDR2
WINPGSGSTNYAQKFQG

SEQ ID NO: 168 - protein, 15 aa, Artificial Sequence
  MF6059 CDR3
DHGSRHFWSYWGFDY

SEQ ID NO: 169 - DNA, 382 bp, Artificial Sequence
  MF6060
ggcccagccggccatggcccaggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaaccctcaaagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcacc

SEQ ID NO: 170 - protein, 124 aa, Artificial Sequence
  MF6060
QVQLVQSGADVKKPGASVKVSCKASGYTFTGYYMHWVRQAPGQALEWMGWINPQSGGTNYAKKFQGRVTMTRETSTSTAYMELSRLRSDDTATYYCARDHGSRHFWSYWGFDYWGQGTLVTVSS

SEQ ID NO: 171 - protein, 5 aa, Artificial Sequence
  MF6060 CDR1
GYYMH

SEQ ID NO: 172 - protein, 17 aa, Artificial Sequence
  MF6060 CDR2
WINPQSGGTNYAKKFQG

SEQ ID NO: 173 - protein, 15 aa, Artificial Sequence
  MF6060 CDR3
DHGSRHFWSYWGFDY

SEQ ID NO: 174 - DNA, 382 bp, Artificial Sequence
  MF6062
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccacaagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 175 - protein, 124 aa, Artificial Sequence
  MF6062
QVQLVQSGAEVKKPGASVKVSCKASGYTFTGYYMHWVRQAPGQGLEWMGWINPGSGSTNYAQKFQGRVTMTRDTSTSTAYMELSRLRSDDTAVYYCARDHGSRHFWSYWGFDYWGQGTLVTVSS

SEQ ID NO: 176 - protein, 5 aa, Artificial Sequence
  MF6062 CDR1
GYYMH

SEQ ID NO: 177 - protein, 17 aa, Artificial Sequence
  MF6062 CDR2
WINPGSGSTNYAQKFQG

SEQ ID NO: 178 - protein, 15 aa, Artificial Sequence
  MF6062 CDR3
DHGSRHFWSYWGFDY

SEQ ID NO: 179 - DNA, 382 bp, Artificial Sequence
  MF6063
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcaaagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 180 - protein, 124 aa, Artificial Sequence
  MF6063
QVQLVQSGAEVKKPGASVKVSCKASGYTFTGYYMHWVRQAPGQGLEWMGWINPQSGGTNYAKKFQGRVTMTRDTSTSTAYMELSRLRSDDTAVYYCARDHGSRHFWSYWGFDYWGQGTLVTVSS

SEQ ID NO: 181 - protein, 5 aa, Artificial Sequence
  MF6063 CDR1
GYYMH

SEQ ID NO: 182 - protein, 17 aa, Artificial Sequence
  MF6063 CDR2
WINPQSGGTNYAKKFQG

SEQ ID NO: 183 - protein, 15 aa, Artificial Sequence
  MF6063 CDR3
DHGSRHFWSYWGFDY

SEQ ID NO: 184 - DNA, 382 bp, Artificial Sequence
  MF6064
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggaaaggggcttgagtggatgggatggatcaaccctcagagtggtggcacaaactatgcacagaagtttcagggcagggtcacgatgaccagggacacgtccacgagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 185 - protein, 124 aa, Artificial Sequence
  MF6064
QVQLVQSGAEVKKPGASVKVSCKASGYTFTGYYMHWVRQAPGKGLEWMGWINPQSGGTNYAQKFQGRVTMTRDTSTSTAYMELSRLRSDDTAVYYCARDHGSRHFWSYWGFDYWGQGTLVTVSS

SEQ ID NO: 186 - protein, 5 aa, Artificial Sequence
  MF6064 CDR1
GYYMH

SEQ ID NO: 187 - protein, 17 aa, Artificial Sequence
  MF6064 CDR2
WINPQSGGTNYAQKFQG

SEQ ID NO: 188 - protein, 15 aa, Artificial Sequence
  MF6064 CDR3
DHGSRHFWSYWGFDY

SEQ ID NO: 189 - DNA, 382 bp, Artificial Sequence
  MF6066
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcacc

SEQ ID NO: 190 - protein, 124 aa, Artificial Sequence
  MF6066
QVQLVQSGAEVKKPGASVKVSCKASGYTFTGYYMHWVRQAPGQGLEWMGWINPQSGSTNYAQKFQGRVTMTRDTSTSTAYMELSSLRSEDTAVYYCARDHGSRHFWSYWGFDYWGQGTLVTVSS

SEQ ID NO: 191 - protein, 5 aa, Artificial Sequence
  MF6066 CDR1
GYYMH

SEQ ID NO: 192 - protein, 17 aa, Artificial Sequence
  MF6066 CDR2
WINPQSGSTNYAQKFQG

SEQ ID NO: 193 - protein, 15 aa, Artificial Sequence
  MF6066 CDR3
DHGSRHFWSYWGFDY

SEQ ID NO: 194 - DNA, 382 bp, Artificial Sequence
  MF6067
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcacc

SEQ ID NO: 195 - protein, 124 aa, Artificial Sequence
  MF6067
QVQLVQSGAEVKKPGASVKVSCKASGYTFTGYYMHWVRQAPGQGLEWMGWINPQSGGTNYAQKFQGRVTMTRDTSTSTVYMELSSLRSDDTAVYYCARDHGSRHFWSYWGFDYWGQGTLVTVSS

SEQ ID NO: 196 - protein, 5 aa, Artificial Sequence
  MF6067 CDR1
GYYMH

SEQ ID NO: 197 - protein, 17 aa, Artificial Sequence
  MF6067 CDR2
WINPQSGGTNYAQKFQG

SEQ ID NO: 198 - protein, 15 aa, Artificial Sequence
  MF6067 CDR3
DHGSRHFWSYWGFDY

SEQ ID NO: 199 - DNA, 382 bp, Artificial Sequence
  MF6068
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 200 - protein, 124 aa, Artificial Sequence
  MF6068
QVQLVQSGAEVKKPGASVKVSCKASGYTFTGYYMHWVRQAPGQGLEWMGWINPQSGGTNYAQKFQGRVTMTRDTSTSTAYMELSRLRSDDTAVYYCARDHGSRHFWSYWGFDYWGQGTLVTVSS

SEQ ID NO: 201 - protein, 5 aa, Artificial Sequence
  MF6068 CDR1
GYYMH

SEQ ID NO: 202 - protein, 17 aa, Artificial Sequence
  MF6068 CDR2
WINPQSGGTNYAQKFQG

SEQ ID NO: 203 - protein, 15 aa, Artificial Sequence
  MF6068 CDR3
DHGSRHFWSYWGFDY

SEQ ID NO: 204 - DNA, 382 bp, Artificial Sequence
  MF6069
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 205 - protein, 124 aa, Artificial Sequence
  MF6069
QVQLVQSGAEVKKPGASVKVSCKASGYTFTGYYMHWVRQAPGQGLEWMGWINPQSGGTNYAQKFQGRVTMTRDTSISTAYMELSRLRSDDTAVYYCARDHGSRHFWSYWGFDYWGQGTLVTVSS

SEQ ID NO: 206 - protein, 5 aa, Artificial Sequence
  MF6069 CDR1
GYYMH

SEQ ID NO: 207 - protein, 17 aa, Artificial Sequence
  MF6069 CDR2
WINPQSGGTNYAQKFQG

SEQ ID NO: 208 - protein, 15 aa, Artificial Sequence
  MF6069 CDR3
DHGSRHFWSYWGFDY

SEQ ID NO: 209 - DNA, 382 bp, Artificial Sequence
  MF6070
ggcccagccggccatggcccaggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaacccttctgggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcacc

SEQ ID NO: 210 - protein, 124 aa, Artificial Sequence
  MF6070
QVQLVQSGAEVKKPGASVKVSCKASGYTFTSYYMHWVRQAPGQGLEWMGWINPSGGSTNYAQKFQGRVTMTRDTSTSTVYMELSRLRSEDTAVYYCARDHGSRHFWSYWGFDYWGQGTLVTVSS

SEQ ID NO: 211 - protein, 5 aa, Artificial Sequence
  MF6070 CDR1
SYYMH

SEQ ID NO: 212 - protein, 17 aa, Artificial Sequence
  MF6070 CDR2
WINPSGGSTNYAQKFQG

SEQ ID NO: 213 - protein, 15 aa, Artificial Sequence
  MF6070 CDR3
DHGSRHFWSYWGFDY

SEQ ID NO: 214 - DNA, 382 bp, Artificial Sequence
  MF6071
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcacc

SEQ ID NO: 215 - protein, 124 aa, Artificial Sequence
  MF6071
QVQLVQSGAEVKKPGASVKVSCKASGYTFTGYYMHWVRQAPGQGLEWMGWINPSSGSTNYAQKFQGRVTMTRDTSTSTAYMELSSLRSEDTAVYYCARDHGSRHFWSYWGFDYWGQGTLVTVSS

SEQ ID NO: 216 - protein, 5 aa, Artificial Sequence
  MF6071 CDR1
GYYMH

SEQ ID NO: 217 - protein, 17 aa, Artificial Sequence
  MF6071 CDR2
WINPSSGSTNYAQKFQG

SEQ ID NO: 218 - protein, 15 aa, Artificial Sequence
  MF6071 CDR3
DHGSRHFWSYWGFDY

SEQ ID NO: 219 - DNA, 382 bp, Artificial Sequence
  MF6072
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcacc

SEQ ID NO: 220 - protein, 124 aa, Artificial Sequence
  MF6072
QVQLVQSGAEVKKPGASVKVSCKASGYTFTGYYMHWVRQAPGQGLEWMGWINPSSGGTNYAQKFQGRVTMTRDTSTSTVYMELSSLRSDDTAVYYCARDHGSRHFWSYWGFDYWGQGTLVTVSS

SEQ ID NO: 221 - protein, 5 aa, Artificial Sequence
  MF6072 CDR1
GYYMH

SEQ ID NO: 222 - protein, 17 aa, Artificial Sequence
  MF6072 CDR2
WINPSSGGTNYAQKFQG

SEQ ID NO: 223 - protein, 15 aa, Artificial Sequence
  MF6072 CDR3
DHGSRHFWSYWGFDY

SEQ ID NO: 224 - DNA, 382 bp, Artificial Sequence
  MF6073
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 225 - protein, 124 aa, Artificial Sequence
  MF6073
QVQLVQSGAEVKKPGASVKVSCKASGYTFTGYYMHWVRQAPGQGLEWMGWINPSSGGTNYAQKFQGRVTMTRDTSTSTAYMELSRLRSDDTAVYYCARDHGSRHFWSYWGFDYWGQGTLVTVSS

SEQ ID NO: 226 - protein, 5 aa, Artificial Sequence
  MF6073 CDR1
GYYMH

SEQ ID NO: 227 - protein, 17 aa, Artificial Sequence
  MF6073 CDR2
WINPSSGGTNYAQKFQG

SEQ ID NO: 228 - protein, 15 aa, Artificial Sequence
  MF6073 CDR3
DHGSRHFWSYWGFDY

SEQ ID NO: 229 - DNA, 382 bp, Artificial Sequence
  MF6074
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcacc

SEQ ID NO: 230 - protein, 124 aa, Artificial Sequence
  MF6074
QVQLVQSGAEVKKPGASVKVSCKASGYTFTGYYMHWVRQAPGQGLEWMGWINPSSGGTNYAQKFQGRVTMTRDTSISTAYMELSRLRSDDTAVYYCARDHGSRHFWSYWGFDYWGQGTLVTVSS

SEQ ID NO: 231 - protein, 5 aa, Artificial Sequence
  MF6074 CDR1
GYYMH

SEQ ID NO: 232 - protein, 17 aa, Artificial Sequence
  MF6074 CDR2
WINPSSGGTNYAQKFQG

SEQ ID NO: 233 - protein, 15 aa, Artificial Sequence
  MF6074 CDR3
DHGSRHFWSYWGFDY